(19) United States
(12) Patent Application Publication
(10) Pub. No.: US 2026/0117288 A1
(43) Pub. Date: Apr. 30, 2026

(54) METHODS FOR SPECIFIC DETECTION OF NUCLEIC ACID SEQUENCES USING IN VITRO TRANSCRIPTION AND IN SITU SEQUENCING

(71) Applicant: Waypoint Bio, Inc., New York, NY (US)

(72) Inventors: Cassandra Nicole Kontur, Brooklyn, NY (US); David Vincent Phizicky, Jersey City, NJ (US); Xinchen Wang, New York, NY (US); Megha Sah, New York, NY (US)

(21) Appl. No.: 19/094,458

(22) Filed: Mar. 28, 2025

Related U.S. Application Data
(63) Continuation of application No. 18/975,326, filed on Dec. 10, 2024.
Application No. 18/975,326 is a continuation of application No. PCT/US2024/034104, filed on Jun. 14, 2024.
(60) Provisional application No. 63/508,353, filed on Jun. 15, 2023.

(51) Int. Cl. C12Q 1/6841; C12Q 1/682; C12Q 1/6869

(52) U.S. Cl. CPC C12Q 1/6841; C12Q 1/6869; C12Q 1/682

(57) ABSTRACT

The present disclosure relates, in general, to methods and compositions for detecting one or more nucleic acid sequences of interest (e.g., nucleic acid barcodes) in a biological sample in situ.

### CROSS-REFERENCE TO RELATED APPLICATIONS

This application is a continuation of U.S. patent application Ser. No. 18/975,326, filed on Dec. 10, 2024, which is a continuation of International (PCT) Application No. PCT/US2024/034104, filed on Jun. 14, 2024, which claims the benefit of and priority to U.S. Provisional Patent Application No. 63/508,353, filed on Jun. 15, 2023, the disclosures of each of which are hereby incorporated by reference in their entireties for all purposes.

### BACKGROUND

Fluorescence microscopy is one of the most important, pervasive and powerful imaging modalities in biomedical research. The spatial resolution of modern fluorescence microscopy has been improved to such a point that even sub-diffraction limited resolution is routinely possible. It is often desirable to visualize specific nucleic acid sequences in situ for various spatial biology applications. However, current methods require numerous steps that combine to yield poor sensitivity for low-abundance nucleic acid species, particularly in cells that have inherently low mRNA production. Such limitations are particularly evident for methods such as fluorescence in situ sequencing (FISSEQ), which are hampered by low sensitivity. It is therefore desirable to produce new methods with higher sensitivity, particularly for low-abundance nucleic acid species.

### SUMMARY

The present disclosure relates, in general, to methods and compositions for detecting one or more nucleic acid sequences of interest in a biological sample in situ.

Disclosed herein, in various embodiments, is a method of determining the presence, absence, amount, and/or localization of a nucleic acid sequence of interest in one or more fixed mammalian cells within a biological sample in situ, the method comprising: (a) reacting, within the one or more fixed mammalian cells, (i) a DNA molecule comprising the nucleic acid sequence of interest operably linked to a sequence-specific RNA polymerase promoter, and (ii) a sequence-specific RNA polymerase to generate an RNA transcript of the nucleic acid sequence of interest; (b) reacting the RNA transcript in situ with a reverse transcriptase enzyme to generate a cDNA molecule comprising the nucleic acid sequence of interest; and (c) in situ sequencing the cDNA molecule to visualize the nucleic acid sequence of interest in the one or more fixed mammalian cells. In some embodiments, the method comprises, prior to step (a), contacting the fixed mammalian cell with an RNase to degrade endogenous RNA molecules.

In some embodiments, the DNA molecule is or is derived from an exogenous nucleic acid molecule that is introduced to the one or more mammalian cells prior to fixation. In some embodiments, the nucleic acid sequence of interest is a barcode polynucleotide. In some embodiments, the exogenous nucleic acid molecule is incorporated into a genome of the one or more mammalian cells by viral transduction, site-specific nucleases, or site-specific recombinases. In some embodiments, the exogenous DNA molecule is introduced to the one or more mammalian cells using a viral vector selected from a lentiviral vector, a retroviral vector, an adenovirus vector, an HSV vector, a baculovirus vector, a virus-like particle, a pseudotyped virus-like capsid, an oncolytic viral vector, or an AAV vector. In some embodiments, the exogenous nucleic acid sequence is incorporated at a specific site in the genome. In some embodiments, the exogenous nucleic acid sequence is incorporated at a random site in the genome. In some embodiments, the exogenous nucleic acid is not integrated into a mammalian chromosome. In some embodiments, the exogenous nucleic acid molecule is retained in a nucleus of the one or more mammalian cells. In some embodiments, the exogenous nucleic acid molecule is comprised within a plasmid or an artificial chromosome.

In some embodiments, the nucleic acid sequence of interest is an endogenous nucleic acid sequence and the promoter is an exogenous promoter. In some embodiments, the endogenous nucleic acid sequence is variable between cells within the biological sample. In some embodiments, the endogenous nucleic acid sequence encodes a T cell receptor, a B cell receptor, an immunoglobulin sequence, a repeat sequence, or a region comprising a somatic mutation. In some embodiments, the nucleic acid sequence of interest is an endogenous sequence that does not vary between cells within the biological sample.

In some embodiments, the DNA molecule is generated by reverse transcribing with a DNA primer that hybridizes to a target RNA comprising the nucleic acid sequence of interest in the one or more fixed mammalian cells, wherein the DNA primer comprises: (i) a 5′ nucleic acid sequence comprising a sequence-specific RNA polymerase promoter, and (ii) a 3′ nucleic acid sequence that is complementary to a portion of the target RNA flanking the nucleic acid sequence of interest. In some embodiments, the method further comprises converting the DNA molecule to double-stranded DNA by second-strand synthesis. In some embodiments, the 5′ nucleic acid sequence of the DNA primer comprising the sequence-specific RNA polymerase promoter is dsDNA. In some embodiments, the dsDNA is hybridized dsDNA or a hairpin. In some embodiments, the target RNA molecule is wholly or partially digested following synthesis of the DNA molecule. In some embodiments, the in situ sequencing is sequencing-by-synthesis, sequencing-by-ligation, or sequencing-by-avidity. In some embodiments, the in situ sequencing is sequencing-by-synthesis.

In some embodiments, the sequence-specific RNA polymerase promoter is a phage promoter, or a transcriptionally active variant thereof, and the sequence-specific RNA polymerase is a phage RNA polymerase. In some embodiments, the sequence-specific RNA polymerase promoter and the sequence-specific RNA polymerase are selected from the group consisting of: (i) a T7 promoter, or a transcriptionally active variant thereof, and a T7 RNA polymerase, respectively; (ii) a T3 promoter, or a transcriptionally active variant thereof, and a T3 RNA polymerase, respectively; and (iii) an SP6 promoter, or a transcriptionally active variant thereof, and an SP6 RNA polymerase, respectively. In some embodiments, the promoter is a T7 promoter and the RNA polymerase is a T7 RNA polymerase.

In some embodiments, the sequence-specific RNA polymerase promoter is a bacterial promoter and the sequence-specific RNA polymerase is a bacterial RNA polymerase. In some embodiments, the sequence-specific RNA polymerase promoter is a eukaryotic promoter and the sequence-specific RNA polymerase is a eukaryotic RNA polymerase. In some embodiments, the sequence-specific RNA polymerase promoter is a viral promoter and the sequence-specific RNA polymerase is a viral RNA polymerase. In some embodiments, the sequence-specific RNA polymerase promoter is a synthetic promoter and the sequence-specific RNA polymerase is a synthetic RNA polymerase.

In some embodiments, the DNA molecule further comprises a transcriptional terminator. In some embodiments, the transcriptional terminator is a T7 terminator. In some embodiments, the biological sample is fixed using a solution comprising formaldehyde and/or paraformaldehyde. In some embodiments, the solution comprises 4% paraformaldehyde. In some embodiments, the biological sample comprises a formalin-fixed, paraffin-embedded (FFPE) sample comprising the one or more mammalian cells. In some embodiments, the biological sample is fixed by cryofixation. In some embodiments, the sample comprises optimal cutting temperature compound, a hydrogel matrix, or a swellable polymer hydrogel. In some embodiments, the sample is fixed using a solution comprising an alcohol. In some embodiments, the alcohol is methanol or ethanol. In some embodiments, the sample is fixed using a solution comprising glutaraldehyde.

In some embodiments, the nucleic acid sequence of interest is less than 100, less than 90, less than 80, less than 70, less than 60, less than 50, less than 40, less than 30, less than 25, less than 20, less than 15, less than 10, or less than 5 nucleotides in length. In some embodiments, the DNA molecule further comprises one or more polynucleotide sequences encoding exogenous proteins, endogenous proteins, or a mixture of exogenous and endogenous proteins. In some embodiments, the DNA molecule further comprises a polynucleotide sequence encoding one or more exogenous proteins. In some embodiments, at least a subset of the one or more exogenous proteins are synthetic proteins and/or chimeric proteins. In some embodiments, the one or more exogenous proteins are independently selected from the group consisting of a chimeric antigen receptor (CAR), an antibody, a T-cell receptor, a cytokine, a cell-surface receptor, a transcription factor, a signaling protein, and a protease. In some embodiments, two or more exogenous proteins are expressed. In some embodiments, expression of the exogenous protein is controlled by proteins endogenous to the one or more mammalian cells. In some embodiments, the DNA molecule further comprises a polynucleotide sequence encoding an endogenous protein. In some embodiments, the DNA molecule further comprises a polynucleotide sequence encoding an endogenous RNA. In some embodiments, the DNA molecule further comprises a polynucleotide sequence encoding an exogenous RNA. In some embodiments, the DNA molecule further comprises a polynucleotide sequence encoding a nucleic acid sequence that alters expression, function, and/or sequence of one or more genes. In some embodiments, the nucleic acid sequence that alters expression, function, and/or sequence of one or more genes is selected from the group consisting of an sgRNA, a gRNA, an shRNA, and an miRNA. In some embodiments, the DNA molecule further comprises a polynucleotide sequence encoding a viral genome. In some embodiments, the viral genome is an oncolytic viral genome.

In some embodiments, the DNA molecule comprises a second sequence-specific RNA polymerase promoter configured to drive transcription of a second nucleic acid sequence of interest in the presence of a second sequence-specific RNA polymerase. In some embodiments, the second nucleic acid sequence of interest is a second barcode polynucleotide. In some embodiments, the second sequence-specific RNA polymerase promoter and the second sequence-specific RNA polymerase are selected from the group consisting of: (i) a T7 promoter, or a transcriptionally active variant thereof, and a T7 RNA polymerase, respectively; (ii) a T3 promoter, or a transcriptionally active variant thereof, and a T3 RNA polymerase, respectively; and (iii) a SP6 promoter, or a transcriptionally active variant thereof, and a SP6 RNA polymerase, respectively. In some embodiments, the second sequence-specific RNA polymerase promoter is a T7 promoter, or a transcriptionally active variant thereof, and the second sequence-specific RNA polymerase is a T7 RNA polymerase. In some embodiments, the second sequence-specific RNA polymerase promoter is a bacterial promoter or a transcriptionally active variant thereof and the second sequence-specific RNA polymerase is a bacterial RNA polymerase.

In some embodiments the second sequence-specific RNA polymerase promoter is a eukaryotic promoter or a transcriptionally active variant thereof and the second sequence-specific RNA polymerase is a eukaryotic RNA polymerase. In some embodiments, the second sequence-specific RNA polymerase promoter is a viral promoter or a transcriptionally active variant thereof and the second sequence-specific RNA polymerase is a viral RNA polymerase. In some embodiments, the second sequence-specific RNA polymerase promoter is a synthetic promoter and the second sequence-specific RNA polymerase is a synthetic RNA polymerase. In some embodiments, the first and second promoters and RNA polymerases are the same. In some embodiments, the first and second promoters and RNA polymerases are different. In some embodiments, the nucleic acid sequence of interest and the second nucleic acid sequence of interest flank the polynucleotide encoding the exogenous protein. In some embodiments, the nucleic acid sequence of interest and the second nucleic acid sequence of interest were introduced on the same nucleic acid. In some embodiments, the nucleic acid sequence of interest and the nucleic acid sequence of interest were introduced on different nucleic acids. In some embodiments, the DNA molecule comprises three or more, four or more, five or more, six or more, seven or more, eight or more, nine or more, or ten or more sequence-specific RNA polymerase promoters configured to each drive transcription of a distinct nucleic acid sequence of interest in the presence of a distinct sequence-specific RNA polymerase.

In some embodiments, the DNA molecule further comprises a first padlock-binding sequence and a second padlock-binding sequence, wherein said first and second padlock-binding sequences flank a region comprising the nucleic acid sequence of interest. In some embodiments, the DNA molecule further comprises a third padlock-binding sequence and a fourth padlock-binding sequence, wherein said third and fourth padlock-binding sequences flank a region comprising the second nucleic acid sequence of interest. In some embodiments, prior to in situ sequencing, step (c) further comprises the steps of: (i) contacting the cDNA with a first padlock probe comprising a 5′ end and a 3′ end, wherein the first padlock probe comprises a 5′ nucleic acid sequence which is reverse complementary to the first padlock-binding site and a 3′ nucleic acid sequence which is reverse complementary to the second padlock-binding site, thereby allowing the 5′ and 3′ nucleic acid sequences to hybridize to the cDNA; (ii) extending the 3′ end of the first padlock probe through the nucleic acid sequence of interest using a DNA polymerase; (iii) ligating the 5′ end of the padlock probe to the extended 3′ end of the padlock probe, thereby generating a circular DNA template comprising a nucleic acid sequence reverse complementary to the nucleic acid sequence of interest; and (iv) using rolling circle amplification of the DNA template to generate additional copies of the nucleic acid sequence of interest. In some embodiments, step (i) further comprises contacting the cDNA with a second padlock probe comprising a 5′ end and a 3′ end, wherein the second padlock probe comprises a 5′ nucleic acid sequence which is reverse complementary to the third padlock-binding site and a 3′ nucleic acid sequence which is reverse complementary to the fourth padlock-binding site; and step (ii) further comprises extending the 3′ end of the second padlock probe through the second nucleic acid sequence of interest using a DNA polymerase. In some embodiments, the first and second padlock-binding sequences are different from the third and fourth padlock-binding sequences. In some embodiments, the first and second padlock-binding sequences are identical to the third and fourth padlock-binding sequences. In some embodiments, the in situ sequencing is performed directly on the cDNA.

In some embodiments, the biological sample comprises one or more immune cells. In some embodiments, the one or more immune cells are T cells, NK cells, B cells, mast cells, dendritic cells, macrophages, neutrophils, basophils, and/or eosinophils. In some embodiments, the biological sample comprises a mixture of cells from different species. In some embodiments, the biological sample comprises human cells and mouse cells. In some embodiments, the biological sample comprises human immune cells and mouse cells. In some embodiments, the one or more cells within the biological sample consist of cells from a single species. In some embodiments, the one or more cells within the biological sample consist of human cells. In some embodiments, the biological sample comprises one or both of cancer cells and fibroblast cells. In some embodiments, the biological sample comprises one or more human cancer cells. In some embodiments, the biological sample comprises one or more murine cancer cells. In some embodiments, the biological sample comprises one or more nervous system cells. In some embodiments, the nervous system cells comprise one or more of neurons, astrocytes, and microglia.

In some embodiments, less than 100% of the cells in the biological sample comprise the nucleic acid sequence of interest. In some embodiments, the biological sample comprises an FFPE sample and less than 100% of the cells in the biological sample comprise the nucleic acid sequence of interest. In some embodiments, all or substantially all of the cells in the biological sample comprise the nucleic acid sequence of interest.

Also disclosed herein, in various embodiments, is a method of determining the presence, absence, amount, and/or localization of a nucleic acid sequence of interest in one or more fixed mammalian cells within a biological sample in situ, the method comprising: (a) reverse transcribing with a DNA primer a target RNA comprising the nucleic acid sequence of interest in the one or more fixed mammalian cells to generate a first cDNA molecule comprising the nucleic acid sequence of interest, wherein the DNA primer comprises: (i) a 5′ nucleic acid sequence comprising a sequence-specific RNA polymerase promoter; and (ii) a 3′ nucleic acid sequence that is complementary to a portion of the target RNA flanking the nucleic acid sequence of interest, wherein the DNA primer hybridizes to the target RNA; wherein the first cDNA molecule comprises the sequence-specific RNA polymerase promoter operably linked to the nucleic acid sequence of interest; (b) reacting the first cDNA molecule with a sequence-specific RNA polymerase to generate an RNA transcript comprising the nucleic acid sequence of interest; (c) reacting the RNA transcript with a reverse transcriptase enzyme to generate a second cDNA molecule comprising the nucleic acid sequence of interest; and (d) in situ sequencing the second cDNA molecule to visualize the nucleic acid sequence of interest in the one or more fixed mammalian cells.

In some embodiments, the method further comprises, prior to step (b), using second strand synthesis to convert the first cDNA molecule to double-stranded DNA. In some embodiments, the 5′ nucleic acid sequence of the DNA primer comprising the sequence-specific RNA polymerase promoter is dsDNA. In some embodiments, the dsDNA is hybridized dsDNA or a hairpin. In some embodiments, the method comprises, prior to step (b), contacting the fixed mammalian cell with an RNase to degrade endogenous RNA molecules.

In some embodiments, the nucleic acid sequence of interest is or is derived from an exogenous nucleic acid sequence that is introduced to the one or more mammalian cells prior to fixation. In some embodiments, the nucleic acid sequence of interest is a barcode polynucleotide. In some embodiments, the exogenous nucleic acid sequence is incorporated into a genome of the one or more mammalian cells by viral transduction, site-specific nucleases, or site-specific recombinases. In some embodiments, the exogenous nucleic acid sequence is introduced to the mammalian cell using a viral vector selected from a lentiviral vector, a retroviral vector, an adenovirus vector, an HSV vector, a baculovirus vector, a virus-like particle, a pseudotyped virus-like capsid, or an AAV vector. In some embodiments, the exogenous nucleic acid sequence is incorporated at a pre-selected locus in the genome. In some embodiments, the exogenous nucleic acid sequence is incorporated at a random locus in the genome. In some embodiments, the exogenous nucleic acid is not integrated into a mammalian chromosome. In some embodiments, the exogenous nucleic acid is retained in a nucleus of the one or more cells. In some embodiments, the exogenous nucleic acid is comprised within a plasmid or an artificial chromosome.

In some embodiments, the nucleic acid sequence of interest is an endogenous sequence that is variable between cells within the biological sample. In some embodiments, the endogenous nucleic acid sequence encodes a T cell receptor, a B cell receptor, an immunoglobulin sequence, a repeat sequence, or a region containing a somatic mutation. In some embodiments, the nucleic acid sequence of interest is an endogenous sequence that does not vary between cells within the biological sample.

In some embodiments, the RNA molecule is an mRNA. In some embodiments, the RNA molecule is a non-coding RNA. In some embodiments, the RNA molecule comprises a gRNA. In some embodiments, the in situ sequencing is sequencing-by-synthesis, sequencing-by-ligation, or sequencing-by-avidity. In some embodiments, the in situ sequencing is sequencing-by-synthesis. In some embodiments, the nucleic acid sequence of interest is less 100, less than 90, less than 80, less than 70, less than 60, less than 50, less than 40, less than 30, less than 25, less than 20, less than 15, less than 10, or less than 5 nucleotides in length.

In some embodiments, the promoter is a phage promoter or a transcriptionally active variant thereof and the sequence-specific RNA polymerase is a phage RNA polymerase. In some embodiments, the promoter and the sequence-specific RNA polymerase are selected from the group consisting of: (i) a T7 promoter, or a transcriptionally active variant thereof, and a T7 RNA polymerase, respectively; (ii) a T3 promoter, or a transcriptionally active variant thereof, and a T3 RNA polymerase, respectively; and (iii) an SP6 promoter, or a transcriptionally active variant thereof, and an SP6 RNA polymerase, respectively. In some embodiments, the promoter is a T7 promoter, or a transcriptionally active variant thereof, and the RNA polymerase is a T7 RNA polymerase.

In some embodiments, the sequence-specific RNA polymerase promoter is a bacterial promoter, or a transcriptionally active variant thereof, and the sequence-specific RNA polymerase is a bacterial RNA polymerase. In some embodiments, the sequence-specific RNA polymerase promoter is a eukaryotic promoter, or a transcriptionally active variant thereof, and the sequence-specific RNA polymerase is a eukaryotic RNA polymerase. In some embodiments, the sequence-specific RNA polymerase promoter is a viral promoter, or a transcriptionally active variant thereof, and the sequence-specific RNA polymerase is a viral RNA polymerase. In some embodiments, the sequence-specific RNA polymerase promoter is a synthetic promoter and the sequence-specific RNA polymerase is a synthetic RNA polymerase.

In some embodiments, the biological sample is fixed using a solution comprising formaldehyde and/or paraformaldehyde. In some embodiments, the solution comprises 4% paraformaldehyde. In some embodiments, the biological sample comprises a formalin-fixed, paraffin-embedded (FFPE) sample comprising the one or more mammalian cells. In some embodiments, the biological sample is fixed by cryofixation. In some embodiments, the sample comprises, optimal cutting temperature compound, a hydrogel matrix, or a swellable polymer hydrogel. In some embodiments, the sample is fixed using a solution comprising an alcohol. In some embodiments, the alcohol is methanol or ethanol. In some embodiments, the sample is fixed using a solution comprising glutaraldehyde.

In some embodiments, the target RNA encodes an exogenous protein, an endogenous protein, or a mixture of exogenous and endogenous proteins. In some embodiments, the target RNA encodes an exogenous protein. In some embodiments, the exogenous proteins is a synthetic protein and/or a chimeric protein. In some embodiments, the exogenous protein is selected from the group consisting of a chimeric antigen receptor (CAR), an antibody, a T-cell receptor, a cytokine, a cell-surface receptor, a transcription factor, a signaling protein, and a protease. In some embodiments, expression of the exogenous protein is controlled by proteins endogenous to the one or more mammalian cells. In some embodiments, the target RNA encodes a nucleic acid sequence that alters expression, function, and/or sequence of one or more genes is selected from the group consisting of an sgRNA, a gRNA, an shRNA, and an miRNA. In some embodiments, the target RNA further comprises a first padlock-binding sequence and a second padlock-binding sequence, wherein said first and second padlock-binding sequences flank the nucleic acid sequence of interest. In some embodiments, prior to carrying out in situ sequencing, step (d) further comprises the steps of: (i) contacting the second cDNA molecule with a padlock probe comprising a 5′ end and a 3′ end, wherein the padlock probe comprises a 5′ nucleic acid sequence that is reverse complementary to the first padlock-binding site and a 3′ nucleic acid sequence that is reverse complementary to the second padlock-binding site, thereby allowing the 5′ and 3′ nucleic acid sequences of the padlock probe to hybridize to the second cDNA molecule; (ii) extending the 3′ end of the padlock probe using a DNA polymerase; (iii) ligating the 5′ end of the padlock probe to the extended 3′ end of the padlock probe, thereby generating a circular DNA template comprising a nucleic acid sequence reverse complementary to the nucleic acid sequence of interest; and (iv) using rolling circle amplification of the DNA template to generate additional copies of the nucleic acid sequence of interest.

In some embodiments, the biological sample comprises one or more immune cells. In some embodiments, the one or more immune cells are T cells, NK cells, B cells, mast cells, dendritic cells, macrophages, neutrophils, basophils, and/or eosinophils. In some embodiments, the biological sample comprises a mixture of cells from different species. In some embodiments, the biological sample comprises human cells and mouse cells. In some embodiments, the biological sample comprises human immune cells and mouse cells. In some embodiments, the one or more cells within the biological sample consist of cells from a single species. In some embodiments, the one or more cells within the biological sample consist of human cells.

In some embodiments, less than 100% of the cells in the biological sample comprise the nucleic acid sequence of interest. In some embodiments, the biological sample comprises an FFPE sample and wherein less than 100% of the cells in the biological sample comprise the nucleic acid sequence of interest. In some embodiments, all or substantially all of the cells in the biological sample comprise the nucleic acid sequence of interest.

In some embodiments, the method further comprises detecting the presence, absence, amount and/or localization of one or more additional analytes in the cells. In some embodiments, the one or more additional analytes are independently selected from the group consisting of protein, RNA, DNA stained in a non-sequence specific manner, DNA with a specific sequence, DNA mutations, lipids, including but not limited to phospholipids and sphingolipids, carbohydrates including but not limited to monosaccharides and polysaccharides, metabolites, small molecules, cellular structures, and tissue structures. In some embodiments, the method further comprises detecting the presence, absence, amount and/or localization of one or more protein analytes in the cells. In some embodiments, the one or more protein analytes are detected by immunofluorescence microscopy. In some embodiments, the method further comprises detecting the presence, absence, amount and/or localization of one or more RNA analytes in the cells. In some embodiments, the one or more RNA analytes are detected by hybridization chain reaction (HCR).

Also disclosed herein, in various embodiments, is a kit comprising: (a) a sequence-specific RNA polymerase; (b) a reverse transcriptase enzyme; (c) reagents for in situ sequencing; and (d) instructions for using components (a)-(c) to determine the presence, absence, amount, and/or localization of a nucleic acid sequence of interest in one or more fixed mammalian cells in a biological sample in situ. In some embodiments, (i) the in situ sequencing is sequencing-by-synthesis and the reagents for in situ sequencing comprise (A) a plurality of detectably labeled nucleotides and (B) a DNA polymerase; (ii) the in situ sequencing is sequencing-by-ligation and the reagents for in situ sequencing comprise (A) a plurality of detectably labeled oligonucleotides comprising degenerate bases and (B) a DNA ligase; or (iii) the in situ sequencing is sequencing-by-avidity and the reagents for in situ sequencing comprise (A) a plurality of detectably labeled avidites and (B) an engineered DNA polymerase.

In some embodiments, the kit further comprises: (i) a DNA primer comprising: (A) a 3′ nucleic acid sequence that is complementary to a portion of a target RNA flanking the nucleic acid sequence of interest, and (B) a 5′ nucleic acid sequence comprising a sequence-specific RNA polymerase promoter; or further instructions for designing said DNA primer; and (ii) further instructions for performing reverse transcription on the target RNA using the DNA primer to generate a cDNA molecule. In some embodiments, the 5′ nucleic acid sequence of the DNA primer comprising the sequence-specific RNA polymerase promoter is dsDNA. In some embodiments, the dsDNA is hybridized dsDNA or a hairpin. In some embodiments, the kit further comprises: (i) reagents for performing second strand synthesis on the cDNA molecule; and/or (ii) further instructions for performing said second strand synthesis.

Also disclosed herein, in various embodiments, is a population of engineered cells comprising an exogenous promoter juxtaposed to an endogenous genomic region comprising a genomic sequence that is variable between cells within the population, wherein the exogenous promoter is capable of driving expression of the genomic sequences that are variable between cells within the population, and wherein the exogenous promoter was inserted in a site-specific manner. In some embodiments, the exogenous promoter is selected from the group consisting of a T7 promoter, a T3 promoter, and an SP6 promoter. In some embodiments, the promoter is a T7 promoter. In some embodiments, the exogenous promoter and the genomic sequence that is variable between cells within the population are separated by no more than about 2 kilobases (kb), no more than 1.5 kb, no more than 1 kb, no more than 900 (base pairs) bp, no more than 800 bp, no more than 700 bp, no more than 600 bp, no more than 500 bp, no more than 400 bp, no more than 300 bp, no more than 250 bp, no more than 200 bp, no more than 150 bp, no more than 100 bp, no more than 50 bp, or 0 bp. In some embodiments, the separation is in a genomic DNA sequence, an exonic coding sequence, or an RNA sequence of the cell

In some embodiments, the engineered cells are mammalian cells. In some embodiments, the mammalian cells are human cells. In some embodiments, the engineered cells are immune cells. In some embodiments, the immune cells are T cells, NK cells, B cells, mast cells, dendritic cells, macrophages, neutrophils, basophils, and/or eosinophils. In some embodiments, the genomic sequences that are variable between cells within the population encode a T cell receptor, a B cell receptor, an immunoglobulin sequence, a repeat sequence, or a region comprising a somatic mutation.

Also disclosed herein, in various embodiments, is a method of determining the presence, absence, amount, and/or localization of a nucleic acid sequence of interest in one or more mammalian cells within a biological sample in situ, the method comprising: (a) introducing into the one or more mammalian cells an exogenous DNA molecule comprising the nucleic acid sequence of interest operably linked to a sequence specific RNA polymerase promoter; (b) fixing the mammalian cells; (c) reacting the exogenous DNA molecule with a sequence-specific RNA polymerase to generate an RNA transcript of the nucleic acid sequence of interest; (d) reacting the RNA transcript in situ with a reverse transcriptase enzyme to generate a cDNA molecule comprising the nucleic acid sequence of interest; and (c) in situ sequencing the cDNA molecule to visualize a copy of the nucleic acid sequence of interest in the one or more fixed mammalian cells. In some embodiments, the exogenous DNA molecule is genetically engineered into the genome of the one or more mammalian cells. In some embodiments, the exogenous DNA molecule is genetically engineered upstream to one or more genetic loci of interest.

Also disclosed herein, in various embodiments, is a method of determining the presence, absence, amount, and/or localization of a nucleic acid sequence of interest in one or more mammalian cells within a biological sample in situ, the method comprising: (a) fixing the mammalian cells; (b) introducing into the one or more mammalian cells a DNA primer comprising: (i) a 5′ nucleic acid sequence comprising a sequence-specific RNA polymerase promoter; and (ii) a 3′ nucleic acid sequence that is complementary to a portion of the target RNA flanking the nucleic acid sequence of interest; wherein the DNA primer hybridizes to a target RNA comprising the nucleic acid sequence of interest; (c) reverse transcribing the target RNA with the DNA primer to generate a first cDNA molecule comprising the nucleic acid sequence of interest operably linked to the nucleic acid sequence of interest; (d) reacting the first cDNA molecule with a sequence-specific RNA polymerase to generate an RNA transcript comprising the nucleic acid sequence of interest; (c) reacting the RNA transcript with a reverse transcriptase enzyme to generate a second cDNA molecule comprising the nucleic acid sequence of interest; and (f) in situ sequencing the second cDNA molecule to visualize the nucleic acid sequence of interest in the one or more fixed mammalian cells.

These and other aspects and features of the invention are described in the following detailed description and claims.

### BRIEF DESCRIPTION OF THE DRAWINGS

The invention can be more completely understood with reference to the following drawings. It is noted that wherever practicable similar or like reference numbers may be used in the figures and may indicate similar or like functionality

FIGS. 1A-1B depict a schematic of an exemplary method provided herein, according to an embodiment. FIG. 1A depicts a schematic of an exemplary method in which, within a fixed cell, a T7 promoter and a T7 RNA polymerase are used to drive transcription of a nucleic acid barcode, which is then visualized with subsequent reverse transcription and sequencing-by-synthesis. FIG. 1B depicts a schematic in which, within a fixed cell, a nucleic acid sequence of interest is amplified using a sequence-specific RNA polymerase and subsequently visualized by reverse transcription and sequencing-by-synthesis.

FIGS. 2A-2C depicts a schematic of an exemplary method provided herein, according to an embodiment. FIG. 2A depicts a schematic of an exemplary method in which, within a fixed cell, an exogenous T7 promoter is introduced to a nucleic acid barcode, which is then amplified using T7 RNA polymerase and subsequently visualized with subsequent second-strand synthesis, reverse transcription, and sequencing-by-synthesis. FIG. 2B depicts a schematic of an exemplary method in which, within a fixed cell, an exogenous T7 promoter is introduced to a nucleic acid barcode using a primer comprising a double-stranded portion comprising the T7 promoter. The nucleic acid barcode is then amplified using T7 RNA polymerase and subsequently visualized with subsequent reverse transcription and sequencing-by-synthesis. FIG. 2C depicts the synthesis of a cDNA molecule comprising an exogenous promoter operably linked to a nucleic acid sequence of interest by introduction of a primer comprising the exogenous promoter and a nucleic acid sequence that partially overlaps with the nucleic acid sequence of interest.

FIG. 3 depicts microscopy images of cultured cells that have undergone reverse transcription and in situ sequencing-by-synthesis with (bottom panels) or without (top panels) an initial step of barcode amplification with T7 RNA polymerase.

FIG. 4 depicts microscopy images of formalin-fixed paraffin-embedded (FFPE) tissue sections that have undergone reverse transcription and in situ sequencing-by-synthesis with (bottom panels) or without (top panels) an initial step of barcode amplification with T7 RNA polymerase.

FIG. 5 depicts microscopy images of formalin-fixed paraffin-embedded (FFPE) tissue sections that have undergone reverse transcription and in situ sequencing-by-synthesis with an initial step of barcode amplification with T7 polymerase of 3 hours (top panels) or 18 hours (bottom panels).

FIG. 6 depicts microscopy images of formalin-fixed paraffin-embedded (FFPE) tissue sections that have undergone barcode amplification with T7 RNA polymerase and subsequent reverse transcription and in situ sequencing-by-synthesis. Panels depict tissues with (bottom panels) or without (top panels) an initial RNase treatment to degrade native mRNA prior to reverse transcription.

FIG. 7 depicts microscopy images of formalin-fixed paraffin-embedded (FFPE) tissue sections that have undergone barcode amplification with T7 RNA polymerase and subsequent reverse transcription and in situ sequencing-by-synthesis. Panels depict tissue that has undergone indicated number of rounds of sequencing-by-synthesis.

FIG. 8 depicts a schematic of an exemplary workflow for amplification of variable barcode sequence using an integrated promoter within a biological sample, followed by amplification of the cDNA product.

FIGS. 9A-9C depict microscopy images of an FFPE tissue section from an orthotopic gastric patient-derived xenograft tumor mouse model with 7 unique CAR designs following barcode amplification with a T7 RNA polymerase, reverse transcription, cDNA amplification, and in situ sequencing-by-synthesis (SBS). FIG. 9A depicts DAPI signal from the whole tumor with a 1 mm scale bar. FIG. 9B depicts tissue comprising the boxed region in FIG. 9A with DAPI signal overlayed with SBS signal from all nucleotides (A,G,C,T) in round 1 with a 100 μm scale bar. FIG. 9C depicts tissue comprising the boxed region in FIG. 9B with signal from each fluorescent channel in each round of SBS to depict the barcode readouts over multiple rounds. Dashed-line shapes indicate three individual cells in this field-of-view with distinct amplified barcodes, each corresponding to one of the 7 barcodes in the library. Barcoded cells are indicated by dashed-line shapes in the DAPI panels for all rounds, but only in the SBS panels corresponding to barcode signal for each SBS round. Scale bar for all sub-panels is 20 μm.

FIGS. 10A-10C depict microscopy images of an FFPE tissue section from a HepG2 cell line-derived xenograft tumor mouse model with 9 unique CAR designs following barcode amplification with a T7 RNA polymerase, reverse transcription, cDNA amplification, and in situ SBS. FIG. 10A depicts DAPI signal from the whole tumor with a 2 mm scale bar. FIG. 10B depicts tissue comprising the boxed region in FIG. 10A with DAPI signal overlayed with SBS signal from all nucleotides (A,C,G,T) in round 1 with a 75 μm scale bar. FIG. 10C depicts tissue comprising the boxed region in FIG. 10B with signal from each fluorescent channel in each round of SBS to depict the barcode readouts over multiple rounds. Dashed-line shapes indicate four individual cells in this field-of-view with distinct amplified barcodes, each corresponding to one of the 9 barcodes in the library. Barcoded cells are indicated by dashed-line shapes in the DAPI panels for all rounds, but only in the SBS channel corresponding to barcode signal for each SBS round. Scale bar for all sub-panels is 20 μm.

FIGS. 11A-11C depict microscopy images of an FFPE tissue section from a Hep G2 cell line-derived xenograft tumor mouse model with 56 unique CAR designs, including both armored and unarmored CAR constructs, following barcode amplification with a T7 RNA polymerase, reverse transcription, cDNA amplification, and in situ SBS. FIG. 11A depicts DAPI signal from the whole tumor with a 2 mm scale bar. FIG. 11B depicts Tissue comprising the boxed region in panel A with DAPI signal overlayed with SBS signal from all nucleotides (A,C,G,T) in round 1 with a 100 μm scale bar. FIG. 11C depicts Tissue comprising the boxed region in panel B with signal from each fluorescent channel in each round of SBS to depict the barcode readouts over multiple rounds. Dashed-line shapes indicate three individual cells in this field-of-view with distinct amplified barcodes, each corresponding to one of the 56 barcodes in the library. Barcoded cells are indicated by dashed-line shapes in the DAPI panels for all rounds, but only in the SBS panels corresponding to barcode signal for each SBS round. Scale bar for all sub-panels is 20 μm.

FIGS. 12A-12C depict microscopy images of an FFPE tissue section from an AsPC-1 cell line-derived xenograft tumor mouse model with 80 unique CAR designs, including both armored and unarmored CAR constructs, following barcode amplification with a T7 RNA polymerase, reverse transcription, cDNA amplification, and in situ SBS. FIG. 12A depicts DAPI signal from the whole tumor with a 1 mm scale bar. FIG. 12B depicts Tissue comprising the boxed region in FIG. 12A with DAPI signal overlayed with SBS signal from all nucleotides (A,C,G,T) in round 1 with a 100 μm scale bar. FIG. 12C depicts Tissue comprising the boxed region in FIG. 12B with signal from each fluorescent channel in each round of SBS to depict the barcode readouts over multiple rounds. Dashed-line shapes indicate two individual cells in this field-of-view with distinct amplified barcodes, each corresponding to one of the 80 barcodes in the library. Barcoded cells are indicated by dashed-line shapes in the DAPI panels for all rounds, but only in the SBS panels corresponding to barcode signal for each SBS round. Scale bar for all sub-panels is 20 μm.

FIGS. 13A-13C depict microscopy images of an FFPE tissue section from an AsPC-1 cell line-derived xenograft tumor mouse model with 10 unique CAR and shRNA designs, following barcode amplification with a T7 RNA polymerase, reverse transcription, cDNA amplification, and in situ SBS. FIG. 13A depicts DAPI signal from the whole tumor with a 1 mm scale bar. FIG. 13B depicts Tissue comprising the boxed region in FIG. 13A with DAPI signal overlayed with SBS signal from all nucleotides (A,C,G,T) in round 1 with a 100 μm scale bar. FIG. 13C depicts Tissue comprising the boxed region in FIG. 13B with signal from each fluorescent channel in each round of SBS to depict the barcode readouts over multiple rounds. Dashed-line shapes indicate three individual cells in this field-of-view with distinct amplified barcodes, each corresponding to one of the 10 barcodes in the library. Barcoded cells are indicated by dashed-line shapes in the DAPI panels for all rounds, but only in the SBS channel corresponding to barcode signal for each SBS round. Scale bar for all sub-panels is 20 μm.

FIGS. 14A-14C depicts quantification of barcodes detected in vivo from T cells transduced with the library of 9 barcoded CAR constructs shown in Table 1. Barcoded CAR construct Design Number is shown on the x-axis and the number of each design detected is shown on the y-axis. Panels depict barcodes detected in FFPE tissue sections containing CAR T cells made from T cell Donor 1 (FIG. 14A), T cell Donor 2 (FIG. 14B), and T cell Donor 3 (FIG. 14C). Barcode holdout (BH) designs that were not part of the CAR library are not highly detected, whereas barcodes contained in the 9 CAR library were detected at higher levels.

FIG. 15 depicts quantification of barcodes detected in vivo from T cells transduced with the library of 7 barcoded CAR constructs shown in Table 2. Barcoded Design Number is shown on the x-axis and number of each design detected in an FFPE tissue section from an orthotopic patient-derived xenograft tumor is shown on the y-axis. All 7 barcoded designs were detected.

FIGS. 16A and 16B depict quantification of barcodes detected from T cells transduced with the 56 barcoded CAR construct library shown in Table 3. Barcoded CAR Designs are designated as D-1 through D-56 (true barcodes) and Barcode Holdouts are designated as BH-1 through BH-13. FIG. 16A depicts Design Number is shown on the x-axis and number of each design detected in an FFPE tissue section from a Hep G2 cell line-derived xenografted tumor is shown on the y-axis. Barcodes from the 56 CAR design library were detected at higher levels than barcode holdouts. FIG. 16B depicts total raw counts of all true barcodes detected vs. all barcode holdouts detected in tissue section from panel A.

FIGS. 17A and 17B depict quantification of barcodes detected in vivo from T cells transduced with the library of 80 barcoded CAR design constructs shown in Table 4. Design Number is shown on the x-axis and number of each design detected on the y-axis. Panels depict barcodes detected in an FFPE tissue section from AsPC-1 cell line-derived xenografts containing library CAR T cells made from T cell Donor 1 (FIG. 17A) and T cell Donor 2 (FIG. 17B).

FIGS. 18A and 18B depict microscopy images of T cells in vitro that were transduced with the library of 9 barcoded CAR design constructs from Table 1 following barcode amplification with a T7 RNA polymerase, reverse transcription, cDNA amplification, and in situ SBS. FIG. 18A depicts DAPI signal from all cells in the field-of-view with a 100 μm scale bar. FIG. 18B depicts cells comprising the boxed region in FIG. 18A with signal from each fluorescent channel in each round of SBS to depict the barcode readouts over multiple rounds. Dashed-line shapes indicate four individual cells in this field-of-view with distinct amplified barcodes, each corresponding to one of the 9 barcodes in the library. Barcoded cells are indicated by dashed-line shapes in the DAPI panels for all rounds, but only in the SBS channel corresponding to barcode signal for each SBS round. Scale bar for all sub-panels is 20 μm.

FIGS. 19A and 19B depict microscopy images of T cells in vitro that were transduced with a library of 16 CAR design constructs (each cell includes 0, 1, 2, or more constructs per cell), each with a unique barcode following the T7 promoter following barcode amplification with a T7 RNA polymerase, reverse transcription, cDNA amplification, and in situ SBS. FIG. 19A depicts DAPI signal from all cells in the field-of-view with a 100 μm scale bar. FIG. 19B depicts cells comprising the boxed region in FIG. 19A with signal from each fluorescent channel in each round of SBS to depict the barcode readouts over multiple rounds. Square dashed-line shape indicates a single cell with one distinct amplified barcode and diamond and circle dashed-line shapes indicate a single cell with two distinct amplified barcodes. Barcoded cells are indicated by dashed-line shapes in the DAPI panels for all rounds, but only in the SBS channel corresponding to barcode signal for each SBS round. Scale bar for all sub-panels is 20 μm.

FIG. 20 depicts quantification of barcodes detected in vitro from T cells transduced with the library of 9 barcoded CAR design constructs from Table 1. Construct Design Number is shown on the x-axis and the number of each design detected is shown on the y-axis.

FIG. 21 depicts quantification of barcodes detected in vitro from T cells transduced with the library of 7 barcoded CAR constructs from Table 2. Construct Design Number is shown on the x-axis and the number of each design detected is shown on the y-axis.

FIGS. 22A and 22B depict quantification of barcodes detected in vivo from T cells transduced with the library of 9 barcoded CAR constructs shown in Table 1. FIG. 22A depicts barcode raw counts and FIG. 22B depicts barcode proportions. Donor number indicates barcodes detected in FFPE tissue sections containing CAR T cells made from each of 3 donors. True barcodes include CAR design numbers 1-8 for all 3 donors, and design 9A for Donor 1 and Donor 2, and design 9B for Donor 3. Barcode Holdouts include 60 negative control barcodes, BH #1-59 for all 3 donors and Design 9B for Donor 1 and Donor 2, and Design 9A for donor 3.

FIG. 23 depicts a schematic comparing in vivo barcode amplification using either an SP6 or T7 promoter.

FIGS. 24A-24F depicts microscopy images of FFPE embedded tissue sections from an orthotopic patient-derived xenograft tumor mouse model with 7 CAR constructs, each having a unique barcode following barcode amplification with indicated sequence specific RNA polymerase, reverse transcription, cDNA amplification, and in situ sequencing-by-synthesis (SBS). The CAR library contained 6 constructs with the barcode after the T7 promoter and one construct with the barcode after both the T7 and SP6 promoters. FIGS. 24A and 24D depict DAPI signal from whole tumor sections with 1 mm scale bar. FIGS. 24B and 24E depict tissue comprising the boxed regions in FIGS. 24A and 24D, respectively, with DAPI signal overlayed with SBS signal from all nucleotides (A,C,G,T) in round 1 with a 100 μm scale bar. FIGS. 24C and 24F depict tissue comprising the boxed regions in panels FIGS. 24B and 24E, respectively, with signal from each fluorescent channel in each round of SBS to depict the barcode readouts over multiple rounds. Dashed-line shapes indicate individual cells in these fields-of-view with distinct amplified barcodes. Shapes in FIG. 24C depict multiple barcodes from multiple constructs with the T7 promoter. Shapes in FIG. 24E depict one barcode from the one construct with the SP6 promoter. Barcoded cells are indicated by dashed-line shapes in the DAPI panels for all rounds, but only in the SBS channel corresponding to barcode signal for each SBS round. Scale bar for all sub-panels is 20 μm.

FIGS. 25A-25D depict microscopy images of an FFPE tissue section from a Hep G2 cell line-derived xenograft tumor mouse model. FIG. 25A depicts DAPI signal from the whole tumor with a 2 mm scale bar. FIG. 25B depicts issue comprising the boxed region in FIG. 25A with DAPI signal overlayed with SBS signal from all nucleotides (A,C,G,T) in round 1 with a 75 μm scale bar. FIG. 25C depicts tissue comprising the boxed region in FIG. 25B with signal from each fluorescent channel in each round of SBS depict the barcode readouts over multiple rounds. Dashed-line shapes indicate two individual cells in this field-of-view with distinct amplified barcodes, each corresponding to one of the 56 barcodes in the library. Barcoded cells are indicated by dashed-line shapes in the DAPI panels for all rounds, but only in the SBS panels corresponding to barcode signal for each SBS round. FIG. 25D depicts tissue region same as FIG. 25C, shows DAPI staining on the left, the barcode signal from all channels in Round 1 in the middle, and shows staining for protein markers on the right panels including CD8, Granzyme B, LAG3, Cytokeratin, and PDL1. Scale bar for all sub-panels is 20 μm.

FIGS. 26A-26D depict microscopy images of an FFPE tissue section from an AsPC-1 cell line-derived xenograft tumor mouse model. FIG. 26A depicts DAPI signal from whole tumor with a 2 mm scale bar. FIG. 26B depicts Tissue comprising the boxed region in panel A with DAPI signal overlayed with SBS signal from all nucleotides (A,C,G,T) in round 1 with a 75 μm scale bar. FIG. 26C depicts tissue comprising the boxed region in panel B with signal from each fluorescent channel in each round of SBS depict the barcode readouts over multiple rounds. Dashed-line shapes indicate two individual cells in this field-of-view with distinct amplified barcodes, each corresponding to one of the 80 barcodes in the library. Barcoded cells are indicated by dashed-line shapes in the DAPI panels for all rounds, but only in the SBS panels corresponding to barcode signal for each SBS round. FIG. 26D depicts Tissue region same as FIG. 26C, shows DAPI staining on the left, the barcode signal from all channels in Round 1 in the middle, and shows staining for protein markers on the right panels including CD8, Granzyme B, LAG3, Cytokeratin. Scale bar for all sub-panels is 20 μm.

FIG. 27 depicts a graph showing proportion of barcoded cells overlapping with cells positive for T-cell marker (CD45 positive) or cells negative for T-cell marker (CD45 negative) in an FFPE tissue section from an AsPC-1 cell line-derived xenograft tumor mouse model injected with a cell library comprised of 80 barcoded constructs. The x-axis shows CD45 cell staining status, and the y-axis shows the proportion of barcodes in the tumor section that were assigned to either a CD45 positive or CD45 negative cell.

FIGS. 28A-28D depict microscopy images of an FFPE tissue section from a Hep G2 cell line-derived xenograft tumor mouse model. FIG. 28A depicts DAPI signal from whole tumor with a 2 mm scale bar. FIG. 28B depicts tissue comprising the boxed region in panel A with DAPI signal overlayed with SBS signal from all nucleotides (A,C,G,T) in round 1 with a 100 μm scale bar. FIG. 28C depicts tissue comprising the boxed region in FIG. 28B that has undergone one round of SBS. The tissue was imaged after first round of SBS to read out the first nucleotide of the barcode in individual cells. The signal from each channel is shown separately to show the fluorescent readout of the individual barcodes. Dashed line shape indicates an individual cell with distinct SBS signal in the first round.

FIG. 28D depicts tissue from the same region as FIG. 28C, shows DAPI staining on the left, the barcode signal from all channels in Round 1 overlaid with DAPI staining in the middle, and shows CAR RNA staining overlaid with DAPI staining on the right panel. Scale bar for all sub-panels is 20 μm.

### DETAILED DESCRIPTION

### I. Definitions

The terms “nucleic acid,” “polynucleotides,” and “oligonucleotides” refer to biopolymers of nucleotides and, unless the context indicates otherwise, includes modified and unmodified nucleotides, and both DNA and RNA, and modified nucleic acid backbones. Nucleic acids are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage. Primers and oligonucleotides used in methods disclosed herein comprise nucleotides. In some embodiments, a nucleotide may comprise any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase.

The terms “amplify,” “amplifying,” “amplification reaction” and their variants, refer generally to any action or process whereby at least a portion of a nucleic acid molecule (referred to as a “template”) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule.

“Complementarity” or “complementary” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See, e.g., Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. If all nucleotides of a polynucleotide are capable of forming Watson-Crick pairing with the nucleotides at the corresponding positions of an anti-parallel polynucleotide, the two polynucleotides are said to be “reverse complements” of each other.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “subject” and “patient” refer to an organism that is the source of a sample that is interrogated by the methods described herein. Such organisms preferably include, but are not limited to, mammals (e.g., murines, simians, equines, bovines, porcines, canines, felines, and the like), and more preferably includes humans.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred. For the avoidance of doubt, when the term “about” is used prior to a list of numbers, the term “about” applies to each member of the list. For example, “about 1, 2, 3, 4, or 5” should be understood to mean “about 1, about 2, about 3, about 4, or about 5.”

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

In the present application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in certain embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

### II. Methods

Disclosed herein, in various embodiments, are methods for the detection of target nucleic acids (e.g., nucleic acid barcodes) within a biological sample via imaging.

The present disclosure relates, in general, to methods and compositions for detecting one or more nucleic acid sequences of interest (e.g., nucleic acid barcodes) in a biological sample in situ. Provided herein, for example, are methods and compositions for detecting multiple different analytes in a sample via imaging. In some embodiments, a group of nucleic acid barcodes is designed to have a minimum mutual Hamming distance of 6. In some embodiments, a group of nucleic acid barcodes is designed to have a minimum mutual minimal Hamming distance of 5. In some embodiments, a group of nucleic acid barcodes is designed to have a minimum mutual Hamming distance of 4. In some embodiments, a group of nucleic acid barcodes is designed to have a minimum mutual Hamming distance of 3. In some embodiments, a group of nucleic acid barcodes is designed to have a minimum mutual Hamming distance of 2. In some embodiments, a group of nucleic acid barcodes is designed to have a minimum mutual Hamming distance of 1.

In various embodiments, the present disclosure provides a method of determining the presence, absence, amount, or localization of a nucleic acid sequence of interest (e.g., a nucleic acid barcode) in a biological sample in situ. In some embodiments, the contemplated method may comprise, within a biological sample, (a) reacting a sequence-specific RNA polymerase with a DNA molecule comprising a nucleic acid sequence of interest operably linked to a promoter configured to promote transcription of the nucleic acid sequence of interest to generate an RNA molecule comprising a sequence complementary to the nucleic acid sequence of interest; (b) reacting the RNA molecule with a reverse transcriptase to generate a cDNA molecule comprising a copy of the nucleic acid sequence of interest; and (c) performing in situ sequencing (e.g., sequencing-by-synthesis) to visualize the nucleic acid sequence of interest.

In various embodiments, the present disclosure provides a method of determining the presence, absence, amount, and/or localization of a nucleic acid sequence of interest in one or more fixed mammalian cells within a biological sample in situ, the method comprising: (a) reverse transcribing with a DNA primer a target RNA comprising the nucleic acid sequence of interest in the one or more fixed mammalian cells to generate a first cDNA molecule comprising the nucleic acid sequence of interest, wherein the DNA primer comprises: (i) a 5′ nucleic acid sequence comprising a sequence-specific RNA polymerase promoter; and (ii) a 3′ nucleic acid sequence that is complementary to a portion of the target RNA flanking the nucleic acid sequence of interest, wherein the DNA primer hybridizes to the target RNA; wherein the first cDNA molecule comprises the sequence-specific RNA polymerase promoter operably linked to the nucleic acid sequence of interest; (b) reacting the first cDNA molecule with a sequence-specific RNA polymerase to generate an RNA transcript comprising the nucleic acid sequence of interest; (c) reacting the RNA transcript with a reverse transcriptase enzyme to generate a second cDNA molecule comprising the nucleic acid sequence of interest; and (d) in situ sequencing the second cDNA molecule to visualize the nucleic acid sequence of interest in the one or more fixed mammalian cells.

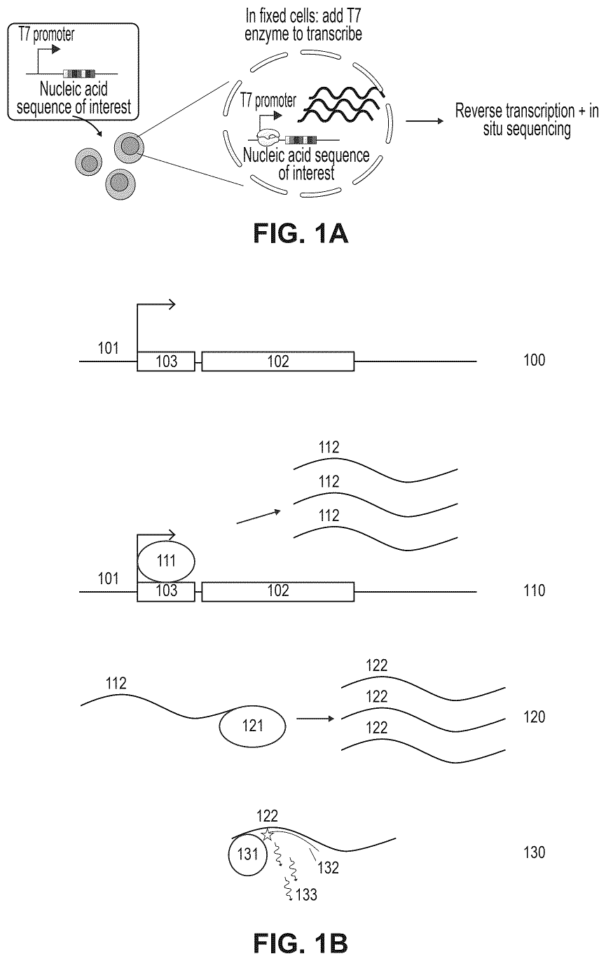

FIG. 1B illustrates an exemplary workflow of a method provided herein. At panel 100, a DNA molecule (101) comprising a nucleic acid sequence of interest (102) (e.g., a nucleic acid barcode) operably linked to a promoter (103) configured to promote transcription of the nucleic acid sequence of interest in the presence of a sequence-specific RNA polymerase is depicted within a fixed cell. At panel 110, the DNA molecule (101) is reacted with a sequence specific RNA polymerase (111) (e.g., a T7 RNA polymerase), thereby producing RNA molecules (112) that contain transcripts of the nucleic acid sequence of interest (102). At panel 120, an RNA molecule (112) is reacted with a reverse transcriptase (121), thereby producing cDNA molecules (122) that contain a copy of the nucleic acid sequence of interest. At panel 130, a cDNA molecule (122) undergoes sequencing-by-synthesis and is reacted with a sequencing polymerase (131) in the presence of detectably-labeled nucleotides to generate a short amplicon comprising detectably-labeled nucleotides (132) that produces a detectable signal (133). The detectable signal (133) can be removed and further rounds of detection of multiple nucleic acid sequences of interest can be performed.

In some embodiments, the DNA molecule is an exogenous DNA molecule. In some embodiments, the DNA molecule is an endogenous DNA molecule (e.g., genomic DNA or mitochondrial DNA). In some embodiments, the DNA molecule is derived from an exogenous nucleic acid sequence (e.g., introduced by viral transduction). In some embodiments, the DNA molecule is a cDNA molecule synthesized in situ from an exogenous RNA. In some embodiments, the DNA molecule is a cDNA molecule synthesized in situ from an endogenous RNA.

In various embodiments, the DNA molecule is generated by steps of: (i) contacting an RNA molecule comprising the nucleic acid sequence of interest with a DNA primer having a 5′ end and a 3′ end, wherein the DNA primer comprises: (A) a 3′ nucleic acid sequence that is complementary to a portion of the nucleic acid molecule flanking the nucleic acid sequence of interest, and (B) a 5′ nucleic acid sequence comprising the promoter, such that the DNA primer hybridizes to the nucleic acid sequence of interest; (ii) performing reverse transcription to extend the DNA primer, thereby generating a single-stranded cDNA; and (iii) using second strand synthesis to convert the single-stranded cDNA to double-stranded cDNA, thereby producing the DNA molecule.

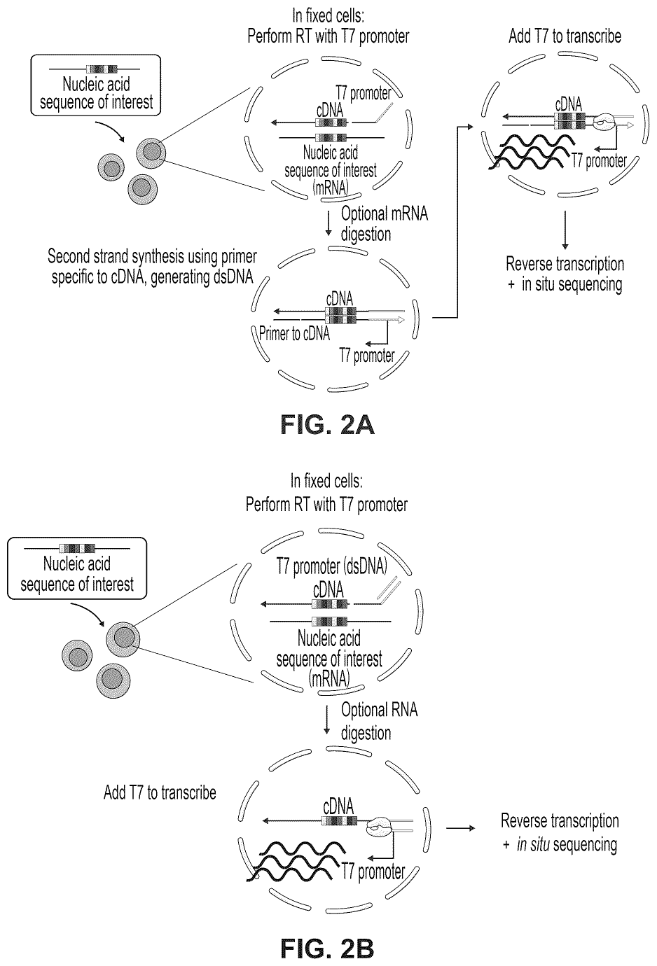

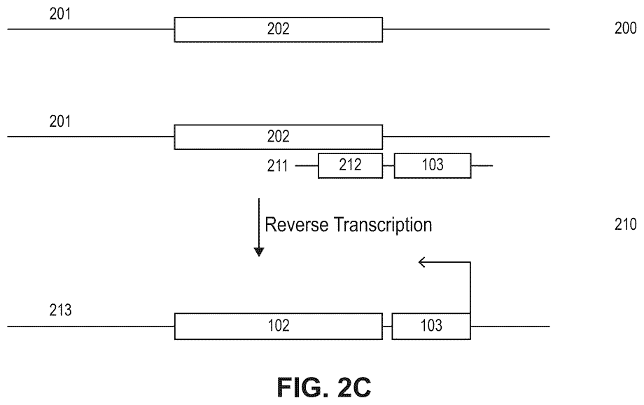

In certain embodiments, the nucleic acid sequence of interest is comprised within an RNA molecule (e.g., an mRNA) within a cell in the biological sample. FIG. 2C illustrates an exemplary workflow for the introduction of an exogenous promoter to a nucleic acid sequence within a cell. At panel 200, an RNA molecule (201) comprising a transcript of a nucleic acid sequence of interest (202) is depicted within a fixed cell in a biological sample. At panel 210, a DNA primer (211) comprising an exogenous sequence-specific promoter (103) linked to a nucleic acid sequence (212) that hybridizes with a portion of the transcript of the nucleic acid sequence of interest (202). A reverse transcription reaction is carried out, producing an ssDNA molecule (213) comprising the nucleic acid sequence of interest (102) operably linked to the exogenous sequence-specific promoter (103). The ssDNA molecule (213) can undergo an optional second-strand synthesis reaction to generate a dsDNA. The ssDNA molecule can also be synthesized using a DNA primer (211) comprising a double-stranded region comprising the sequence-specific promoter (103), facilitating RNA polymerase activity without a second-strand synthesis. The ssDNA molecule or the dsDNA molecule can then be introduced into the RNA synthesis, reverse transcription, and in situ sequencing process depicted in FIG. 1B.

In some embodiments, the method comprises, prior to reacting the DNA molecule with the sequence-specific RNA polymerase, contacting the cell with an RNase to degrade endogenous RNA molecules.

Persons skilled in the art will understand that over the course of successive rounds of RNA synthesis from a DNA template or reverse transcription, the nucleic acid sequence of interest may be present at certain steps as a reverse complement of the original nucleic acid sequence of interest. For example, an RNA synthesis reaction using a DNA template comprising the nucleic acid sequence of interest will produce a product RNA molecule comprising the reverse complement of the nucleic acid sequence of interest. A subsequent reverse transcription of the product RNA molecule will produce a cDNA molecule comprising the nucleic acid sequence that is a copy of the original.

The disclosed methods may detect the presence or absence, amount, or location of at least 100 nucleic acid sequences of interest, at least 90 nucleic acid sequences of interest, at least 80 nucleic acid sequences of interest, at least 70 nucleic acid sequences of interest, at least 60 nucleic acid sequences of interest, at least 50 nucleic acid sequences of interest, at least 40 nucleic acid sequences of interest, at least 30 nucleic acid sequences of interest, at least 20 nucleic acid sequences of interest, at least 15 nucleic acid sequences of interest, at least 10 nucleic acid sequences of interest, at least 9 nucleic acid sequences of interest, at least 8 nucleic acid sequences of interest, at least 7 nucleic acid sequences of interest, at least 6 nucleic acid sequences of interest, at least 5 nucleic acid sequences of interest, at least 4 nucleic acid sequences of interest, at least 3 nucleic acid sequences of interest, at least 2 nucleic acid sequences of interest, or at least 1 nucleic acid sequence of interest.

In some embodiments, multiple nucleic acid sequences of interest are present on a single nucleic acid molecule. In some embodiments, multiple nucleic acid sequences are present on more than one distinct nucleic acid molecules within the cell.

In certain embodiments, the sample is selected from a tissue sample, a liquid sample, and a cell sample. In some embodiments, the biological sample is a tissue sample. In some embodiments, the biological sample is a liquid sample. In some embodiments, the sample is a cell sample. In some embodiments, the sample is a two-dimensional cell culture sample. In some embodiments, the sample is a three-dimensional cell culture sample. In some embodiments, the sample is a suspension cell culture sample. In some embodiments, the sample is an organoid sample. In some embodiments, the sample is a heterogeneous cell culture sample. In some embodiments, the sample is a patient-derived cell sample. In some embodiments, the sample is a formalin-fixed paraffin-embedded (FFPE) tissue sample. In some embodiments, the sample is a cryopreserved tissue sample.

In certain embodiments, after the removal of the of the detectable signal, RNA synthesis, reverse transcription, and in situ sequencing is iteratively repeated 1 or more times, 2 or more times, 3 or more times, 4 or more times, 5 or more times, 6 or more times, 7 or more times, 8 or more times, 9 or more times, 10 or more times, 15 or more times, 20 or more times, 25 or more times, 30 or more times, 35 or more times, 40 or more times, 45 or more times, or 50 or more times. In some embodiments, RNA synthesis, reverse transcription, and in situ sequencing is iteratively repeated 1 or more times. In some embodiments, the removal of the detectable labels, re-probing, incorporation of labeled nucleotides, and re-imaging is iteratively repeated 2 or more times. In some embodiments, RNA synthesis, reverse transcription, and in situ sequencing is iteratively repeated 3 or more times. In some embodiments, RNA synthesis, reverse transcription, and in situ sequencing is iteratively repeated 4 or more times. In some embodiments, RNA synthesis, reverse transcription, and in situ sequencing is iteratively repeated 5 or more times. In some embodiments, RNA synthesis, reverse transcription, and in situ sequencing is iteratively repeated 6 or more times. In some embodiments, RNA synthesis, reverse transcription, and in situ sequencing is iteratively repeated 7 or more times. In some embodiments, RNA synthesis, reverse transcription, and in situ sequencing is iteratively repeated 8 or more times. In some embodiments, RNA synthesis, reverse transcription, and in situ sequencing is iteratively repeated 9 or more times. In some embodiments, RNA synthesis, reverse transcription, and in situ sequencing is iteratively repeated 10 or more times. In some embodiments, RNA synthesis, reverse transcription, and in situ sequencing is iteratively repeated 15 or more times. In some embodiments, RNA synthesis, reverse transcription, and in situ sequencing is iteratively repeated 20 or more times. In some embodiments, RNA synthesis, reverse transcription, and in situ sequencing is iteratively repeated 25 or more times. In some embodiments, RNA synthesis, reverse transcription, and in situ sequencing is iteratively repeated 30 or more times. In some embodiments, RNA synthesis, reverse transcription, and in situ sequencing is iteratively repeated 35 or more times. In some embodiments, RNA synthesis, reverse transcription, and in situ sequencing is iteratively repeated 40 or more times. In some embodiments, RNA synthesis, reverse transcription, and in situ sequencing is iteratively repeated 45 or more times. In some embodiments, RNA synthesis, reverse transcription, and in situ sequencing is iteratively repeated 50 or more times.

### Biological Samples

The systems and methods described herein may be used to detect the presence or absence, or to quantify the amount of one or more nucleic acid sequences of interest in a biological sample, e.g., a cell sample or a tissue sample.

Nucleic acid sequences of interest may be detected and/or quantified in a variety of samples. In certain embodiments, the sample is derived from a subject.

The sample can be in any form that allows for measurement of the nucleic acid sequence of interest. Therefore, the sample must be sufficient for processing to permit detection of the analyte, such as preparation of thin sections. Accordingly, the sample can be fresh, preserved through suitable cryogenic techniques, or preserved through non-cryogenic techniques.

In some embodiments, the sample is a body fluid sample, such as a blood, scrum, plasma, urine, saliva, cerebrospinal fluid, or interstitial fluid sample.

In various embodiments, the biological sample comprises one or more mammalian cells selected from stem cells, mesodermal cells, endodermal cells, ectodermal cells, cardiomyocytes, immune cells, epithelial cells, pneumocytes, club cells, paneth cells, pancreatic cells, stomach cells, goblet cells, gland cells, duct cells, centroacinar cells, brush border cells, endocrine cells, thyroid gland cells, pancreatic islet cells, mucous cells, pituitary cells, neurons, sensory neurons, receptor neurons, neuronal progenitors, cone cells, rod cells, interneurons, astrocytes, oligodendrocytes, ependymal cells, pituicytes, adipocytes, lipocytes, cells of the kidney or urinary system, reproductive cells, endothelial cells, extracellular matrix cells, contractile cells, skeletal muscle cells, cardiac muscle cells, blood cells, germ cells, nurse cells, or interstitial cells. In various embodiments, the biological sample comprises one or more immune cells. In some embodiments, the immune cells are T cells, NK cells, B cells, macrophages, dendritic cells, mast cells, monocytes, neutrophils, basophils, eosinophils, hematopoietic stem cells, or immortalized immune cells. In some embodiments, the immune cells are T cells, NK cells, or B cells, mast cells, dendritic cells, macrophages, neutrophils, basophils, and/or eosinophils. In some embodiments, the immune cells express one or more cell therapy constructs (e.g., an engineered immune receptor). In some embodiments the immune cells express a chimeric antigen receptor (CAR). In some embodiments, the immune cells are chimeric-antigen receptor-expressing T (CAR-T) cells or chimeric-antigen receptor-expressing NK (CAR-NK) cells. In some embodiments, the immune cells are chimeric-antigen receptor-expressing T (CAR-T) cells. In some embodiments, the immune cells are chimeric-antigen receptor-expressing NK (CAR-NK) cells. In some embodiments, the biological sample comprises embryonic stem cells (ESCs) or induced pluripotent stem cells (iPSCs) or cells derived from ESCs or iPSCs, and optionally differentiated to a specific lineage.

In some embodiments, the sample is a tissue sample, such as a biopsy sample. A biopsy sample can be obtained by using conventional biopsy instruments and procedures. Endoscopic biopsy, excisional biopsy, incisional biopsy, fine needle biopsy, punch biopsy, shave biopsy and skin biopsy are examples of recognized medical procedures that can be used by one of skill in the art to obtain tissue samples. A standard process for handling clinical biopsy tissue specimens is to fix the tissue sample in formalin and then embed the sample in paraffin. Samples in this form are commonly known as formalin-fixed, paraffin-embedded (FFPE) tissue. Suitable techniques of tissue preparation for subsequent analysis are well-known to those of skill in the art.

In certain embodiments, the sample is a cell sample, or a cell supernatant sample.

In some embodiments, the biological sample comprises one or more cells. In some embodiments, the biological sample comprises one or more cells consisting of cells from a single species. In some embodiments, the biological sample comprises one or more cells from multiple species. In some embodiments, the biological sample comprises human cells and mouse cells. In some embodiments, the biological sample comprises human immune cells and mouse cells. In some embodiments, the biological sample comprises one or more cancer cells or fibroblast cells. In some embodiments, the biological sample comprises one or more human cancer cells. In some embodiments, the biological sample comprises one or more murine cancer cells.

In various embodiments, the biological sample is fixed. In some embodiments, the biological sample is fixed using a solution comprising formaldehyde and/or paraformaldehyde. In some embodiments, the biological sample is fixed using a solution comprising greater than 1% paraformaldehyde (w/v). In some embodiments, the biological sample is fixed using a sample comprising 4% paraformaldehyde. In some embodiments, the sample is an FFPE tissue sample.

In some embodiments, the biological sample is fixed using cryofixation (e.g., using liquid nitrogen). In some embodiments, the biological sample fixed using cryopreservation comprises optimal cutting temperature (OCT) compound, a hydrogel matrix, or a swellable polymer hydrogel. In some embodiments, the sample fixed using cryopreservation comprises optimal cutting temperature (OCT) compound. In some embodiments, the sample comprises a hydrogel matrix. In some embodiments, the sample comprises a swellable polymer hydrogel.

In some embodiments, the sample is fixed using a solution comprising an alcohol. In some embodiments, the alcohol is methanol or ethanol. In some embodiments, the alcohol is methanol. In some embodiments, the alcohol is ethanol. In some embodiments, the sample is fixed using a solution comprising glutaraldehyde.

### Nucleic Acid Sequences

In certain embodiments, the methods disclosed herein relate to the determination of the presence, absence, amount, and/or localization of one or more nucleic acid sequences of interest. Examples of nucleic acids include DNA, mRNA, pre-mRNA, nascent RNA, transfer RNA, antisense oligonucleotides, siRNA, miRNA, tmRNA, snRNA, piRNA, sRNA, circular RNAs, snoRNA, CRNA, pRNA, sgRNA, gRNA (e.g., for CRISPR-mediated gene editing applications or CRISPR-mediated gene activating or silencing applications), manufactured DNA or RNA (e.g., for a therapeutic, screening or basic science purpose). In some embodiments, the nucleic acid sequence of interest is located on a complementary DNA (cDNA) molecule produced by reverse transcribing an RNA molecule comprising the nucleic acid sequence of interest.

In some embodiments, the nucleic acid sequence of interest is a nucleic acid barcode. A “nucleic acid barcode” or “barcode” (also referred to as a “unique molecular identifier” or “UMI”) can refer to random or pre-determined nucleotide sequences of defined length that can be used to identify specific cells or cell types. Nucleic acid barcodes have utility in numerous applications, including sequencing techniques. Additional details on nucleic acid barcoding can be found, for example in U.S. Pat. No. 8,053,192, PCT Publication No. WO 2005/068656, and U.S. Patent Publication No. 2013/0274117.

In various embodiments, the nucleic acid sequence of interest is located on an exogenous nucleic acid molecule or on a nucleic acid molecule derived from an exogenous nucleic acid sequence. In some embodiments, the exogenous nucleic acid is introduced into the one or more mammalian cells prior to fixation. Exogenous nucleic acids can be introduced to cells by any suitable method known in the art. In some embodiments, the exogenous nucleic acid is introduced into the one or more mammalian cells by viral transduction, site-specific nucleases, or site-specific recombinases. In some embodiments, the exogenous nucleic acid is introduced into the one or more mammalian cells by viral transduction. In some embodiments, the exogenous nucleic acid is introduced into the one or more mammalian cells by site-specific nucleases. In some embodiments, the exogenous nucleic acid is introduced into the one or more mammalian cells by site-specific recombinases. In some embodiments, the exogenous nucleic acid sequence is incorporated into the genome of one or more mammalian cells. In some embodiments, the exogenous nucleic acid is incorporated at a specific site (i.e., a pre-specified site) within the genome of the one or more mammalian cells. In some embodiments, the exogenous nucleic acid is incorporated at a random site in the genome of the one or more mammalian cells. In some embodiments, the exogenous nucleic acid is incorporated into the one or more mammalian cells using a viral vector. In some embodiments, the viral vector is selected from a lentiviral vector, a retroviral vector, an adenoviral vector, an HSV vector, a baculoviral vector, a virus-like particle, a pseudotyped virus-like capsid, an oncolytic viral vector, or an adeno-associated viral (AAV) vector. In some embodiments, the viral vector is a lentiviral vector. In some embodiments, the viral vector is a retroviral vector. In some embodiments, the viral vector is a adenoviral vector. In some embodiments, the viral vector is an HSV vector. In some embodiments, the viral vector is a baculoviral vector. In some embodiments, the viral vector is a virus-like particle. In some embodiments, the viral vector is an HSV vector. In some embodiments, the viral vector is an oncolytic viral vector. In some embodiments, the viral vector is a pseudotyped virus-like capsid. In some embodiments, the viral vector is an AAV vector.

In some embodiments, the exogenous nucleic acid is not integrated into a chromosome of the one or more mammalian cells. In some embodiments, the exogenous nucleic acid is not integrated into a chromosome of the one or more mammalian cells but is retained in a nucleus of the one or more mammalian cells. In some embodiments, the exogenous nucleic acid molecule is comprised within a plasmid or an artificial chromosome.

In various embodiments, the nucleic acid sequence of interest is an endogenous nucleic acid sequence. In some embodiments, the nucleic acid sequence of interest is an endogenous nucleic acid sequence that is operably linked to an exogenous promoter. In some embodiments, the nucleic acid sequence of interest is an endogenous nucleic acid sequence that is variable between cells within the biological sample. In some embodiments, the nucleic acid sequence of interest is an endogenous nucleic acid sequence that does not vary between cells within the biological sample. As provided for herein, the nucleic acid sequence of interest can contain more than sequence of interest.

In some embodiments, the nucleic acid sequence of interest is less than 100, less than 90, less than 80, less than 70, less than 60, less than 50, less than 40, less than 30, less than 25, less than 20, less than 15, less than 10, or less than 5 nucleotides in length. In some embodiments, the nucleic acid sequence of interest is less than 100 nucleotides in length. In some embodiments, the nucleic acid sequence of interest is less than 90 nucleotides in length. In some embodiments, the nucleic acid sequence of interest is less than 80 nucleotides in length. In some embodiments, the nucleic acid sequence of interest is less than 70 nucleotides in length. In some embodiments, the nucleic acid sequence of interest is less than 60 nucleotides in length. In some embodiments, the nucleic acid sequence of interest is less than 50 nucleotides in length. In some embodiments, the nucleic acid sequence of interest is less than 40 nucleotides in length. In some embodiments, the nucleic acid sequence of interest is less than 30 nucleotides in length. In some embodiments, the nucleic acid sequence of interest is less than 25 nucleotides in length. In some embodiments, the nucleic acid sequence of interest is less than 20 nucleotides in length. In some embodiments, the nucleic acid sequence of interest is less than 15 nucleotides in length. In some embodiments, the nucleic acid sequence of interest is less than 10 nucleotides in length. In some embodiments, the nucleic acid sequence of interest is less than 5 nucleotides in length.

In some embodiments, the nucleic acid sequence is present in all or substantially all cells within the biological sample. In some embodiments, the nucleic acid sequence of interest is present within less than 100% of the cells within the biological sample. In some embodiments, the nucleic acid sequence of interest is present within less than 90% of the cells within the biological sample. In some embodiments, the nucleic acid sequence of interest is present within less than 80% of the cells within the biological sample. In some embodiments, the nucleic acid sequence of interest is present within less than 70% of the cells within the biological sample. In some embodiments, the nucleic acid sequence of interest is present within less than 60% of the cells within the biological sample. In some embodiments, the nucleic acid sequence of interest is present within less than 50% of the cells within the biological sample. In some embodiments, the nucleic acid sequence of interest is present within less than 40% of the cells within the biological sample. In some embodiments, the nucleic acid sequence of interest is present within less than 30% of the cells within the biological sample. In some embodiments, the nucleic acid sequence of interest is present within less than 20% of the cells within the biological sample. In some embodiments, the nucleic acid sequence of interest is present within less than 10% of the cells within the biological sample.

In some embodiments, the nucleic acid sequence of interest is comprised within a nucleic acid molecule further comprising a nucleic acid sequence encoding one or more proteins. In some embodiments, the DNA molecule further comprises one or more polynucleotide sequences encoding exogenous proteins, endogenous proteins, or a mixture of exogenous and endogenous proteins. In some embodiments, the one or more proteins comprise one or more exogenous proteins. In some embodiments, the one or more exogenous proteins have expression controlled by one or more proteins endogenous to the mammalian cells.

In some embodiments, the one or more exogenous proteins are synthetic proteins and/or chimeric proteins. In some embodiments, the one or more exogenous proteins are independently selected from the group consisting of a chimeric antigen receptor (CAR), an antibody, a T-cell receptor, a cytokine, a cell-surface receptor, a transcription factor, a signaling protein, and a protease. In some embodiments, the one or more exogenous proteins comprise a CAR. In some embodiments, the one or more exogenous proteins comprise an antibody. In some embodiments, the one or more exogenous proteins comprise an antibody. In some embodiments, the one or more exogenous proteins comprise a T-cell receptor. In some embodiments, the one or more exogenous proteins comprise a cytokine. In some embodiments, the one or more exogenous proteins comprise a cell-surface receptor. In some embodiments, the one or more exogenous proteins comprise a transcription factor. In some embodiments, the one or more exogenous proteins comprise a signaling protein. In some embodiments, the one or more exogenous proteins comprise a protease.

In some embodiments, two or more exogenous proteins are expressed. In some embodiments, three or more exogenous proteins are expressed. In some embodiments, four or more exogenous proteins are expressed. In some embodiments, five or more exogenous proteins are expressed. In some embodiments, six or more exogenous proteins are expressed. In some embodiments, seven or more exogenous proteins are expressed. In some embodiments, eight or more exogenous proteins are expressed. In some embodiments, nine or more exogenous proteins are expressed. In some embodiments, ten or more exogenous proteins are expressed.

In some embodiments, the nucleic acid sequence of interest is comprised within a nucleic acid molecule further comprising a nucleic acid sequence encoding an endogenous protein. In some embodiments, the nucleic acid sequence of interest is comprised within a nucleic acid molecule further comprising a nucleic acid sequence encoding an exogenous RNA. In some embodiments, the nucleic acid sequence of interest is comprised within a nucleic acid molecule further comprising a nucleic acid sequence encoding an endogenous RNA.

In some embodiments, the nucleic acid sequence of interest is comprised within a nucleic acid molecule further comprising a nucleic acid sequence encoding a viral genome. In some embodiments, the viral genome is an oncolytic viral genome. In some embodiments, the oncolytic viral genome is of an adenovirus, a herpes simplex virus (HSV), a parvovirus, or a poxvirus (e.g., vaccinia virus or myxoma virus)

In some embodiments, the DNA molecule further comprises a polynucleotide sequence encoding a nucleic acid sequence that alters expression, function, and/or sequence of one or more genes. In some embodiments, the DNA molecule further comprises a polynucleotide sequence encoding a nucleic acid sequence that contributes to altering the genome of the cell. In some embodiments, the altering the genome of the cell comprises introducing one or more mutations in the genome of the cell. In some embodiments, the one or more mutations comprise one or more of nucleic acid insertions, nucleic acid deletions, frameshift mutations, missense mutations, nonsense mutations. In some embodiments, the target RNA encodes for a sequence that can alter expression of one or more genes in isolation or as a component of a gene editing system (e.g. CRISPR). In some embodiments, the nucleic acid sequence that alters expression, function, and/or sequence of one or more genes is selected from the group consisting of an sgRNA (e.g., in a CRISPR construct), a gRNA, an shRNA, and an miRNA. In some embodiments, the nucleic acid sequence that alters expression of one or more genes is an sgRNA. In some embodiments, the nucleic acid sequence that alters expression, function, and/or sequence of one or more genes is a gRNA. In some embodiments, the nucleic acid sequence that alters expression, function, and/or sequence of one or more genes is an shRNA. In some embodiments, the nucleic acid sequence that alters expression, function, and/or sequence of one or more genes is an miRNA.

### Promoters and Sequence-Specific RNA Polymerases

In various embodiments, the methods disclosed herein involve reacting a DNA molecule comprising a sequence-specific RNA polymerase promoter operably linked to a nucleic acid sequence of interest with a sequence-specific RNA polymerase to drive transcription of the nucleic acid sequence of interest within one or more mammalian cells within a biological sample. A sequence-specific RNA polymerase and promoter can be employed to selectively amplify one or more target nucleic acid sequences of interest with minimal or substantially no off-target RNA synthesis. In some embodiments, the methods disclosed herein comprise reacting the biological samples with an RNase (e.g., to degrade endogenous RNA) prior to addition of the sequence-specific RNA polymerase.

In some embodiments, the one or more mammalian cells comprise a single nucleic acid sequence of interest operably linked to a sequence-specific RNA polymerase promoter configured to drive transcription of the nucleic acid sequence of interest in the presence of a sequence-specific RNA polymerase. In some embodiments, the one or more mammalian cells comprise multiple nucleic acid sequences of interest, each operably linked to a sequence-specific RNA polymerase promoter configured to drive transcription of the respective nucleic acid sequences of interest in the presence of a sequence-specific RNA polymerase. In some embodiments, each sequence-specific RNA polymerase and promoter is identical. In some embodiments, each sequence-specific RNA polymerase and promoter is different. In some embodiments, some sequence-specific RNA polymerases and promoters are identical and some are different.

In some embodiments, the one or more mammalian cells comprise a first nucleic acid sequence of interest operably linked to a first sequence-specific RNA polymerase promoter configured to drive transcription of the first nucleic acid sequence of interest in the presence of a first sequence-specific RNA polymerase and a second nucleic acid sequence of interest operably linked to a second sequence-specific RNA polymerase promoter configured to drive transcription of the nucleic acid sequence of interest in the presence of a second sequence-specific RNA polymerase. In some embodiments, the first and second promoters and RNA polymerases are the same. In some embodiments, the first and second promoters and RNA polymerases are different.

In some embodiments, the one or more mammalian cells comprise three or more, four or more, five or more, six or more, seven or more, eight or more, nine or more, or ten or more sequence-specific RNA polymerase promoters configured to each drive transcription of a distinct nucleic acid sequence of interest in the presence of a distinct sequence-specific RNA polymerase. In some embodiments, the one or more mammalian cells comprise three or more sequence-specific RNA polymerase promoters configured to each drive transcription of a distinct nucleic acid sequence of interest in the presence of a distinct sequence-specific RNA polymerase. In some embodiments, the one or more mammalian cells comprise four or more sequence-specific RNA polymerase promoters configured to each drive transcription of a distinct nucleic acid sequence of interest in the presence of a distinct sequence-specific RNA polymerase. In some embodiments, the one or more mammalian cells comprise five or more sequence-specific RNA polymerase promoters configured to each drive transcription of a distinct nucleic acid sequence of interest in the presence of a distinct sequence-specific RNA polymerase. In some embodiments, the one or more mammalian cells comprise six or more sequence-specific RNA polymerase promoters configured to each drive transcription of a distinct nucleic acid sequence of interest in the presence of a distinct sequence-specific RNA polymerase. In some embodiments, the one or more mammalian cells comprise seven or more sequence-specific RNA polymerase promoters configured to each drive transcription of a distinct nucleic acid sequence of interest in the presence of a distinct sequence-specific RNA polymerase. In some embodiments, the one or more mammalian cells comprise eight or more sequence-specific RNA polymerase promoters configured to each drive transcription of a distinct nucleic acid sequence of interest in the presence of a distinct sequence-specific RNA polymerase. In some embodiments, the one or more mammalian cells comprise nine or more sequence-specific RNA polymerase promoters configured to each drive transcription of a distinct nucleic acid sequence of interest in the presence of a distinct sequence-specific RNA polymerase. In some embodiments, the one or more mammalian cells comprise ten or more sequence-specific RNA polymerase promoters configured to each drive transcription of a distinct nucleic acid sequence of interest in the presence of a distinct sequence-specific RNA polymerase.

In some embodiments, the RNA polymerase is a DNA-dependent RNA polymerase. In some embodiments, the sequence-specific RNA polymerase promoter is a transcriptionally active variant of a known sequence-specific RNA polymerase promoter (i.e., a promoter comprising a mutant sequence relative to the known sequence but having transcriptional activity). In some embodiments, the sequence-specific RNA polymerase promoter is a phage promoter or a transcriptionally active variant thereof and the sequence-specific RNA polymerase is a phage RNA polymerase (i.e., a promoter derived from a bacteriophage). In some embodiments, the sequence-specific RNA polymerase promoter and the sequence-specific RNA polymerase are selected from the group consisting of: a T7 promoter or a transcriptionally active variant thereof and a T7 RNA polymerase, respectively; a T3 promoter or a transcriptionally active variant thereof and a T3 RNA polymerase, respectively; and an SP6 promoter or a transcriptionally active variant thereof and an SP6 RNA polymerase, respectively. In some embodiments, the promoter is a T7 promoter or a transcriptionally active variant thereof and the RNA polymerase is a T7 RNA polymerase. In some embodiments, the promoter is a T3 promoter or a transcriptionally active variant thereof and the RNA polymerase is a T3 RNA polymerase. In some embodiments, the promoter is an SP6 promoter or a transcriptionally active variant thereof and the RNA polymerase is an SP6 RNA polymerase.

In some embodiments, the sequence-specific RNA polymerase promoter is a bacterial promoter or a transcriptionally active variant thereof and the sequence-specific RNA polymerase is a bacterial RNA polymerase. In some embodiments, the sequence-specific RNA polymerase promoter is a eukaryotic promoter or a transcriptionally active variant thereof and the sequence-specific RNA polymerase is a eukaryotic RNA polymerase. In some embodiments, the sequence-specific RNA polymerase promoter is a viral promoter or a transcriptionally active variant thereof and the sequence-specific RNA polymerase is a viral RNA polymerase. In some embodiments, the sequence-specific RNA polymerase promoter is a synthetic promoter and the sequence-specific RNA polymerase is a synthetic RNA polymerase.

In some embodiments, the sequence specific promoter is comprised within an exogenous nucleic acid molecule or a nucleic acid molecule derived from an exogenous nucleic acid molecule. In some embodiments, the exogenous nucleic acid molecule further comprises a transcriptional terminator. In some embodiments, the transcriptional terminator is a T7 transcriptional terminator, a T3 transcriptional terminator, or an SP6 transcriptional terminator. In some embodiments, the transcriptional terminator is a T7 transcriptional terminator. In some embodiments, the transcriptional terminator is a T3 transcriptional terminator. In some embodiments, the transcriptional terminator is an SP6 transcriptional terminator.

In some embodiments, the sequence specific promoter is an exogenous promoter (e.g., a T7 promoter) that is introduced into the cell to control expression of an endogenous nucleic acid sequence of interest. In some embodiments, the exogenous promoter is introduced by (1) introducing into the one or more mammalian cells a DNA primer comprising (a) a 5′ nucleic acid sequence comprising the exogenous promoter, and (b) a 3′ nucleic acid sequence that is complementary to a portion of a target RNA that comprises a nucleic acid sequence of interest, and (2) driving a reverse transcription reaction to synthesize a cDNA molecule comprising the exogenous primer operably linked to the nucleic acid sequence of interest. In some embodiments, a second strand synthesis reaction is performed to convert the cDNA molecule to double-stranded DNA (dsDNA) (FIG. 2A). In some embodiments, a second strand synthesis reaction is not performed and the DNA primer comprises a dsDNA portion comprising the exogenous promoter, such that a reverse transcription reaction of the cDNA molecule can be directly driven using the cDNA (FIG. 2B). In some embodiments, the dsDNA portion of the DNA primer is hybridized dsDNA or a hairpin. In some embodiments, the dsDNA portion of the DNA primer is hybridized dsDNA. In some embodiments, the dsDNA portion of the DNA primer is a hairpin.

In some embodiments, the exogenous promoter is integrated into the genome of a cell. In some embodiments, the exogenous promoter is integrated at a specific site in the genome of the cell. In some embodiments, the exogenous promoter is incorporated at a random site in the genome of the one or more mammalian cells. In some embodiments, the exogenous promoter is integrated into the genome of the cell by site-specific nucleases. In some embodiments, the exogenous promoter is integrated into the genome of the cell by site-specific recombinases.

In some embodiments, more than one distinct exogenous promoter is integrated into the genome of the cell. In some embodiments, each exogenous promoter is incorporated at a specific site in the genome of the cell. In some embodiments, some exogenous promoters are incorporated at specific sites in the genome of the cell and other exogenous promoters are incorporated at random sites in the genome of the cell.

In some embodiments, the exogenous promoter is a sequence-specific RNA polymerase promoter. In some embodiments, the exogenous promoter is a phage promoter. In some embodiments, the exogenous promoter is selected from the group consisting of: a T7 promoter, a T3 promoter; and an SP6 promoter. In some embodiments, the exogenous promoter is a T7 promoter. In some embodiments, the exogenous promoter is a T3 promoter. In some embodiments, the exogenous promoter is an SP6 promoter.

### Reverse Transcription

In some embodiments, the methods disclosed herein involve performing a reverse transcription reaction to generate a cDNA molecule of the transcripts produced by the sequence-specific RNA polymerases. Reverse transcription generally involves reacting an RNA molecule and a DNA primer with a reverse transcriptase enzyme to produce a cDNA molecule. Reverse transcription cam be carried out using any suitable method known in the art. In some embodiments, the DNA primer is a random primer. In some embodiments, the DNA primer is a sequence-specific primer. In some embodiments, the reverse transcriptase is an AMV reverse transcriptase. In some embodiments, the reverse transcriptase is an MMLV reverse transcriptase. In some embodiments, the reverse transcriptase is an engineered reverse transcriptase.

### In Situ Sequencing

In various embodiments, the methods disclosed herein comprise detection of target nucleic acids by one or more in situ sequencing techniques (e.g., sequencing-by-synthesis, sequencing-by-ligation, or sequencing-by-avidity). In situ sequencing techniques generally involve incorporation of nucleotides or oligonucleotides comprising a detectable label (for example, a fluorescent dye comprising a fluorophore) into a nucleic acid that is complementary to a template nucleic acid (e.g., a nucleic acid sequence of interest).

In some embodiments, the nucleic acid sequence can be detected via sequencing-by-synthesis. Sequencing-by-synthesis can be performed with an enzyme having DNA polymerase activity. Sequencing-by-synthesis is typically performed using an enzyme with DNA polymerase activity that incorporates one or more labeled nucleotides, wherein the labeled nucleotide comprises a detectable label (e.g., a fluorescent label) and, optionally, a cleavable chain terminator modification. In some embodiments, the detectable label is a cleavable detectable label. In each round, either a mixed population of multiple nucleotides, or a population comprising a single nucleotide type, is incorporated onto free 3′ ends of target nucleic acids to be sequenced. Imaging is performed to identify the detectable labels added to the target nucleic acids, label removal is performed (e.g., cleavage, photobleaching, dissociation via changing buffer conditions, etc.), and another round of nucleotides are added to target nucleic acids.

In some embodiments, nucleotides comprising chain terminator modifications are also incorporated into the probe to prevent the incorporation of additional nucleotides. Preventing the incorporation of additional nucleotides in a first set of probes can be useful to allow the labeling and detection of additional analytes in successive rounds by preventing the incorporation of labeled nucleotides into previously detected probes. In some embodiments, the chain terminator is irreversible. In some embodiments, the chain terminator is reversible. In some embodiments, the chain terminator is present in the nucleotide comprising the detectable label. In some embodiments, the chain terminator is present in an additional nucleotide that does not comprise a detectable label.

In some embodiments, sequencing-by-ligation can be performed using oligonucleotides containing degenerate bases (e.g., via an oligonucleotide pool), and incorporated using a ligation reaction. Sequencing-by-ligation can be performed using labeled oligonucleotide species (often a pool, e.g., with degenerate bases except at the site(s) to be sequenced), and incorporation into the analyte is facilitated using an enzyme with DNA ligase activity, unlike sequencing-by-synthesis. Cleavable detectable labels and reversible chain terminators can both still be used, similar to sequencing-by-synthesis. To sequence into the degenerate bases within the oligonucleotide, the anchor sequence and ligated oligonucleotides can be removed from the analyte, and a new set incorporated in a subsequent round.

In some embodiments, sequencing-by-avidity can be performed by binding detectably labeled polymer-nucleotide substrates (“avidites”) each comprising multiple copies of a single nucleotide to a DNA template. An engineered DNA polymerase binds to the template DNA and facilitates specific binding of the avidites to the cognate nucleotides without incorporating the avidites into the template DNA or synthesizing a complementary strand. Detectably labeled avidites can be subsequently visualized and then removed by washing, allowing for subsequent rounds of detection. In some embodiments, the detectable label on an avidite is a cleavable detectable label. Additional details on sequencing-by-avidity can be found in Arslan, S. et al. Sequencing by avidity enables high accuracy with low reagent consumption. Nat Biotechnol (2023).

In some embodiments, the methods disclosed herein comprise a rolling circle amplification reaction performed on the cDNA molecule comprising the nucleic acid sequence of interest prior to in situ sequencing. Information on rolling circle amplification is provided in Schweitzer, et al. PNAS. 2000 Aug. 29; 97 (18): 10113-9.

In some embodiments, the nucleic acid sequence of interest is flanked by a first padlock-binding sequence and a second padlock-binding sequence. In some embodiments, the method disclosed herein comprises the steps of (1) contacting the cDNA with a first padlock probe comprising a 5′ end and a 3′ end, wherein the first padlock probe comprises a 5′ nucleic acid sequence which is reverse complementary to the first padlock-binding site and a 3′ nucleic acid sequence which is reverse complementary to the second padlock-binding site, thereby allowing the 5′ and 3′ nucleic acid sequences to hybridize to the cDNA; (2) extending the 3′ end of the first padlock probe through the nucleic acid sequence of interest using a DNA polymerase; (3) ligating the 5′ end of the padlock probe to the extended 3′ end of the padlock probe, thereby generating a circular DNA template comprising a nucleic acid sequence reverse complementary to the nucleic acid sequence of interest; and (4) using rolling circle amplification of the DNA template to generate additional copies of the nucleic acid sequence of interest.

In some embodiments, multiple nucleic acid sequences of interest are each flanked by padlock-binding sites. In some embodiments, the pairs of padlock binding sites flanking each nucleic acid sequence of interest are identical. In some embodiments, the pairs of padlock binding sites flanking each nucleic acid sequence of interest are different. In some embodiments, a first nucleic acid sequence of interest is flanked by a first padlock-binding sequence and a second padlock-binding sequence and a second nucleic acid sequence of interest is flanked by a third padlock-binding sequence and a fourth padlock-binding sequence. In some embodiments, the first and second padlock-binding sequences are identical to the third and fourth padlock-binding sequences. In some embodiments, the first and second padlock-binding sequences are different from the third and fourth padlock-binding sequences. In some embodiments, the method disclosed herein comprises (1) contacting the cDNA with a second padlock probe comprising a 5′ end and a 3′ end, wherein the second padlock probe comprises a 5′ nucleic acid sequence which is reverse complementary to the third padlock-binding site and a 3′ nucleic acid sequence which is reverse complementary to the fourth padlock-binding site; (2) extending the 3′ end of the second padlock probe through the second nucleic acid sequence of interest using a DNA polymerase; (3) ligating the 5′ end of the second padlock probe to the extended 3′ end of the second padlock probe, thereby generating a circular DNA template comprising a nucleic acid sequence reverse complementary to the second nucleic acid sequence of interest; and (4) using rolling circle amplification of the DNA template to generate additional copies of the second nucleic acid sequence of interest.

### Detectable Labels

In some embodiments, the detectable labels used in the present invention comprise a fluorescent dye. Fluorescent dyes are widely used in biological research and medical diagnostics. In particular, a diversity of fluorophores with a distinguishable color range has made it more practical to perform multiplexed assays capable of detecting multiple biological targets at the same time. The ability to visualize multiple targets in parallel is often required for delineating the spatial and temporal relationships amongst different biological targets in vitro and in vivo.

In some embodiments, the fluorescent dye is an Alexa Fluor. Examples of Alexa Fluors include, but are not limited to Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, and Alexa Fluor 790.

In some embodiments, the fluorescent dye is a rhodamine dye. Examples of rhodamine dyes include, but are not limited to rhodamine, rhodamine 6G, rhodamine 123, rhodamine B, sulforhodamine 101, and sulforhodamine B.

In some embodiments, the fluorescent dye is a DyLight Fluor. Examples of DyLight Fluors include, but are not limited to DyLight 350, DyLight 405, DyLight 488, DyLight 550, DyLight 594, DyLight 633, DyLight 650, DyLight 680, DyLight 755, and DyLight 800.

In some embodiments, the fluorescent dye is a cyanine dye. Examples of cyanine dyes include, but are not limited to cyanine 2 (Cy2), cyanine 3 (Cy3), cyanine 3B (Cy3B), cyanine 3.5 (Cy3.5), cyanine 5 (Cy5), cyanine 5.5 (Cy5.5), cyanine 7 (Cy7), and cyanine 7.5 (Cy7.5).

In some embodiments, the fluorescent dye is an ATTO dye. Examples of ATTO dyes include, but are not limited to ATTO 390, ATTO 425, ATTO 465, ATTO 488, ATTO 495, ATTO 514, ATTO 520, ATTO 532, ATTO Rho6G, ATTO 540Q, ATTO 550, ATTO 565, ATTO Rho3B, ATTO Rho11, ATTO Rho12, ATTO Thio12, ATTO 580Q, ATTO Rho101, ATTO 590, ATTO Rho13, ATTO 594, ATTO 610, ATTO 612Q, ATTO 620, ATTO Rho14, ATTO 633, ATTO 647 ATTO 647N, ATTO 655, ATTO Oxa12, ATTO 665, ATTO 680, ATTO 700, ATTO 725, ATTO 740, and ATTO MB2.

Other examples of fluorescent dyes include, but are not limited to Freedom Dyes, Janelia Fluor Dyes, green fluorescent protein (GFP), red fluorescent protein (RFP), yellow fluorescent protein (YFP), blue fluorescent protein (BFP), cyan fluorescent protein (CFP), DSRed, cGFP, mEmerald, mWasabi, Azami Green, mAzurite, mCerulean, mTurquoise, mTopaz, mVenus, mCitrine, mBanana, Kusabia Orange, mOrange, dTomato, mTangerine, mRuby, mApple, mStrawberry, mCherry, mRaspberry, mPlum, fluorescein, phycoerythrin (PE), and peridinin chlorophyll protein (PerCP).

Fluorescent dyes can be detected by any suitable method known to those of ordinary skill in the art, preferably by fluorescence microscopy. In some embodiments, the fluorescence microscopy is wide-field fluorescence microscopy. In some embodiments, the fluorescence microscopy is laser scanning confocal microscopy. In some embodiments, the fluorescence microscopy is spinning disc confocal microscopy. In some embodiments, the fluorescence microscopy is two-photon microscopy. Methods of fluorescence microscopy are summarized in Sanderson et al., Cold Spring Harb Protoc. 2014 October; 2014 (10).

In some embodiments, the detectable labels of the invention can comprise a radioisotope. Detectable labels can either incorporate the label directly or indirectly by incorporating the label through a chelating agent, where the chelating agent has been incorporated into the compound). Furthermore, a label can be included as an additional substituent (group, moiety, position) to a compound of the invention or as an alternative substituent for any substituents that are present. Exemplary radioisotopes can include ³H, ¹¹C, ¹⁴C, ¹⁸F, ³²P, ³⁵S, ¹²³I, ¹²⁵I, ¹³¹I, ¹²⁴I, ¹⁹F, ⁷⁵Br, ¹³C, ¹³N, ¹⁵O, ⁷⁶Br, or ⁹⁹Tc. A radiolabel may appear at any substituent (group, moiety, position) on a compound or probe of the invention.

In various embodiments, a detectable label can be cleaved from the labeled nucleotide oligonucleotide, or avidite (i.e., cleavable detectable labels). In some embodiments, the detectable label is cleavable between the dye and the nitrogenous base. In some embodiments, the detectable label is cleavable between the dye and the sugar-phosphate backbone. In some embodiments, the cleavable detectable label is cleavable between the oligonucleotide and the labeled nucleotide. In some embodiments, cleavable detectable labels produce the same or similar detectable signal. In some embodiments, the cleavable detectable labels are fluorophores that have substantially overlapping excitation and/or emission spectra.

In some embodiments, the cleavable detectable label is cleaved by exposure to light. In some embodiments, the cleavable detectable label is cleaved by treatment with an acidic solution. In some embodiments, the cleavable detectable label is cleaved by treatment with an alkaline solution. In some embodiments, the cleavable detectable label is cleaved by treatment with a reducing agent. In some embodiments, the reducing agent is DTT or TCEP. In some embodiments, the cleavable detectable label is cleaved by treatment with a nuclease or DNA repair enzyme. In some embodiments, the cleavable detectable label is cleaved by treatment with palladium.

In some embodiments, the signal from a detectable label can be removed by incubation with a quenching agent. Examples of quenching agents include, but are not limited to Iowa Black, dark quenchers, black hole quenchers, ZEN quenchers, Dabcyl, BHQ quenchers, BBQ quenchers, Atto quenchers, TAMRA, and MGB.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### Image Processing

In various embodiments, images are processed prior to analysis. Image processing can be performed using any suitable method known in the art. Additional details on image processing for fluorescence microscopy are provided in Sanderson et al., Cold Spring Harb Protoc. 2014 October; 2014 (10); Baggett D W, et al. Front Bioinform. 2022 Jun. 6; 2:897238; Hallou A, et al. Development. 2021 Sep. 15; 148 (18): dev199616; and Uchida S. Dev Growth Differ. 2013 May; 55 (4): 523-49. In some embodiments, the images are processed to reduce or remove background noise. In some embodiments, the background noise is reduced or removed via application of a Laplacian of Gaussian filter. In some embodiments, a different Laplacian of Gaussian filter is applied to each image to achieve an approximately uniform background noise level between images. In some embodiments, further filters are applied to remove anomalous or artifactual signal. In some embodiments, the anomalous or artifactual signal is a small spot and/or a dim spot. In some embodiments, anomalous or artifactual signal is identified for removal by matching spots present at similar intensities in multiple channels.

In some embodiments, signal (e.g., one or more spots) present in the image after background subtraction are each mapped to a cell. In some embodiments, mapped signal is reconstructed using cell identification and a join algorithm. In some embodiments, the join algorithm is a fuzzy join.

In some embodiments, loci representing T7-amplified signals are detected manually. In some embodiments, loci representing T7-amplified signals are detected through application of a minimum size threshold and/or co-localization to the nucleus. In some embodiments, loci representing T7-amplified signals are detected through deep learning with manually labelled training data. Additional details on applications of deep learning for image classification are provided in Pachitariu, M. & Stringer, C., Nat Methods 19, 1634-1641 (2022).

### Detection of Additional Analytes

In various embodiments, a method provided herein further comprises detection of additional analytes (i.e., analytes other than a nucleic acid sequence of interest). In some embodiments, the additional analytes comprise one or more of protein, RNA, DNA stained in a non-sequence specific manner, DNA with a specific sequence, DNA mutations, lipids, including but not limited to phospholipids and sphingolipids, carbohydrates including but not limited to monosaccharides and polysaccharides, metabolites, small molecules, cellular structures, and tissue structures. Additional analytes can be detected by any suitable method known in the art.

In some embodiments, a method further comprises detecting the presence, absence, amount and/or localization of one or more protein analytes in the cells. In some embodiments, the one or more protein analytes are detected by immunofluorescence microscopy, direct immunofluorescence microscopy, indirect immunofluorescence microscopy, DNA-conjugated antibodies detectable by fluorescence (e.g., the CODEX system or Immuno-SABER), hybridization chain reaction (HCR) immunofluorescence microscopy, mass cytometry, aptamer-based detection of proteins, and multiplexed immunofluorescence based on cleavable fluorescent dyes (e.g., a chemically cleavable dye or photo-cleavable dye), InSituPlex Staining Method. In some embodiments, the one or more protein analytes are detected by immunofluorescence microscopy.

In some embodiments, the method further comprises detecting the presence, absence, amount and/or localization of one or more RNA analytes in the cells. In some embodiments, the one or more RNA analytes are detected by hybridization chain reaction (HCR), Fluorescence in situ hybridization (FISH), single-molecule fluorescence in situ hybridization (smFISH), RNAscope, transcriptome-wide or partially transcriptome-wide RNA in situ hybridization-based methods (e.g., MERFISH, seqFISH, or Digital Spatial Profiling), transcriptome-wide or partially transcriptome-wide RNA in situ sequencing methods (e.g., BAR-seq, BOLORAMIS, FISSEQ, Expansion-Seq (ExSeq), orSTARmap), and transcriptome-wide or partially transcriptome-wide RNA sequencing methods that preserve some spatial information (e.g., 10× Genomics Visium). In some embodiments, the one or more RNA analytes are detected by hybridization chain reaction (HCR).

### II. Engineered Cells

The present disclosure also provides for engineered cells or a population of engineered cells produced using a method disclosed herein. In some embodiments, the engineered cell or population of engineered cells comprises one or more exogenous promoters integrated into the genome of the cell. In some embodiments, the population of engineered cells comprises an exogenous promoter juxtaposed to exogenous nucleic acid sequences. In some embodiments, the population of engineered cells comprises an exogenous promoter juxtaposed to endogenous genomic regions comprising genomic sequences that are variable between cells within the population. In some embodiments, the exogenous promoter is capable of driving expression of the genomic sequences that are variable between cells within the population. In some embodiments, the one or more exogenous promoters are integrated in a site-specific manner. In some embodiments, the one or more exogenous promoters are incorporated at a random site in the genome of the one or more mammalian cells. In some embodiments, the one or more exogenous promoters are integrated into the genome of the cell by site-specific nucleases. In some embodiments, the one or more exogenous promoters are integrated into the genome of the cell by site-specific recombinases. In some embodiment, the population of engineered cells comprises an exogenous promoter juxtaposed to exogenous nucleic acids that are stably retained in the nucleus of the cell but do not integrate into the genome. In some embodiments, the population of engineered cells comprises an exogenous promoter juxtaposed to exogenous nucleic acids that are part of a plasmid or an artificial chromosome.

In some embodiments, the exogenous promoter and the genomic sequence that is variable between cells within the population are separated by no more than 2 kilobases (kb), no more than 1.5 kb, no more than 1 kb, no more than 900 (base pairs) bp, no more than 800 bp, no more than 700 bp, no more than 600 bp, no more than 500 bp, no more than 400 bp, no more than 300 bp, no more than 250 bp, no more than 200 bp, no more than 150 bp, no more than 100 bp, no more than 50 bp, or 0 bp. In some embodiments, the exogenous promoter and the genomic sequence that is variable between cells within the population are separated by no more than 2 kb. In some embodiments, the exogenous promoter and the genomic sequence that is variable between cells within the population are separated by no more than 1.5 kb. In some embodiments, the exogenous promoter and the genomic sequence that is variable between cells within the population are separated by no more than 1 kb. In some embodiments, the exogenous promoter and the genomic sequence that is variable between cells within the population are separated by no more than 900 bp. In some embodiments, the exogenous promoter and the genomic sequence that is variable between cells within the population are separated by no more than 800 bp. In some embodiments, the exogenous promoter and the genomic sequence that is variable between cells within the population are separated by no more than 700 bp. In some embodiments, the exogenous promoter and the genomic sequence that is variable between cells within the population are separated by no more than 600 bp. In some embodiments, the exogenous promoter and the genomic sequence that is variable between cells within the population are separated by no more than 500 bp. In some embodiments, the exogenous promoter and the genomic sequence that is variable between cells within the population are separated by no more than 400 bp. In some embodiments, the exogenous promoter and the genomic sequence that is variable between cells within the population are separated by no more than 300 bp. In some embodiments, the exogenous promoter and the genomic sequence that is variable between cells within the population are separated by no more than 250 bp. In some embodiments, the exogenous promoter and the genomic sequence that is variable between cells within the population are separated by no more than 200 bp. In some embodiments, the exogenous promoter and the genomic sequence that is variable between cells within the population are separated by no more than 150 bp. In some embodiments, the exogenous promoter and the genomic sequence that is variable between cells within the population are separated by no more than 100 bp. In some embodiments, the exogenous promoter and the genomic sequence that is variable between cells within the population are separated by no more than 50 bp. In some embodiments, the exogenous promoter and the genomic sequence that is variable between cells within the population are separated by 0 bp. In some embodiments, the separation is in a genomic DNA sequence, an exonic coding sequence, or an RNA sequence of the cell. In some embodiments, the separation is in the genomic of the cell. In some embodiments, the separation is in the exonic coding sequence of the cell. In some embodiments, the separation is in an RNA sequence of the cell comprising the exogenous promoter and the genomic sequence that is variable between cells within the population.

In some embodiments, more than one distinct exogenous promoters are integrated into the genome of the cell. In some embodiments, each exogenous promoter is incorporated at a specific site in the genome of the cell. In some embodiments, some exogenous promoters are incorporated at specific sites in the genome of the cell and other exogenous promoters are incorporated at random sites in the genome of the cell.

In some embodiments, the exogenous promoter is a sequence-specific RNA polymerase promoter. In some embodiments, the exogenous promoter is a phage promoter. In some embodiments, the exogenous promoter is selected from the group consisting of: a T7 promoter, a T3 promoter; and an SP6 promoter. In some embodiments, the exogenous promoter is a T7 promoter. In some embodiments, the exogenous promoter is a T3 promoter. In some embodiments, the exogenous promoter is an SP6 promoter.

In some embodiments, the engineered cells comprise one or more mammalian cells selected from stem cells, mesodermal cells, endodermal cells, ectodermal cells, cardiomyocytes, immune cells, epithelial cells, pneumocytes, club cells, paneth cells, pancreatic cells, stomach cells, goblet cells, gland cells, duct cells, centroacinar cells, brush border cells, endocrine cells, thyroid gland cells, pancreatic islet cells, mucous cells, pituitary cells, neurons, sensory neurons, receptor neurons, neuronal progenitors, cone cells, rod cells, interneurons, astrocytes, oligodendrocytes, ependymal cells, pituicytes, adipocytes, lipocytes, cells of the kidney or urinary system, reproductive cells, endothelial cells, extracellular matrix cells, contractile cells, skeletal muscle cells, cardiac muscle cells, blood cells, germ cells, nurse cells, or interstitial cells. In various embodiments, the engineered cells comprise one or more immune cells. In some embodiments, the immune cells are T cells, NK cells, B cells, macrophages, dendritic cells, mast cells, monocytes, neutrophils, basophils, eosinophils, hematopoietic stem cells, or immortalized immune cells. In some embodiments, the immune cells are T cells, NK cells, or B cells. In some embodiments, the immune cells express one or more cell therapy constructs (e.g., an engineered immune receptor). In some embodiments the immune cells express a chimeric antigen receptor (CAR). In some embodiments, the immune cells are chimeric-antigen receptor-expressing T (CAR-T) cells or chimeric-antigen receptor-expressing NK (CAR-NK) cells. In some embodiments, the immune cells are chimeric-antigen receptor-expressing T (CAR-T) cells. In some embodiments, the immune cells are chimeric-antigen receptor-expressing NK (CAR-NK) cells. In some embodiments, the biological sample comprises embryonic stem cells (ESCs) or induced pluripotent stem cells (iPSCs) or cells derived from ESCs or iPSCs, and optionally differentiated to a specific lineage.

In some embodiments, the population of engineered cells is homogenous. In some embodiments, the population of engineered cells is heterogeneous.

In some embodiments, the engineered cells comprise one or more cells consisting of cells from a single species. In some embodiments, the engineered cells comprise one or more cells from multiple species. In some embodiments, the engineered cells comprise human cells and mouse cells. In some embodiments, the engineered cells comprise human immune cells and mouse cells.

### III. Kits

The present disclosure also provides for kits for performing a method disclosed herein or for preparing an engineered cell or population of engineered cells. In some embodiments, the kit comprises (a) a sequence-specific RNA polymerase; (b) a reverse transcriptase enzyme; (c) reagents for in situ sequencing; and (d) instructions for using components (a)-(c) to determine the presence, absence, amount, and/or localization of a nucleic acid sequence of interest in one or more fixed mammalian cells in a biological sample in situ.

In some embodiments, the sequence-specific RNA polymerase is a phage RNA polymerase. In some embodiments, the sequence-specific RNA polymerase is selected from the group consisting of: a T7 RNA polymerase, a T3 RNA polymerase; and an SP6 RNA polymerase. In some embodiments, the sequence-specific RNA polymerase is a T7 promoter. In some embodiments, the RNA polymerase is a T3 RNA polymerase. In some embodiments, the sequence-specific RNA polymerase is an SP6 RNA polymerase.

In some embodiments, the reverse transcriptase is an AMV reverse transcriptase. In some embodiments, the reverse transcriptase is an MMLV reverse transcriptase. In some embodiments, the reverse transcriptase is an engineered reverse transcriptase.

In some embodiments, the in situ sequencing is sequencing by synthesis. In some embodiments, the reagents for sequencing by synthesis comprise (i) a plurality of detectably labeled nucleotides; and (ii) a DNA polymerase. In some embodiments, the detectably labeled nucleotides comprise a cleavable detectable label. In some embodiments, the detectably labeled nucleotides comprise a reversible chain terminator modification.

In some embodiments, the in situ sequencing is sequencing-by-ligation. In some embodiments, the reagents for sequencing by ligation comprise (i) a plurality of detectably labeled oligonucleotides containing degenerate bases; and (ii) a DNA ligase. In some embodiments, the detectably labeled oligonucleotides comprise a cleavable detectable label. In some embodiments, the detectably labeled oligonucleotides comprise a reversible chain terminator modification.

In some embodiments, the in situ sequencing is sequencing-by-avidity. In some embodiments, the reagents for sequencing-by-avidity comprise (i) a plurality of detectably labeled avidites; and (ii) an engineered DNA polymerase. In some embodiments, the detectably labeled avidites comprise a cleavable detectable label.

In some embodiments, the kit further comprises: (i) a DNA primer comprising: (A) a 3′ nucleic acid sequence that is complementary to a portion of a target RNA flanking the nucleic acid sequence of interest, and (B) a 5′ nucleic acid sequence comprising a sequence-specific RNA polymerase promoter; or further instructions for designing said DNA primer; and (ii) further instructions for performing reverse transcription on the target RNA using the DNA primer to generate a cDNA molecule. In some embodiments, the 5′ nucleic acid sequence of the DNA primer comprising the sequence-specific RNA polymerase promoter is dsDNA. In some embodiments, the dsDNA is hybridized dsDNA or a hairpin.

In some embodiments, the kit further comprises: (i) reagents for performing second strand synthesis on the cDNA molecule; and/or (ii) further instructions for performing said second strand synthesis.

In some embodiments, the methods provided for herein do not comprise a DNA degradation step. For example, in some embodiments, the methods do not comprise reacting a sample (e.g., any sample or reaction mixture produced during the performance of the method) with a DNase.

### EXAMPLES

The following Examples are merely illustrative and are not intended to limit the scope or content of the invention in any way.

### Example 1: Parameters for Amplification of Variable Barcode Sequences

### Constructs & Cell Line Engineering

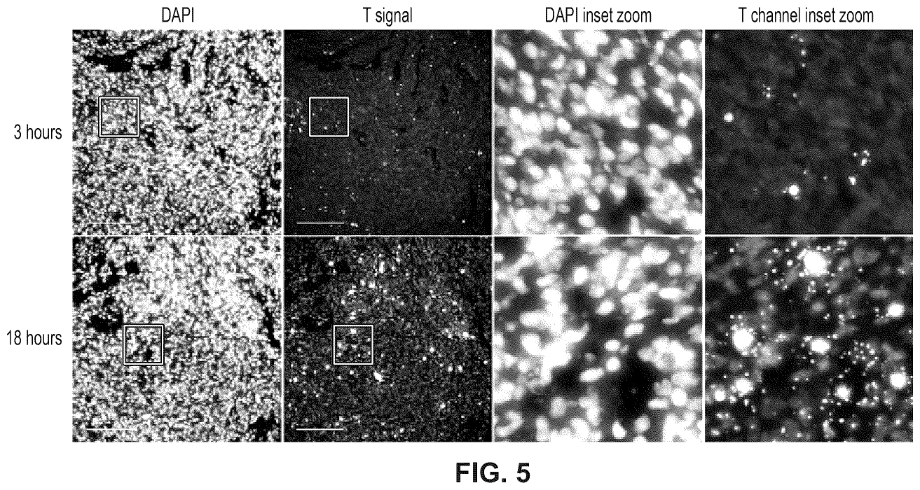

A construct was designed to contain a T7 phage promoter upstream of a nucleic acid region of interest. For cell lines, the DNA constructs were introduced into the target cells by lentiviral transduction. Lentivirus was added to mammalian cells to achieve desired multiplicity of infection, which resulted in the construct integrating into the genome of the mammalian cells. The tissue samples shown in FIGS. 4, 5, 6, and 7 are from mice with human patient-derived xenograft (PDX) tumors, and the mice have also been injected with human CAR T cells. Some of the CAR T cells have the T7 promoter (e.g., T7) barcode construct integrated into the genome. The CAR T cells had infiltrated the PDX tumor at the time of tissue preparation. Briefly, the tissue samples were prepared by standard FFPE preparation techniques, including fixation with formaldehyde and embedding in paraffin. The paraffin-embedded FFPE blocks were cut into 5 μm thick sections which were mounted on glass slides. To perform the experiments described, the tissue samples were first deparaffinized, rehydrated, and underwent antigen retrieval.

RNA Transcription from Phage Promoter in Fixed Cells or Tissue

Cells were washed in PBS, fixed, and permeabilized with triton detergent. T7 reaction mix (buffer, NTPs, T7 polymerase, and RNase inhibitor) was added to cells. The T7 reaction was incubated at a constant uniform temperature (37° C.) for the specific reaction time (6 h) to generate RNA from DNA using the phage promoter. Control groups for cells and tissue samples were also prepared with no T7 reaction performed. After reaction incubation, cells were washed and newly transcribed RNA were fixed in place using formaldehyde.

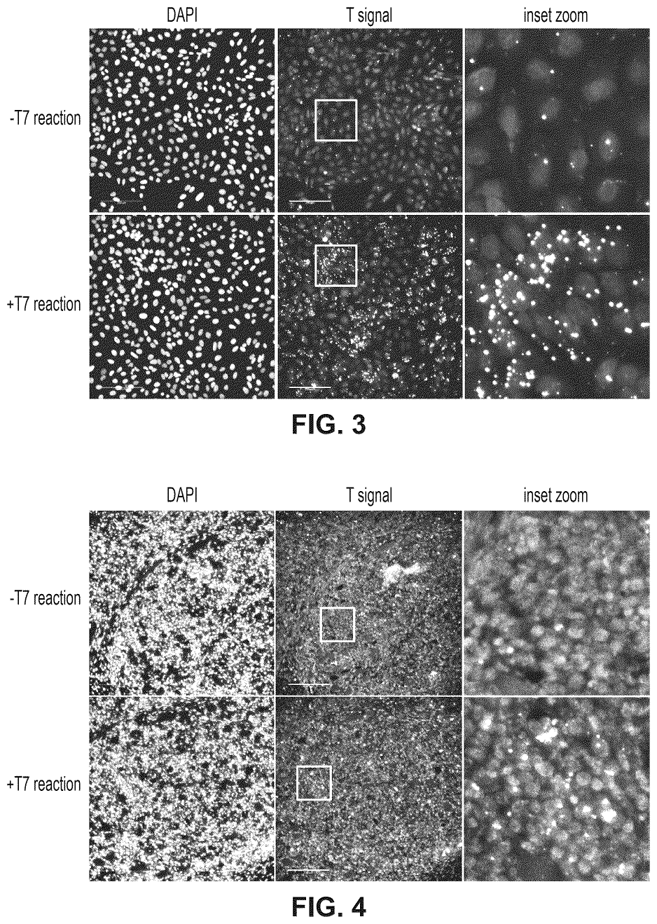

The sample then underwent reverse transcription to generate cDNA from RNA transcripts by hybridization of a primer and treatment with a reverse transcriptase enzyme. The cDNA was then fixed in place and used as a target for padlock binding, gap-fill, and ligation to generate a circular DNA template containing the sequence of interest. A primer was hybridized to the circular DNA and rolling circle amplification was performed on the circular template to generate many repeated single-stranded DNA (ssDNA) copies of the sequence of interest. Cells were washed and RCA amplicons were fixed. Sequencing-by-synthesis was performed to read out the sequence downstream of the T7 promoter by first adding a sequencing primer and then adding fluorescent nucleotides with reversible terminator sequences (to enable incorporation of only a single nucleotide per cycle). Incorporated nucleotides were read in situ by fluorescent microscopy. FIG. 3 shows cell samples and FIG. 4 shows tissue samples where the T7 reaction was performed (bottom panels) compared to control samples where no T7 reaction was performed (top panels). DAPI shows cell nuclei, whereas “T signal” shows detected nucleic acids. These results indicate that in situ transcription followed by fluorescence in situ sequencing can be used to detect target nucleic acid sequences in fixed cell samples and FFPE samples.

In another experiment, tissue samples were prepared as above. Samples were washed in PBS, fixed, and permeabilized with triton detergent. T7 reaction mix (buffer, NTPs, T7 polymerase, and RNase inhibitor) was added to cells. The T7 reaction was incubated at for reaction times of 3 hours or 18 hours to generate RNA from DNA using the phage promoter. Reverse transcription, RCA, and sequencing by synthesis were carried out as above. FIG. 5 shows images of tissue samples where the T7 reaction was incubated for 3 hours (top panels) or 18 hours (bottom panels). These results indicate that increased T7 reaction duration increases the signal of detected barcode nucleic acids.

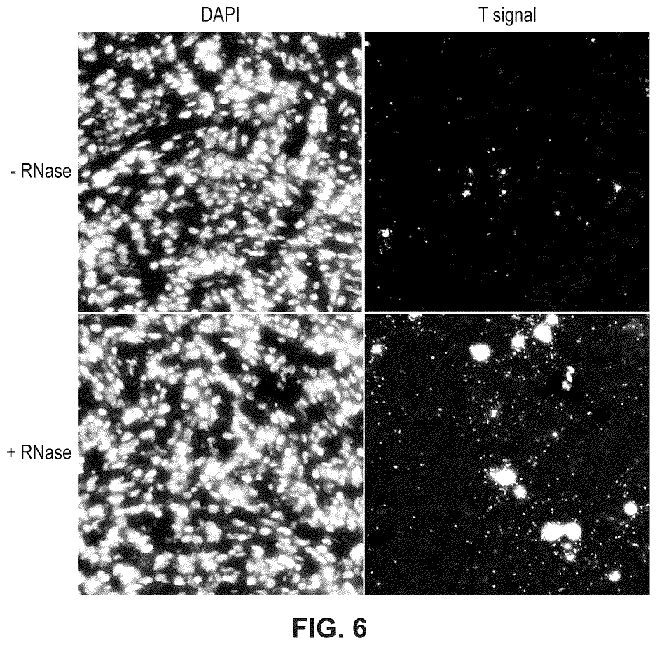

In another experiment, tissue samples were prepared as above. Prior to the T7 reaction, cells were treated with RNase to degrade endogenous RNAs. The T7 reaction, reverse transcription, RCA, and sequencing-by-synthesis were performed as above. FIG. 6 shows images of tissue samples that were treated with RNase (bottom panels) or control (top panels). These results indicate that an RNase treatment prior to T7 amplification can improve specificity of detection without loss of barcode signal, which can be advantageous.

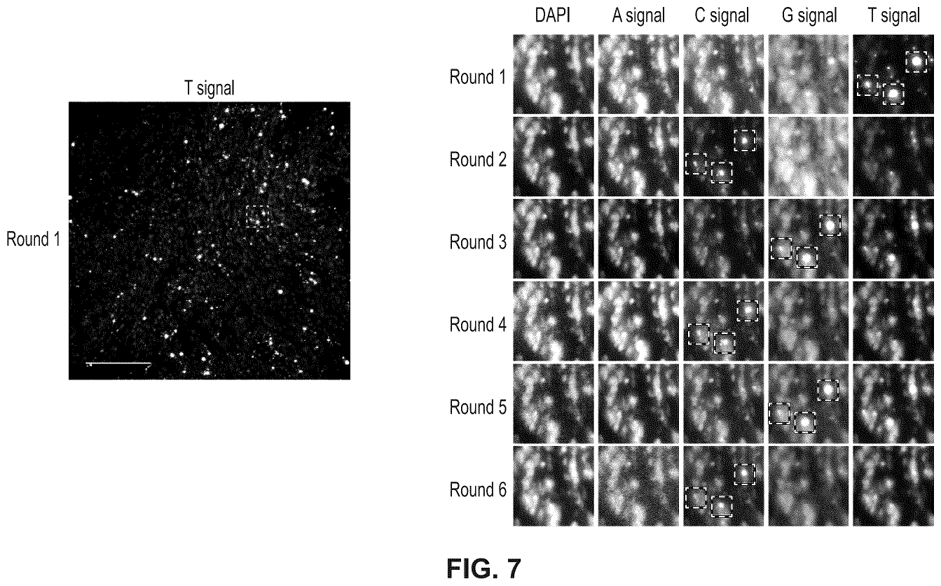

In another experiment, tissue samples were prepared and the T7 reaction, reverse transcription, and RCA were all performed as above. Six successive rounds of sequencing-by-synthesis and imaging were then performed on the samples. FIG. 7 shows images of DAPI (nuclei), adenine, cytosine, guanine, and thymine channels in tissue samples following each successive round of sequencing by synthesis. The results show that the method yields specific signal for each round of sequencing-by-synthesis within minimal cross-channel detection.

### Example 2: Improved Detection of Nucleic Acid Sequences by Amplification of Variable Barcode Sequence Using an Integrated Promoter within a Biological Sample, Followed by Amplification of cDNA Product

Tissue samples or cell samples are prepared as in Example 1 above with one group expressing a construct that does not include the T7 promoter system. The T7 reaction, reverse transcription, RCA, and sequencing-by-synthesis are performed as in Example 1 above. Images of in situ sequencing results from samples containing the T7 promoter system are compared to images from samples that do not contain the T7 promoter system.

### Example 3: Amplification of Variable Barcode Sequence Using an Integrated Promoter within a Biological Sample, Followed by Amplification of cDNA Product

### Constructs & Cell Line Engineering

For cell lines, constructs are designed to contain the phage promoter of interest (T3, T7, SP6, etc.) upstream of a nucleic acid region of interest (e.g., a barcode or other variable nucleic acid sequence). The region of interest can be an endogenous sequence or an exogenously introduced sequence. A phage transcription termination sequence is optionally included downstream. In some embodiments, a nucleic spacer is included between the promoter and nucleic acid region.

Constructs containing promoter and barcode to be tracked are genetically introduced into mammalian cells. The DNA constructs are introduced into the cell by lentivirus. Lentivirus is first produced by incubating the transfer plasmid with packaging and envelope plasmids and a transfection reagent and adding to 293T cells. Lentiviral supernatant is harvested from the 293T cells 48 or 72 hours later following removal of any contaminating cells and either stored at −80° C. or immediately used. Lentivirus is added to mammalian cells at specified titers to achieve desired multiplicity of infection, which results in the construct integrating into the genome of these mammalian cells. Following several days incubation and cell expansion, the percentage of cells expressing the construct is determined through flow cytometry, drug resistance, or other methods. The cells are then expanded, passaged, frozen or used for downstream assays. The cells can also be engineered to contain construct using other viral vectors including retroviral or AAV or other methods such as gene editing.

RNA Transcription from Phage Promoter in Fixed Cells or Tissue

For fixed cells, cells are washed in PBS, fixed, and permeabilized with triton detergent. T7 reaction mix is added to cells (T7 reaction includes buffer, NTPs, T7 polymerase, and RNase inhibitor). The T7 reaction is incubated at a constant uniform temperature (37-42° C.) for the specific reaction time (3 h-18 h) to generate RNA from DNA using the phage promoter. After reaction incubation, cells are washed and newly transcribed RNA are fixed in place (e.g., using formaldehyde).

The sample then undergoes reverse transcription to generate DNA from RNA transcripts by hybridization of a primer and treatment with a reverse transcriptase enzyme. cDNA is then fixed in place (e.g., using formaldehyde or glutaraldehyde) and all or a portion of the complementary RNA is optionally digested. cDNA is then used as a target for padlock binding, gap-fill, and ligation to generate a circular DNA template containing the variable sequence. A primer is hybridized to the circular DNA and rolling circle amplification is performed on the circular template to generate many repeated ssDNA copies of the nucleic acid region of interest. Cells are washed and RCA amplicons are fixed. Sequencing-by-synthesis is performed to readout the barcode sequence downstream of the T7 promoter by first adding a sequencing primer and then adding fluorescent nucleotides with reversible terminator sequences (to enable incorporation of only a single nucleotide per cycle). Incorporated nucleotides are read in situ by fluorescent microscopy. Fluorophores and reversible terminators are cleaved following imaging in each round, and the sequencing by synthesis process repeated to get the barcode sequence of interest.

For tissues, the tissue samples that contain cells with the Promoter (e.g., T7) barcode constructs are first deparaffinized, rehydrated, and undergo antigen retrieval. Tissue then undergoes the workflow as above.

### Example 4: Amplification of Variable Barcode Sequence Using an Integrated Promoter within a Biological Sample without Amplification of cDNA Product

### Constructs & Cell Line Engineering

Constructs are designed to contain the phage promoter of interest (T3, T7, SP6, etc.) upstream of a nucleic acid region of interest (e.g., a barcode or other variable nucleic acid sequence). The region of interest can be an endogenous sequence or an exogenously introduced sequence. A phage transcription termination sequence is optionally included downstream. In some embodiments, a nucleic acid spacer is included between the promoter and nucleic acid region.

Constructs containing promoter and barcode to be tracked are genetically introduced into mammalian cells. The DNA constructs are introduced into the cell by lentivirus. Lentivirus is first produced by incubating the transfer plasmid with packaging and envelope plasmids and a transfection reagent and adding to 293T cells. Lentiviral supernatant is harvested from the 293T cells 48 or 72 hours later following removal of any contaminating cells and either stored at −80° C. or immediately used. Lentivirus is added to mammalian cells at specified titers to achieve desired multiplicity of infection, which results in the construct integrating into the genome of these mammalian cells. Following several days incubation and cell expansion, the percentage of cells expressing the construct is determined through flow cytometry, drug resistance, or other methods. The cells are then expanded, passaged, frozen or used for downstream assays. Cells can also be engineered to contain construct using other viral vectors including retroviral or AAV or other methods such as gene editing.

RNA Transcription from Phage Promoter in Fixed Cells or Tissue

For cell samples, cells are washed in PBS, fixed, and permeabilized with triton detergent. T7 reaction mix is added to cells (T7 reaction includes buffer, NTPs, T7 polymerase, and RNase inhibitor). The T7 reaction is incubated at a constant uniform temperature (37-42° C.) for the specific reaction time (3h-18h) to generate RNA from DNA using the phage promoter. After reaction incubation, cells are washed and newly transcribed RNA are fixed in place (e.g., using formaldehyde).

The sample then undergoes reverse transcription to generate DNA from RNA transcripts by hybridization of a primer and treatment with a reverse transcriptase enzyme. The primer contains one region with complementarity to the RNA (3′ end), and another region (5′ end) containing a T7 promoter sequence. Following reverse transcription, cDNA is optionally fixed in place (e.g., using formaldehyde or glutaraldehyde) and all or a portion of the complementary RNA is optionally digested. Sequencing-by-synthesis is directly performed to readout the barcode sequence from the cDNA product by first adding a sequencing primer and then adding fluorescent nucleotides with reversible terminator sequences (to enable incorporation of only a single nucleotide per cycle). Incorporated nucleotides are read in situ by fluorescent microscopy. Fluorophores and reversible terminators are cleaved following imaging in each round, and the sequencing by synthesis process repeated to get the barcode sequence of interest.

For tissue samples, the tissue samples that contain cells with the Promoter (e.g., T7) barcode constructs are first deparaffinized, rehydrated, and undergo antigen retrieval. Tissue then undergoes the workflow as above.

### Example 5: Amplification and Sequencing of Endogenous Sequence or Exogenous Sequence without Integrated Promoter—Second Strand Synthesis Version

Cells or tissues containing RNAs of interest (endogenous or exogenously introduced) are fixed or processed as described above.

For fixed cells—cells are washed in PBS, fixed, and permeabilized with triton detergent. The sample undergoes reverse transcription to generate DNA from endogenous RNA transcripts by hybridization of a primer and treatment with a reverse transcriptase enzyme. The primer contains one region with complementarity to the RNA (3′ end), and another region (5′ end) containing a T7 promoter sequence. Following reverse transcription, cDNA is optionally fixed in place (e.g., using formaldehyde or glutaraldehyde) and all or a portion of the RNA is optionally digested. Second strand synthesis is performed using a primer that hybridizes to the cDNA such that at the end of second strand synthesis, the T7 promoter and nucleic acid region of interest are both double-stranded, with T7 promoter driving transcription of the region of interest.

T7 reaction mix is added to cells (T7 reaction includes buffer, NTPs, T7 polymerase, and RNase inhibitor). The T7 reaction is incubated at a constant uniform temperature (37-42° C.) for the specific reaction time (3h-18h) to generate RNA from DNA using the phage promoter. After reaction incubation, cells are washed and newly transcribed RNA are fixed in place (e.g., using formaldehyde). A second RT reaction is performed on these RNA transcripts following the examples outlined above, and in situ sequencing is performed using rolling circle amplification (e.g., following example 1 above) or directly (e.g., following example 2 above).

Amplification and Sequencing of Endogenous Sequence or Exogenous Sequence without Integrated Promoter—Double-Stranded Primer

Cells or tissues containing RNAs of interest (endogenous or exogenously introduced) are fixed or processed as described above.

For cell samples, cells are washed in PBS, fixed, and permeabilized with triton detergent. The sample undergoes reverse transcription to generate DNA from endogenous RNA transcripts by hybridization of a primer and treatment with a reverse transcriptase enzyme. The primer contains one region with complementarity to the RNA (3′ end), and another region (5′ end) containing a double-stranded T7 promoter sequence (e.g., a hairpin from a single oligo, two short oligos bound to each other, or other configurations to make part of the T7 promoter region double-stranded). Following reverse transcription, cDNA is optionally fixed in place (e.g., using formaldehyde or glutaraldehyde) and RNA is optionally digested. T7 reaction mix is added to cells (T7 reaction includes buffer, NTPs, T7 polymerase, and RNase inhibitor). The T7 reaction is incubated at a constant uniform temperature (37-42° C.) for the specific reaction time (3h-18h) to generate RNA from DNA using the phage promoter. After reaction incubation, cells are washed and newly transcribed RNA are fixed in place (e.g., using formaldehyde). A second RT reaction is performed on these RNA transcripts following the examples outlined above, and in situ sequencing is performed using rolling circle amplification (e.g., following example 2 above) or directly in the prior steps (e.g., following example 3 above).

The methods provided for herein provide surprising and unexpected results as compared to previous methods. For example U.S. Pat. No. 11,421,273 and Aksary et al. Nat Biotechnol 38, 66-75 (2020), termed “Zombie” (Zombie is Optical Measurement of Barcodes by In situ expression.” provide methods for in situ readout of DNA barcodes via in vitro transcription, but such methods are limited, for example, because such methods can only detect barcodes of at least 20 nucleotides in length, which is in contrast to the methods provided for herein. Zombie is also deficient because it requires treatment with DNase, which degrades DNA in the sample, preventing further probing of a target DNA and it cannot be applied to formalin-fixed samples. Thus, the embodiments and examples provided for herein overcome these issues as well as other issues that other methods have.

### Example 6: Amplification of Variable Barcode Sequence Using an Integrated Promoter within a Biological Sample, Followed by Amplification of cDNA Product

### Constructs & Cell Line Engineering

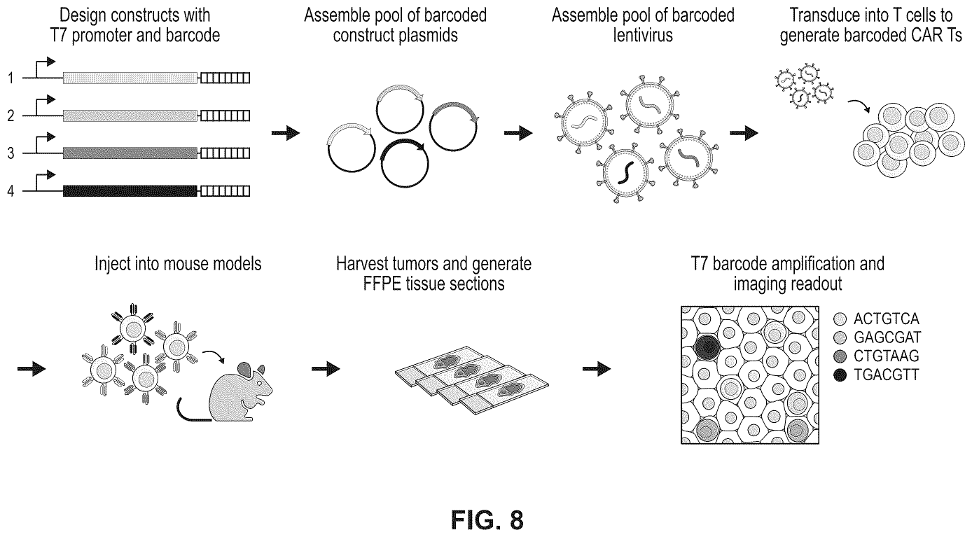

This example workflow is depicted in FIG. 8. Constructs were designed to contain the promoter of interest (e.g., T3, T7, SP6, etc.) upstream of a nucleic acid sequence or region of interest (e.g., a barcode or other variable nucleic acid sequence). The sequence of interest was an endogenous sequence or an exogenously introduced sequence. An optional phage transcription termination sequence was included downstream. In some instances, a nucleic acid spacer was included between the promoter and nucleic acid sequence of interest.

DNA constructs containing the phage promoter and barcode to be tracked were genetically introduced into mammalian cells by lentiviral transduction. Lentivirus was produced by incubating the construct-containing transfer plasmid with packaging and envelope plasmids and a transfection reagent and then adding this transfection mixture to 293T cells. Lentiviral supernatant was harvested from the 293T cells 48 or 72 hours later following removal of any contaminating cells. Lentivirus was then either stored at −80° C. or immediately used. Lentivirus was added to mammalian cells at specified titers to achieve the desired multiplicity of infection, which resulted in the construct integrating into the genome of these mammalian cells. In some examples, the mammalian cells transduced with the lentivirus were human primary T cells. In some examples, lentiviral transduction resulted in the T cells expressing one or more Chimeric Antigen Receptors (CARs), endogenous human proteins, mutant or truncated versions of human proteins, synthetic proteins, proteins originating from other organisms, non-coding RNAs such as short-hairpin RNAs (shRNAs), or combinations of one or more of these classes of proteins or RNAs. Following several days incubation and cell expansion, the percentage of cells expressing the construct was determined through flow cytometry, drug resistance, or other methods. The cells were then expanded, passaged, frozen, or used for downstream assays. Construct containing cells could also be engineered using other viral vectors including retroviral or AAV or engineered using other methods such as gene editing.

### Mouse Models and Tissue Sample Preparation

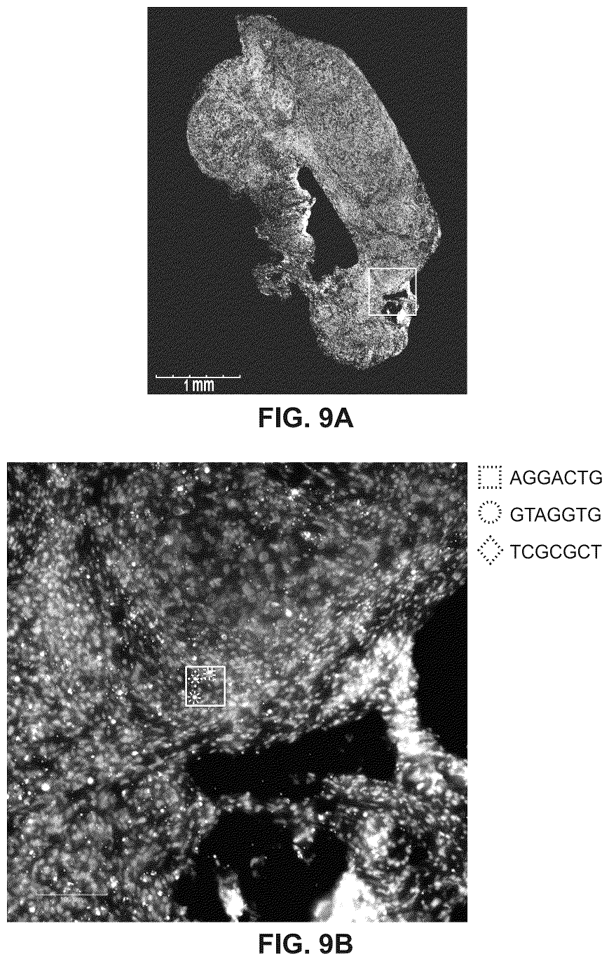

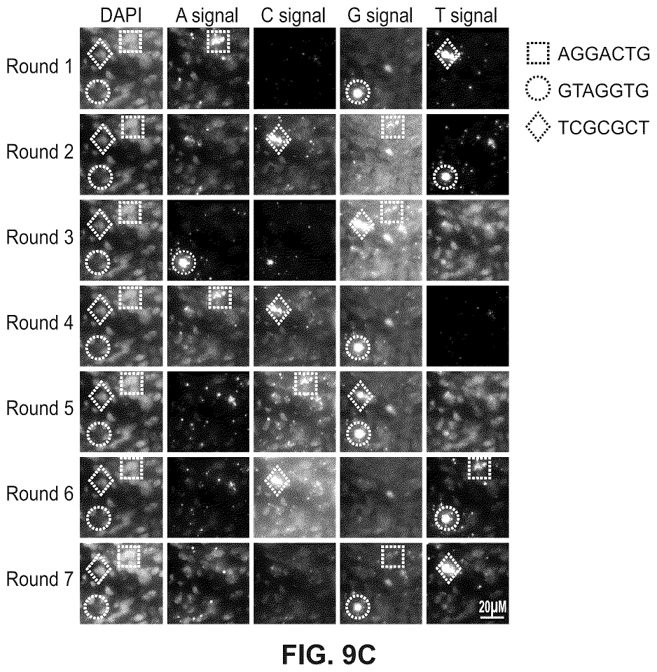

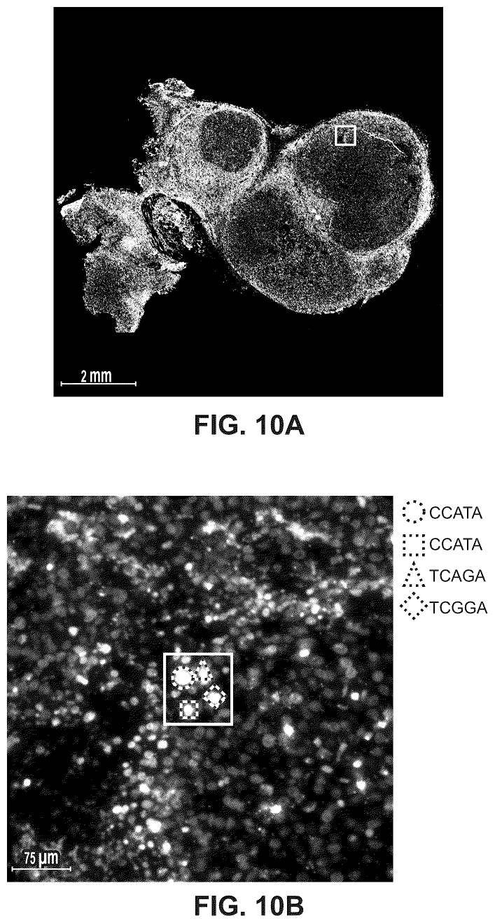

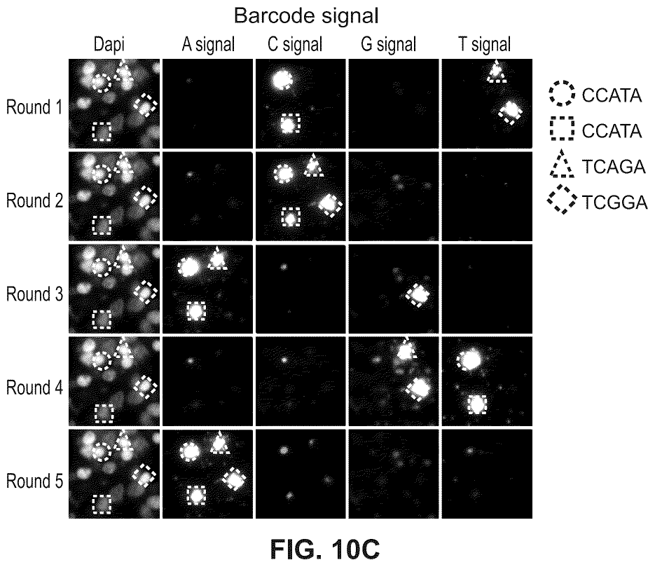

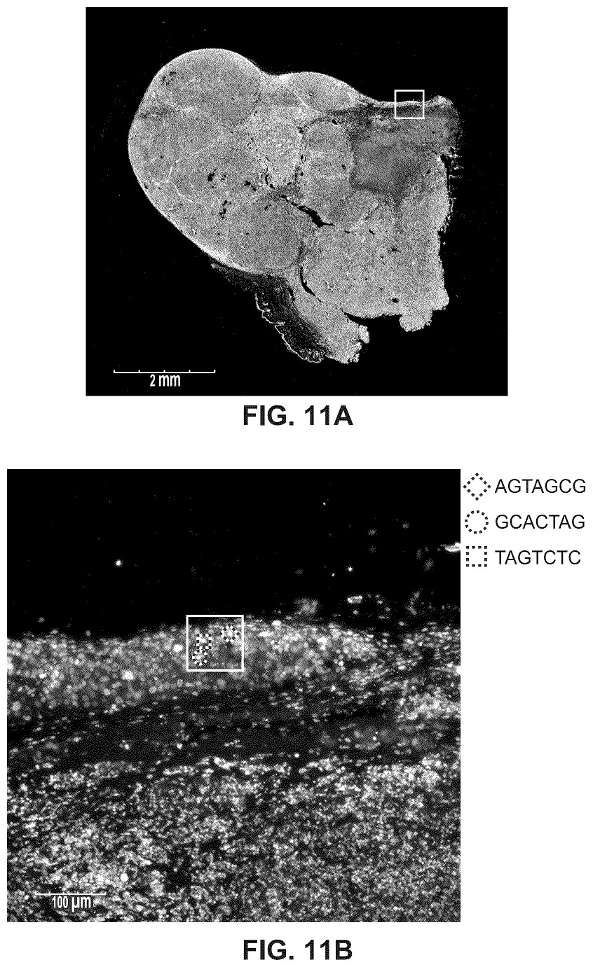

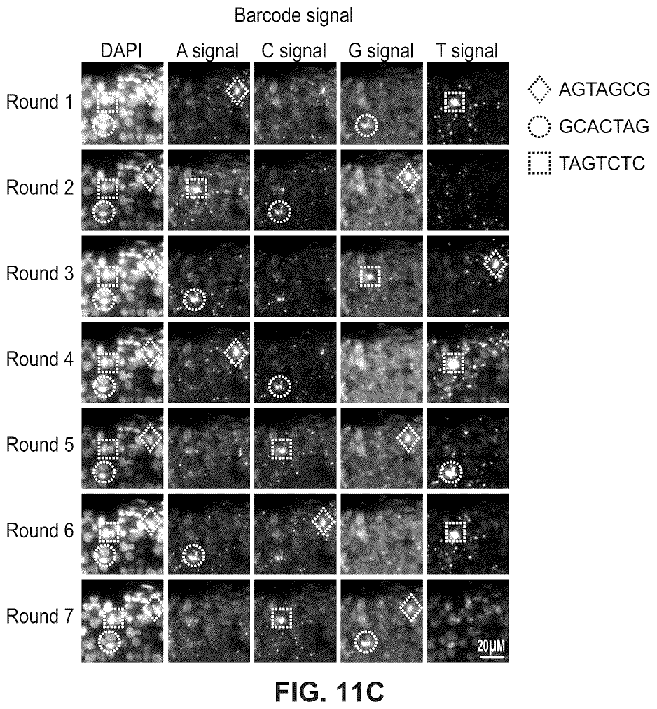

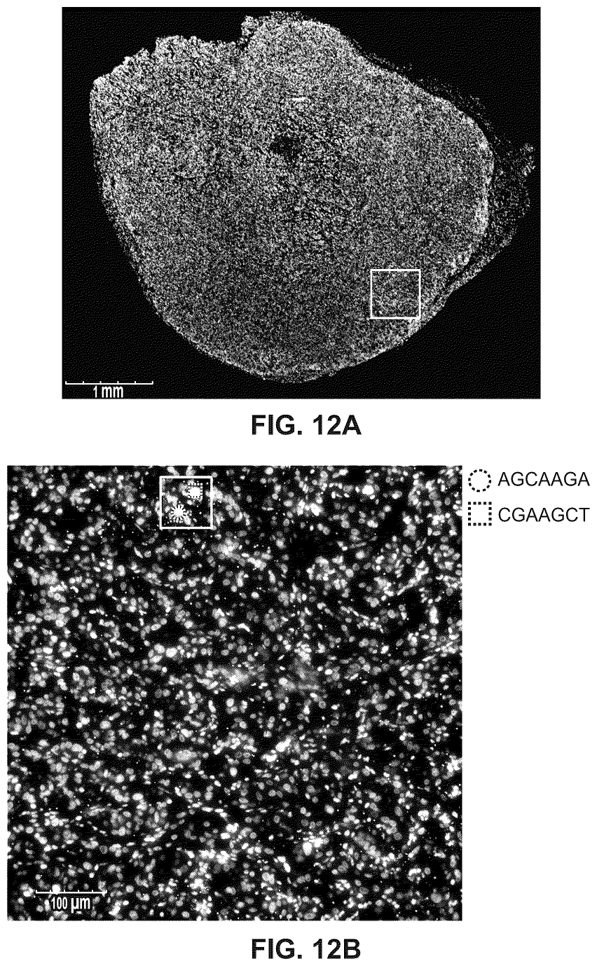

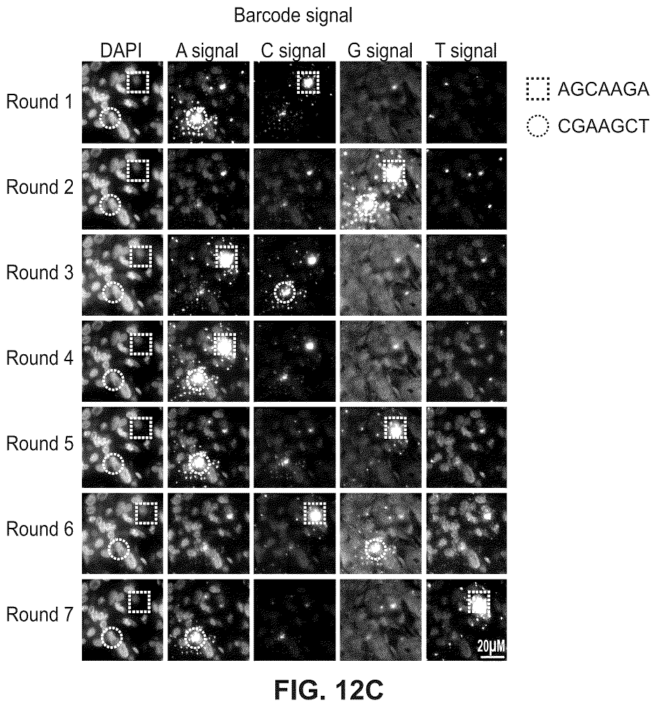

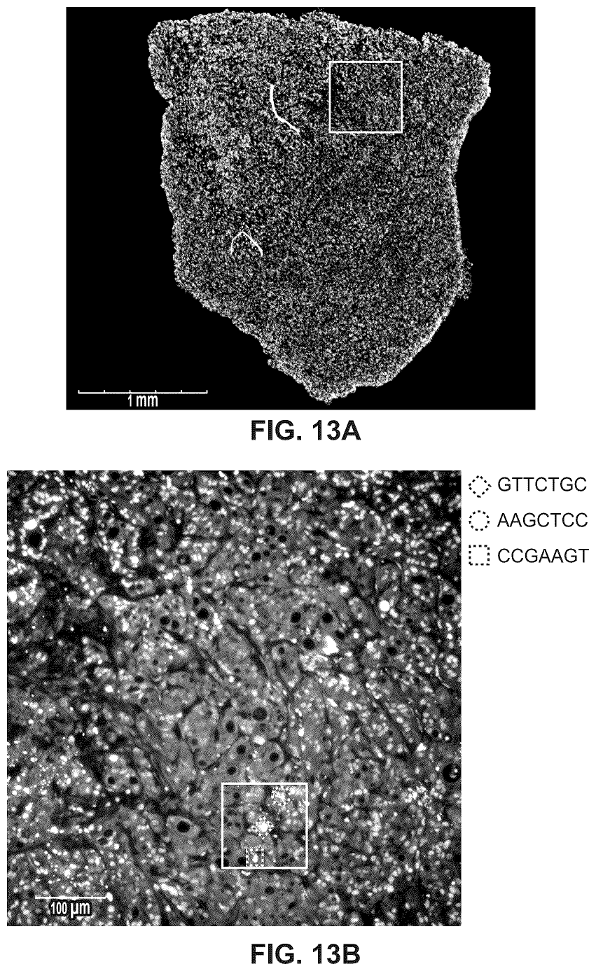

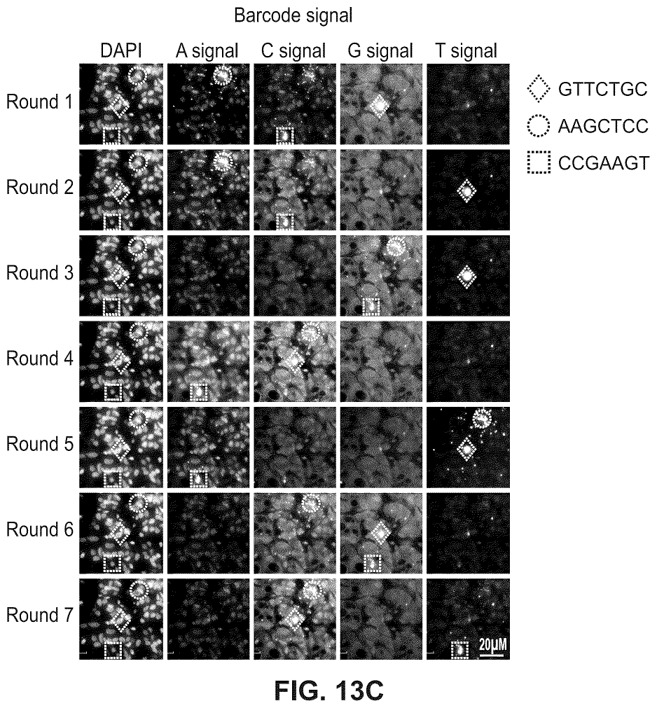

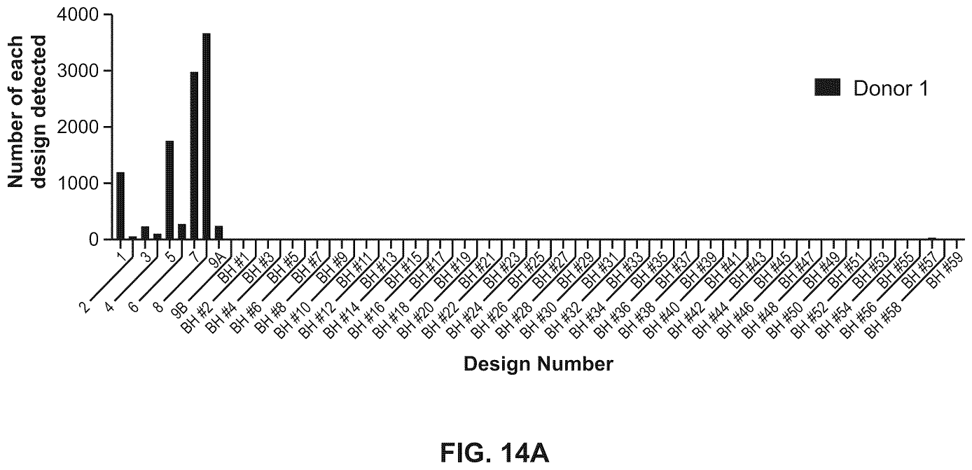

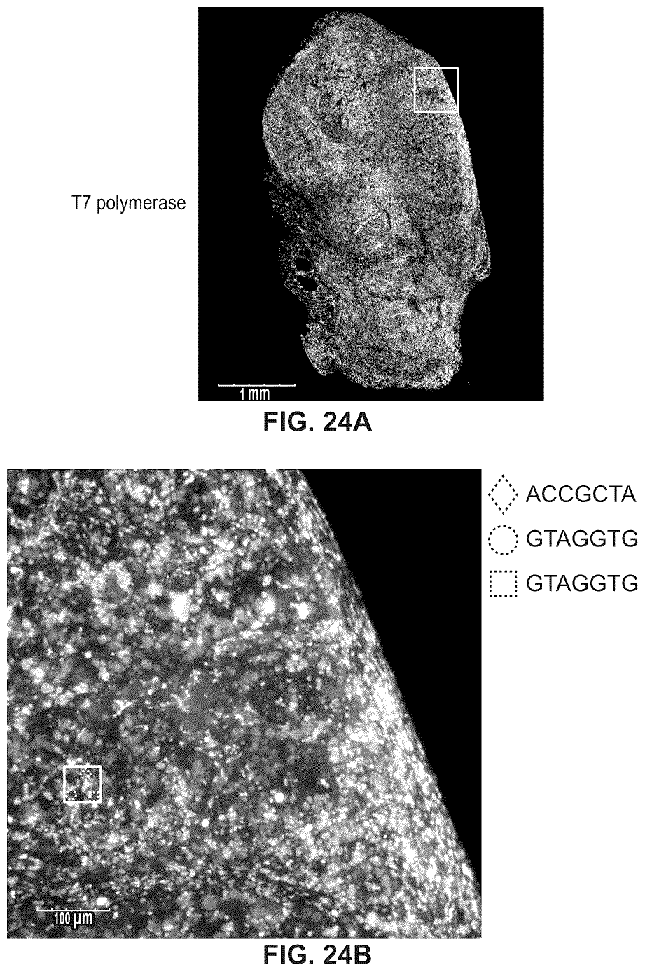

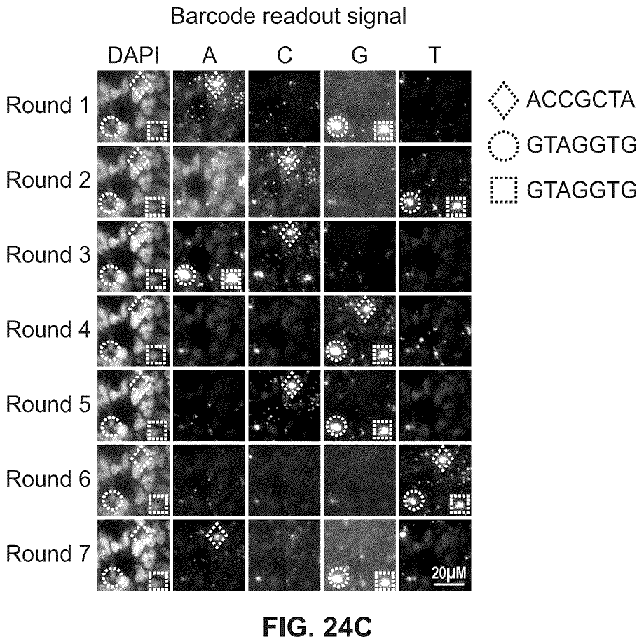

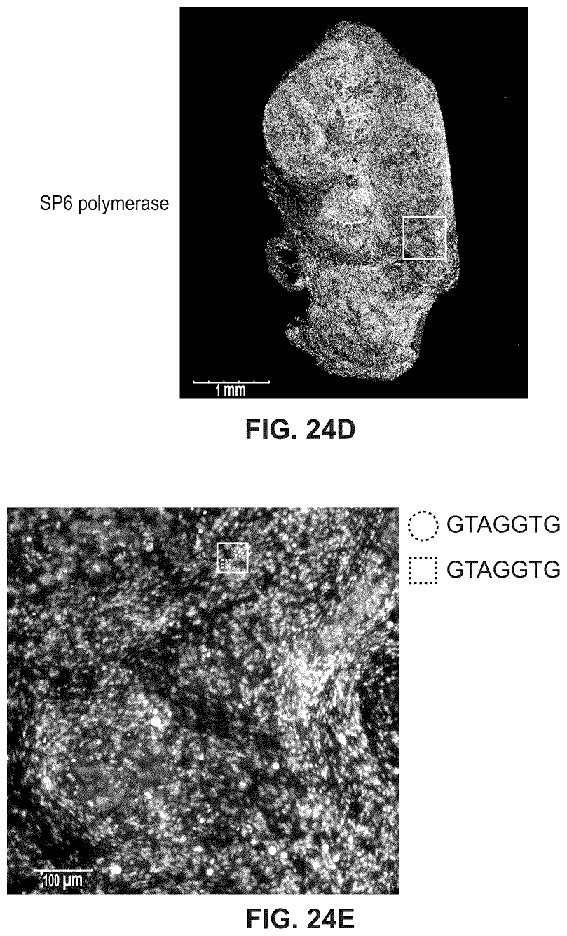

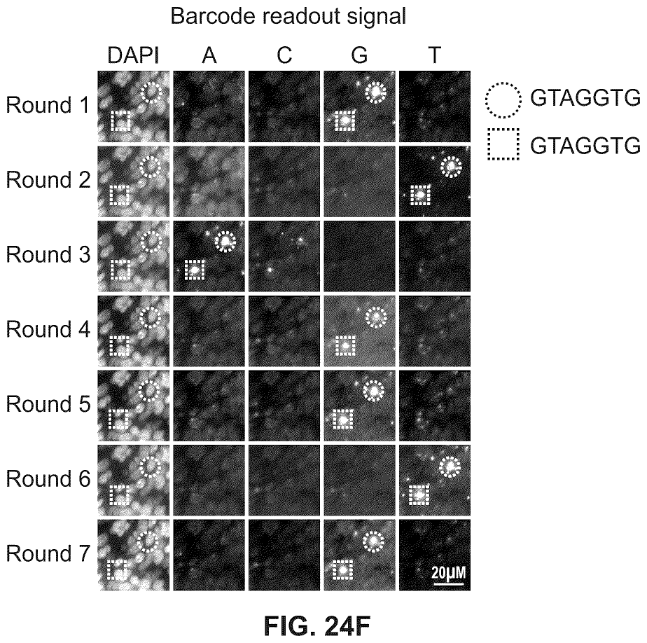

Cells genetically modified to contain the construct containing the phage promoter and barcode were prepared for injection into mouse cancer models by washing the cells with PBS and resuspending the cells to the desired concentration for injection. Between 1×10⁶-10×10⁶ human T cells were injected into immunocompromised NOD-scid gamma (NSG) mice pre-implanted or pre-injected with human patient-derived xenograft (PDX) tumors or human cancer cell line-derived xenograft (CDX) tumors. The tissue samples shown in FIGS. 9A-9C and 24A-24F were from mice with orthotopic gastric PDX tumors, the tissue samples shown in FIGS. 10A-10C and 11A-11C were from mice with Hep G2 CDX tumors, and the tissue samples shown in FIGS. 12A-12C and 13A-13C were from mice with AsPC-1 CDX tumors. For these tissue samples, the mice were injected with libraries of human CAR T cells, where the CAR design constructs were integrated into the T cell genome and contained phage promoters upstream of a variable barcode. At different time points after the CAR T cells had infiltrated into the tumors, tumor tissue samples were collected and prepared by standard FFPE preparation techniques. Briefly, mice were euthanized, the tumor was dissected out, fixed with formaldehyde or formalin, and embedded in paraffin. The paraffin-embedded blocks were cut into 5 μm thick sections and mounted onto glass slides.

RNA Transcription from Phage Promoter in Fixed Cells Or Tissue

Tissue samples prepared by FFPE above were deparaffinized, rehydrated, and underwent antigen retrieval. Tissue samples were optionally bleached to reduce endogenous background autofluorescence signal using hydrogen peroxide and light. For in vitro cell samples, cells were washed in PBS, fixed, and permeabilized. For both tissue samples and in vitro cell samples, T7 (or other phage polymerase) reaction mix (buffer, rNTPs, phage polymerase, and RNase inhibitor) was added to the sample and the reaction was incubated with the sample at a constant uniform temperature (37-42° C.) for the specified reaction time (3 h-18 h) to generate RNA from DNA using the phage promoter. After reaction incubation, the sample was washed and newly transcribed RNA were fixed in place (e.g., using formaldehyde).

The tissue or cell samples then underwent reverse transcription to generate cDNA from RNA transcripts by hybridization of a primer and treatment with a reverse transcriptase enzyme. The cDNA generated was then fixed in place (e.g., using formaldehyde or glutaraldehyde) and all or a portion of the complementary RNA was optionally digested. cDNA was then used as a target for DNA padlock binding to the sequences on the 5′ and 3′ sides of the barcode, gap-fill, and ligation to generate a circular DNA template containing the nucleic acid sequence of interest. A primer was hybridized to the circular DNA and rolling circle amplification was performed on the circular template to generate many repeated ssDNA copies of the nucleic acid sequence of interest. Tissue or cell samples were washed and ssDNA amplicons were fixed. Sequencing-by-synthesis (SBS) was performed to readout the barcode sequence downstream of the T7 promoter by first adding a sequencing primer and then adding fluorescent nucleotides with reversible terminator sequences, to enable incorporation of only a single nucleotide per round. Fluorophores and reversible terminators were cleaved from the sample following imaging of each round, and the SBS process was repeated to get the full barcode sequence of interest. Tissue or cell samples were stained with DAPI to mark cell nuclei.

For each round of SBS, fluorescent nucleotide signal was imaged and read out in situ by fluorescent microscopy, with one fluorescent channel per nucleotide and one fluorescent channel to detect DAPI signal. Fields-of-view for FFPE tissue section images were selected to cover either all or a portion of the tissue section, and fields-of-view for in vitro images were selected to cover either all or a portion of the well from 96-well plate. The fields-of-view were sometimes partially overlapping to enable downstream computational image stitching.

Raw microscopy images first underwent illumination correction. Illumination correction was performed separately for each channel and each round of imaging, because shading artifacts can change between channels and rounds. Overlapping tiles were aligned for stitching using nuclear images and the phase cross correlation approach from the scikit-image Python package. The resulting whole slide images of different rounds were aligned to a reference round using scikit-image's SIFT algorithm for keypoint detection and RANSAC for estimating the transformation. Nucleus and cell segmentation were performed using nuclear and actin-stained images, respectively. The segmentation was performed by a convolutional neural network approach that was specifically trained on tissue images followed by a watershed algorithm. The barcode detection consists of several steps. First. Laplacian of Gaussian filters of different sizes were applied to each image to remove background noise. Next, images were tiled and resized before spot detection. Spots were filtered based on the filtered and raw mass to remove dim or small spots caused by artifacts. Subsequently, spots were mapped between channels and those that were present at similar intensity in more than one channel were removed. The remaining spots were mapped to cells and finally full barcodes were reconstructed using the cell identification and a fuzzy join for mapping locations between rounds allowing for a shift of up to 3 pixels in x and y directions. Barcodes of the present invention can be designed to have a minimal Hamming distance of 3, which can allow for one mismatch or one missing round in barcode identification.

In another experiment, samples were prepared from multiple tissue types. Tissue was prepared and treated with the T7 reaction, reverse transcription, cDNA amplification, and SBS as described above. FIGS. 9A-9C depict tissue samples from mice with orthotopic gastric PDX tumor, FIGS. 10A-10C and 11A-11C depict tissue samples from mice with Hep G2 CDX tumors, and FIGS. 12A-12C and 13A-13C depict tissue samples from mice with AsPC-1 CDX tumors. For FIGS. 9A-9C, the mouse was injected with a CAR T cell library of 7 unique CAR designs, each with a unique barcode. For FIGS. 10A-10C, the mouse was injected with a CAR T cell library of 9 unique CAR designs, each with a unique barcode.

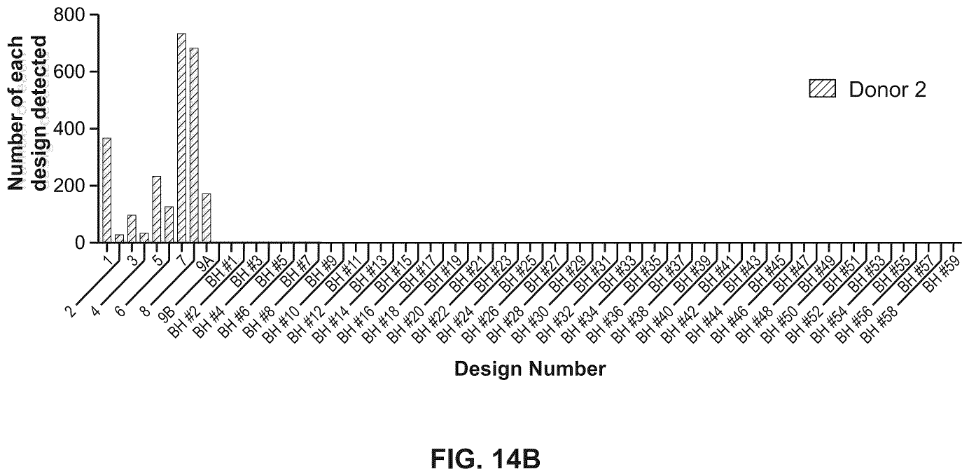

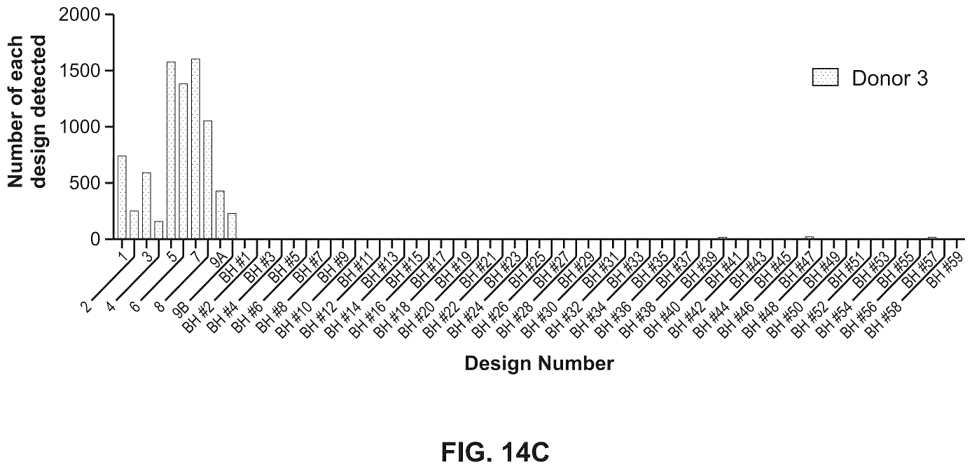

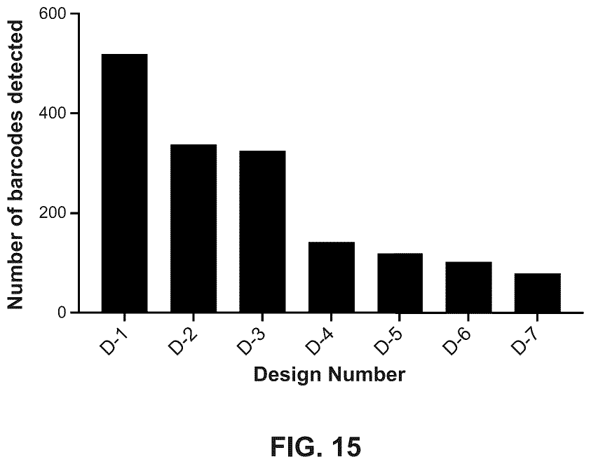

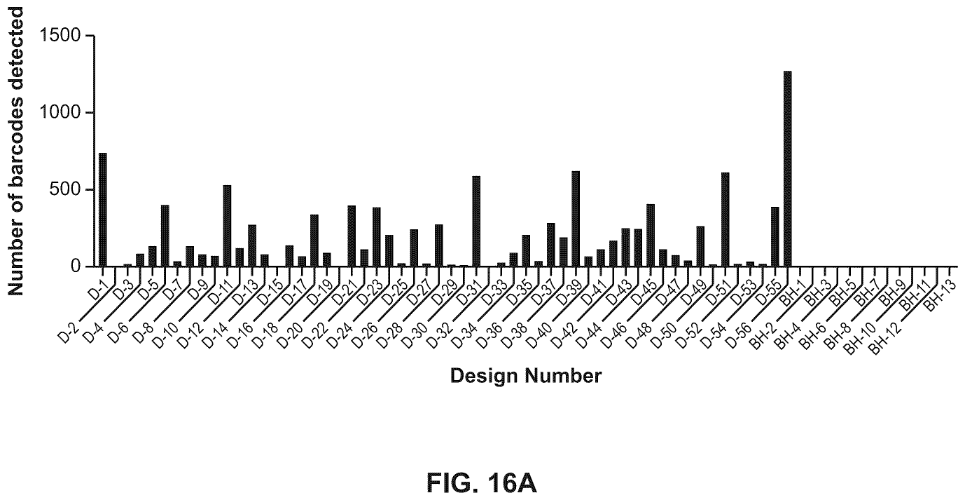

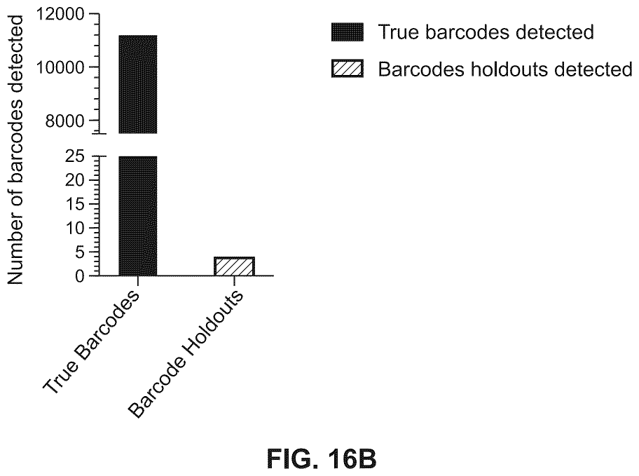

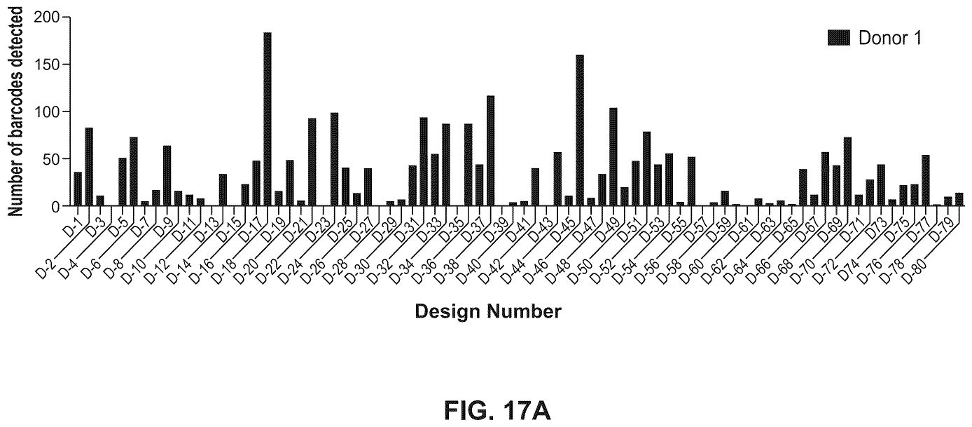

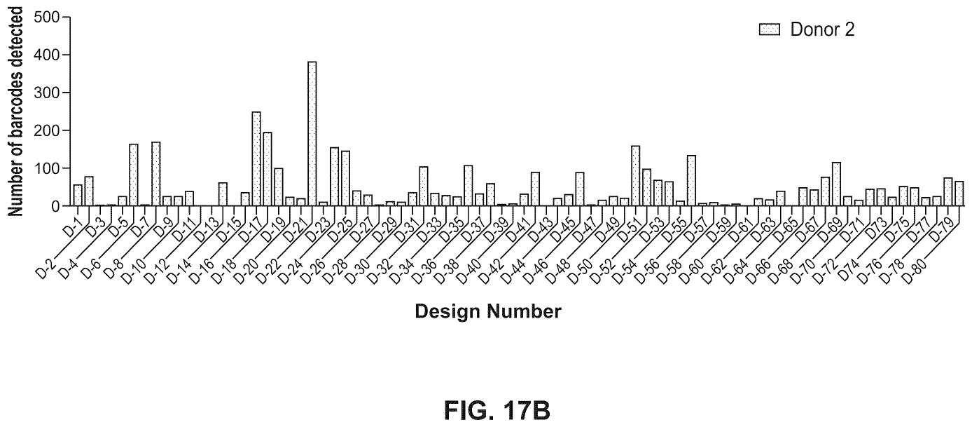

These results indicate that T7 amplification followed by in situ sequencing system can be used to detect the presence and localization of sequences of interest from multiple in vivo sample types. Quantifications of barcodes detected from these tissue samples using the method detailed in above are shown in FIGS. 14A-14C and Table 1 (both corresponding to the sample in FIGS. 10A-10C), FIG. 15 and Table 2 (both corresponding to the sample in FIGS. 9A-9C), FIGS. 16A and 16B and Table 3 (both corresponding to the sample in FIGS. 11A-11C), FIGS. 17A and 17B and Table 4 (both corresponding to the sample in FIGS. 12A-12C), and Table 5 (corresponding to the sample in FIGS. 24A-24F). The quantifications in these figures and tables show that the presence and number of barcodes contained in the tissue samples were highly detected using the T7 amplification and SBS readout method described above.

Table 1 depicts barcode detection specificity for expected barcodes present within a tissue sample. A library of 9 chimeric antigen receptor (CAR) designs, each with a unique barcode following the T7 promoter, were introduced into mice with pre-implanted HepG2 tumors, and then these tumors were extracted to generate FFPE tissue sections. Sixty negative control barcodes, indicated as Barcode Holdout (BH) designs #1-59 and also including either Design 9A or 9B, were not contained in any constructs in the library. As shown in the table, unique CAR design numbers 1-8 were part of the 9mer library for all 3 donors, whereas design number 9A and 9B had different barcodes but the same CAR design, with design 9A contained only in the 9mer library Donors 1 and 2, and design 9B only in the 9mer library for Donor 3. In summary, the majority of barcode holdouts were not highly detected in the FFPE tissue sections, whereas barcodes that were part of the 9 CAR library were detected at higher frequencies in T cells from all 3 donors.

TABLE 1

| | | | | Number | Number | Number |
|---|---|---|---|---|---|---|
| | | | | of each | of each | of each |
| | | Barcode | | barcode | barcode | barcode |
| | | Included | | detected | detected | detected |
| | | in | | from | from | from |
| | Nucleotides | Donor 1 | Included | Donor 1 | Donor 2 | Donor 3 |
| Design | Read out | and | in | tissue | tissue | tissue |
| Number | using SBS | Donor 2? | Donor 3? | section | section | section |
| 1 | TCAGA | yes | yes | 1191 | 364 | 744 |
| 2 | CCATA | yes | yes | 45 | 22 | 261 |
| 3 | TAGTC | yes | yes | 228 | 93 | 599 |
| 4 | TACAG | yes | yes | 93 | 26 | 159 |
| 5 | GTTCT | yes | yes | 1763 | 230 | 1588 |
| 6 | ACATG | yes | yes | 272 | 123 | 1388 |
| 7 | TTCGG | yes | yes | 2977 | 737 | 1609 |
| 8 | ACTTC | yes | yes | 3669 | 681 | 1058 |
| 9A | TAAGG | yes | no | 238 | 168 | 2 |
| 9B | TCGGA | no | yes | 9 | 3 | 238 |
| BH #1 | CTGTA | no | no | 0 | 0 | 0 |
| BH #2 | GCGAA | no | no | 1 | 0 | 0 |
| BH #3 | ACTGT | no | no | 0 | 1 | 0 |
| BH #4 | GGTCA | no | no | 0 | 2 | 1 |
| BH #5 | CAATG | no | no | 0 | 1 | 13 |
| BH #6 | GTAAT | no | no | 0 | 0 | 0 |
| BH #7 | CCGAA | no | no | 2 | 0 | 0 |
| BH #8 | AGTAG | no | no | 1 | 0 | 0 |
| BH #9 | ATAGG | no | no | 4 | 1 | 1 |
| BH #10 | GACTT | no | no | 0 | 0 | 0 |
| BH #11 | AGGCA | no | no | 0 | 1 | 0 |
| BH #12 | CGAAG | no | no | 1 | 0 | 0 |
| BH #13 | AGACC | no | no | 2 | 0 | 0 |
| BH #14 | CGCAT | no | no | 0 | 0 | 0 |
| BH #15 | TATGA | no | no | 1 | 0 | 0 |
| BH #16 | CAGGT | no | no | 0 | 0 | 0 |
| BH #17 | TGACG | no | no | 2 | 0 | 1 |
| BH #18 | AGTGA | no | no | 1 | 0 | 0 |
| BH #19 | ATGCG | no | no | 0 | 0 | 1 |
| BH #20 | TCGAG | no | no | 2 | 0 | 0 |
| BH #21 | AGCAA | no | no | 0 | 0 | 1 |
| BH #22 | TGAAC | no | no | 0 | 0 | 0 |
| BH #23 | TTGCT | no | no | 2 | 0 | 2 |
| BH #24 | TGCAC | no | no | 0 | 0 | 2 |
| BH #25 | GAGCG | no | no | 6 | 4 | 4 |
| BH #26 | GTACG | no | no | 0 | 1 | 0 |
| BH #27 | CATTA | no | no | 0 | 0 | 0 |
| BH #28 | ACTAA | no | no | 2 | 0 | 0 |
| BH #29 | GCCTG | no | no | 3 | 0 | 6 |
| BH #30 | CAAGA | no | no | 1 | 0 | 2 |
| BH #31 | TACCG | no | no | 0 | 0 | 2 |
| BH #32 | CTAAC | no | no | 0 | 0 | 0 |
| BH #33 | TTGAG | no | no | 0 | 0 | 0 |
| BH #34 | GAATG | no | no | 0 | 0 | 8 |
| BH #35 | AACGC | no | no | 2 | 0 | 1 |
| BH #36 | TGCTG | no | no | 1 | 3 | 11 |
| BH #37 | CTGAC | no | no | 0 | 0 | 0 |
| BH #38 | TTACG | no | no | 2 | 0 | 0 |
| BH #39 | ACGCT | no | no | 1 | 0 | 0 |
| BH #40 | ATCGG | no | no | 32 | 4 | 26 |
| BH #41 | ACGTT | no | no | 1 | 1 | 0 |
| BH #42 | GTTAC | no | no | 0 | 0 | 0 |
| BH #43 | TGGCG | no | no | 9 | 6 | 5 |
| BH #44 | CTCAT | no | no | 0 | 0 | 0 |
| BH #45 | ACTCG | no | no | 6 | 0 | 3 |
| BH #46 | AGCGA | no | no | 2 | 0 | 0 |
| BH #47 | TCGTC | no | no | 14 | 4 | 29 |
| BH #48 | CTGCA | no | no | 0 | 0 | 0 |
| BH #49 | GCACT | no | no | 10 | 0 | 4 |
| BH #50 | CATGT | no | no | 0 | 0 | 0 |
| BH #51 | AAGCT | no | no | 0 | 0 | 1 |
| BH #52 | GCTAT | no | no | 0 | 0 | 0 |
| BH #53 | CGATG | no | no | 0 | 0 | 5 |
| BH #54 | GGATA | no | no | 3 | 2 | 2 |
| BH #55 | CGGTA | no | no | 0 | 0 | 6 |
| BH #56 | ATGTC | no | no | 3 | 2 | 4 |
| BH #57 | GTAGG | nc | no | 29 | 6 | 10 |
| BH #58 | AGTCT | no | no | 0 | 0 | 4 |
| BH #59 | GATAC | no | no | 0 | 0 | 0 |

Table 2 depicts barcode detection of a library of 7 CAR designs, each with a unique barcode following the T7 promoter. This pool of 7 CAR designs was injected into a mouse with a patient-derived xenograft tumor that was orthotopically implanted in the mouse stomach, and then the tumor was extracted and processed into FFPE tissue sections. All 7 barcoded designs were well detected.

TABLE 2

| | | | Number of each |
|---|---|---|---|
| | Design | Nucleotides | barcode |
| Design | Number | Read out | detected in |
| Number | on Graph | using SBS | tissue section |
| Design #1 | D-1 | CTGTAAG | 519 |
| Design #2 | D-2 | GTAGGTG | 337 |
| Design #3 | D-3 | ACCGCTA | 325 |
| Design #4 | D-4 | TTCGGTA | 142 |
| Design #5 | D-5 | TCGCGCT | 119 |
| Design #6 | D-6 | AGGACTG | 102 |
| Design #7 | D-7 | CAATGAC | 79 |

Table 3 depicts specificity and scalability for barcode detection in vivo from tissue samples. A library of 56 CAR designs, each with a unique barcode following the T7 promoter, are denotated as designs numbers 1-56. Thirteen Barcode Holdouts are negative control barcoded constructs that were not included in the library. T cells were transduced with barcoded CAR library and injected into a HepG2 cell line-derived xenograft tumor mouse model to generate an FFPE tumor section. Barcodes comprising the 56 CAR design library were highly detected, while barcode holdouts were lowly or not detected.

TABLE 3

| | | | | Number of |
|---|---|---|---|---|
| | | | Barcode | each barcode |
| | Design | Nucleotides | Included | detected |
| | name on | Read out | in | from tissue |
| Design Number | Graph | using SBS | Pool? | section |
| Design #1 | D-1 | TCAGACA | yes | 736 |
| Design #2 | D-2 | CTGTAAG | yes | 6 |
| Design #3 | D-3 | GCGAATC | yes | 17 |
| Design #4 | D-4 | ACTGTCA | yes | 85 |
| Design #5 | D-5 | CCATACT | yes | 135 |
| Design #6 | D-6 | TAGTCTC | yes | 401 |
| Design #7 | D-7 | GGTCATC | yes | 35 |
| Design #8 | D-8 | TACAGCA | yes | 134 |
| Design #9 | D-9 | GTAATGG | yes | 82 |
| Design #10 | D-10 | CCGAAGT | yes | 70 |
| Design #11 | D-11 | AGTAGCG | yes | 533 |
| Design #12 | D-12 | ATAGGCA | yes | 121 |
| Design #13 | D-13 | GACTTCA | yes | 272 |
| Design #14 | D-14 | AGGCATA | yes | 81 |
| Design #15 | D-15 | CGAAGCT | yes | 4 |
| Design #16 | D-16 | AGACCTG | yes | 140 |
| Design #17 | D-17 | CGCATAA | yes | 67 |
| Design #18 | D-18 | GTTCTGC | yes | 337 |
| Design #19 | D-19 | TATGACG | yes | 91 |
| Design #20 | D-20 | CAGGTAG | yes | 4 |
| Design #21 | D-21 | ACATGCC | yes | 398 |
| Design #22 | D-22 | TGACGTT | yes | 114 |
| Design #23 | D-23 | TTCGGTA | yes | 388 |
| Design #24 | D-24 | ATGCGCT | yes | 206 |
| Design #25 | D-25 | TCGAGTG | yes | 22 |
| Design #26 | D-26 | AGCAAGA | yes | 244 |
| Design #27 | D-27 | TGAACGG | yes | 21 |
| Design #28 | D-28 | TTGCTAG | yes | 276 |
| Design #29 | D-29 | TGCACTA | yes | 14 |
| Design #30 | D-30 | GAGCGAT | yes | 10 |
| Design #31 | D-31 | GTACGTA | yes | 589 |
| Design #32 | D-32 | CATTACC | yes | 5 |
| Design #33 | D-33 | AGTCTGG | yes | 26 |
| Design #34 | D-34 | ACTAAGC | yes | 89 |
| Design #35 | D-35 | GCCTGTA | yes | 207 |
| Design #36 | D-36 | CAAGAGA | yes | 36 |
| Design #37 | D-37 | TACCGTC | yes | 284 |
| Design #38 | D-38 | CTAACGA | yes | 190 |
| Design #39 | D-39 | TTGAGAC | yes | 620 |
| Design #40 | D-40 | GAATGGA | yes | 68 |
| Design #41 | D-41 | AACGCGT | yes | 113 |
| Design #42 | D-42 | TGCTGGA | yes | 169 |
| Design #43 | D-43 | TCGGAAG | yes | 251 |
| Design #44 | D-44 | CTGACTC | yes | 245 |
| Design #45 | D-45 | TTACGGC | yes | 407 |
| Design #46 | D-46 | ACGCTGT | yes | 109 |
| Design #47 | D-47 | ATCGGAC | yes | 75 |
| Design #48 | D-48 | ACGTTAG | yes | 42 |
| Design #49 | D-49 | GTTACTG | yes | 266 |
| Design #50 | D-50 | TGGCGAA | yes | 16 |
| Design #51 | D-51 | CTCATGC | yes | 615 |
| Design #52 | D-52 | AGCGAAG | yes | 18 |
| Design #53 | D-53 | TCGTCAA | yes | 32 |
| Design #54 | D-54 | CTGCAGA | yes | 18 |
| Design #55 | D-55 | ACTTCGG | yes | 396 |
| Design #56 | D-56 | GCACTAG | yes | 1267 |
| Barcode Holdout #1 | BH-1 | TAAGGCT | no | 0 |
| Barcode Holdout #2 | BH-2 | CATGTCT | no | 0 |
| Barcode Holdout #3 | BH-3 | CAATGAC | no | 1 |
| Barcode Holdout #4 | BH-4 | AAGCTCC | no | 0 |
| Barcode Holdout #5 | BH-5 | GCTATCG | no | 2 |
| Barcode Holdout #6 | BH-6 | AGTGACC | no | 0 |
| Barcode Holdout #7 | BH-7 | CGATGTA | no | 0 |
| Barcode Holdout #8 | BH-8 | GGATACG | no | 0 |
| Barcode Holdout #9 | BH-9 | CGGTATT | no | 1 |
| Barcode Holdout #10 | BH-10 | ATGTCCG | no | 0 |
| Barcode Holdout #11 | BH-11 | GTAGGAT | no | 0 |
| Barcode Holdout #12 | BH-12 | GATACGC | no | 0 |
| Barcode Holdout #13 | BH-13 | ACTCGTC | no | 0 |

Table 4 depicts barcode detection of a library of 80 CAR designs, each with a unique barcode following the T7 promoter, from FFPE tissue sections from AsPC-1 cell line-derived xenografts. Designs include structural changes to the CAR (CAR #1-17), logic-gated CARs (Logic Gate #1-2), and armored CARs (Armor #1-61). Designs from each of these classes (CAR structural changes, logic-gated CARs, and armored CARs) were detected. Non-detected barcodes may indicate constructs with CAR designs that negatively impact T cell function or fitness within the tumor.

TABLE 4

| | | | | Number of | Number of |
|---|---|---|---|---|---|
| | | | | each | each |
| | | | | barcode | barcode |
| | | | | detected | detected |
| | Design | Nucleotides | Barcode | from Donor | from Donor |
| Design | Name on | read out | Included | 1 Tissue | 2 Tissue |
| Number | Graph | using SBS | in Pool? | Section | Section |
| CAR-1 | D-1 | CTGCAGA | yes | 36 | 57 |
| CAR-2 | D-2 | TCAGACA | yes | 83 | 79 |
| CAR-3 | D-3 | AATCGGA | yes | 11 | 3 |
| CAR-4 | D-4 | GCATTGC | yes | 1 | 4 |
| CAR-5 | D-5 | CATGTCT | yes | 51 | 27 |
| CAR-6 | D-6 | TAGTCTC | yes | 73 | 165 |
| CAR-7 | D-7 | TACAGCA | yes | 5 | 4 |
| CAR-8 | D-8 | CTGTAAG | yes | 17 | 171 |
| CAR-9 | D-9 | CAATGAC | yes | 64 | 27 |
| CAR-10 | D-10 | GTAATGG | yes | 16 | 27 |
| CAR-11 | D-11 | CCGAAGT | yes | 12 | 40 |
| CAR-12 | D-12 | AGTAGCG | yes | 8 | 1 |
| CAR-13 | D-13 | ATAGGCA | yes | 0 | 0 |
| CAR-14 | D-14 | GACTTCA | yes | 34 | 63 |
| CAR-15 | D-15 | AGGCATA | yes | 0 | 0 |
| CAR-16 | D-16 | ATCACGG | yes | 23 | 37 |
| CAR-17 | D-17 | ACGCTGT | yes | 48 | 251 |
| Logic | D-18 | CGAAGCT | yes | 184 | 196 |
| Gate-1 | | | | | |
| Logic | D-19 | ACCGCTA | yes | 16 | 102 |
| Gate-2 | | | | | |
| Armor-1 | D-20 | AGACCTG | yes | 49 | 25 |
| Armor-2 | D-21 | GTTCTGC | yes | 6 | 21 |
| Armor-3 | D-22 | TTGCTAG | yes | 93 | 383 |
| Armor-4 | D-23 | GAGCGAT | yes | 1 | 11 |
| Armor-5 | D-24 | ATGTCCG | yes | 99 | 156 |
| Armor-6 | D-25 | CGCATAA | yes | 41 | 147 |
| Armor-7 | D-26 | AAGCTCC | yes | 14 | 42 |
| Armor-8 | D-27 | TATGACG | yes | 40 | 31 |
| Armor-9 | D-28 | GCTATCG | yes | 1 | 4 |
| Armor-10 | D-29 | CAGGTAG | yes | 5 | 13 |
| Armor-11 | D-30 | AAGTGCA | yes | 7 | 12 |
| Armor-12 | D-31 | ACATGCC | yes | 43 | 37 |
| Armor-13 | D-32 | TGACGTT | yes | 94 | 105 |
| Armor-14 | D-33 | AGTGACC | yes | 55 | 35 |
| Armor-15 | D-34 | CGATGTA | yes | 87 | 29 |
| Armor-16 | D-35 | GGATACG | yes | 0 | 26 |
| Armor-17 | D-36 | TTCGGTA | yes | 87 | 109 |
| Armor-18 | D-37 | TGAACGG | yes | 44 | 34 |
| Armor-19 | D-38 | GTACGTA | yes | 117 | 61 |
| Armor-20 | D-39 | AGTCTGG | yes | 1 | 6 |
| Armor-21 | D-40 | GATACGC | yes | 4 | 7 |
| Armor-22 | D-41 | ACGTTAG | yes | 5 | 33 |
| Armor-23 | D-42 | TGCACTA | yes | 40 | 91 |
| Armor-24 | D-43 | CGGTATT | yes | 1 | 2 |
| Armor-25 | D-44 | AGCAAGA | yes | 57 | 22 |
| Armor-26 | D-45 | GCCTGTA | yes | 11 | 32 |
| Armor-27 | D-46 | TAAGGCT | yes | 160 | 90 |
| Armor-28 | D-47 | CATTACC | yes | 9 | 4 |
| Armor-29 | D-48 | CCATACT | yes | 34 | 17 |
| Armor-30 | D-49 | ACTGTCA | yes | 104 | 27 |
| Armor-31 | D-50 | GGTCATC | yes | 20 | 22 |
| Armor-32 | D-51 | GTTACTG | yes | 48 | 161 |
| Armor-33 | D-52 | TGGCGAA | yes | 79 | 99 |
| Armor-34 | D-53 | CTCATGC | yes | 44 | 70 |
| Armor-35 | D-54 | ACTCGTC | yes | 56 | 66 |
| Armor-36 | D-55 | AGCGAAG | yes | 4 | 15 |
| Armor-37 | D-56 | TCGTCAA | yes | 52 | 135 |
| Armor-38 | D-57 | ATGAGGA | yes | 1 | 8 |
| Armor-39 | D-58 | ATTCGAG | yes | 4 | 10 |
| Armor-40 | D-59 | ATGCGCT | yes | 16 | 4 |
| Armor-41 | D-60 | TACCGTC | yes | 2 | 5 |
| Armor-42 | D-61 | CTAACGA | yes | 0 | 1 |
| Armor-43 | D-62 | TTGAGAC | yes | 8 | 21 |
| Armor-44 | D-63 | TCGAGTG | yes | 3 | 18 |
| Armor-45 | D-64 | ACTAAGC | yes | 6 | 41 |
| Armor-46 | D-65 | CAAGAGA | yes | 2 | 3 |
| Armor-47 | D-66 | GAATGGA | yes | 39 | 50 |
| Armor-48 | D-67 | AACGCGT | yes | 12 | 44 |
| Armor-49 | D-68 | TGCTGGA | yes | 57 | 78 |
| Armor-50 | D-69 | TCGGAAG | yes | 43 | 117 |
| Armor-51 | D-70 | CTGACTC | yes | 73 | 27 |
| Armor-52 | D-71 | TTACGGC | yes | 12 | 17 |
| Armor-53 | D-72 | ACTTCGG | yes | 28 | 46 |
| Armor-54 | D-73 | GCACTAG | yes | 44 | 47 |
| Armor-55 | D-74 | TAGAGGT | yes | 7 | 25 |
| Armor-56 | D-75 | AACTAGC | yes | 22 | 53 |
| Armor-57 | D-76 | TAACGAG | yes | 23 | 50 |
| Armor-58 | D-77 | CACTGCT | yes | 54 | 24 |
| Armor-59 | D-78 | ATCCTGA | yes | 2 | 27 |
| Armor-60 | D-79 | GCTTAGA | yes | 10 | 76 |
| Armor-61 | D-80 | CGTAGGA | yes | 14 | 67 |

Table 5 depicts barcode detection of a library of 7 uniquely barcoded CAR designs from FFPE tissue sections from an orthotopic gastric patient-derived xenograft mouse tumor. The CAR library contained 6 constructs with the barcode after the T7 promoter and one construct with the barcode after both the T7 and SP6 promoters. From left-to-right, table columns list the design number, the design barcodes read out using the phage polymerase system followed by SBS, whether the specific design contained the barcode after the T7 promoter, whether the specific design contained the barcode after the SP6 promoter, the number of each barcoded design detected in an FFPE tissue section treated with T7 for barcode amplification, and the number of each barcoded design detected in an FFPE tissue section treated with T7 for barcode amplification. The results in this table show that both T7 and SP6 phage polymerases efficiently and specifically amplify nucleic acid barcode signal from the constructs containing the relevant promoter. This demonstrates that the phage promoter barcode amplification system used here is not restricted to a single phage polymerase enzyme, meaning multiple polymerases can be used to amplify sequences from multiple unique constructs within the same or across different samples of interest.

TABLE 5

| | | | | Number | |
|---|---|---|---|---|---|
| | | | | of each | Number |
| | | | | barcode | of each |
| | | | | detected | barcode |
| | | | | in T7 | detected |
| | | | | treated | in SP6 |
| | | | | tissue | tissue |
| Design | Barcode | T7 | SP6 | section | section |
| Design #1 | ACCGCTA | x | | 890 | 1 |
| Design #2 | AGGACTG | x | | 343 | 2 |
| Design #3 | CAATGAC | x | | 355 | 0 |
| Design #4 | CTGTAAG | x | | 1755 | 12 |
| Design #5 | GTAGGTG | x | x | 694 | 728 |
| Design #6 | TCGCGCT | x | | 625 | 0 |
| Design #7 | TTCGGTA | x | | 950 | 1 |

In another experiment, barcodes were detected from libraries of multiple DNA construct types, where constructs encoded different protein-coding or non-protein coding designs and each construct also contained a variable nucleic acid barcode downstream of the T7 promoter. Tissue samples were prepared and subject to the T7 reaction, reverse transcription, cDNA amplification, and sequencing-by-synthesis method for barcode detection as described above. FIGS. 10A-10C depict tissue samples containing CAR T cells made from a DNA construct library containing 9 chimeric antigen receptors (CARs), including armored and unarmored CARs. The 9 uniquely barcoded CAR designs in the library from the cells depicted in FIGS. 10A-10C are listed in Table 1 and quantified in FIGS. 14A-14C. FIGS. 9A-9C depicts tissue samples containing CAR T cells made from a DNA construct library containing 7 CARs. The 7 uniquely barcoded CAR designs in the library from the cells depicted in FIGS. 9A-9C are listed in Table 2 and quantified in FIG. 15. FIGS. 11A-11C depicts tissue samples containing CAR T cells made from a DNA construct library containing 56 CARs, including armored and unarmored CARs. The 56 uniquely barcoded CAR designs in the library from the cells depicted in FIGS. 11A-11C are listed in Table 3 and quantified in FIGS. 16A and 16B. FIGS. 12A-12C depicts tissue samples containing CAR T cells made from a DNA construct library containing 80 CAR designs, including structurally distinct unarmored CARs, armored CARs, and logic-gated CARs. The 80 uniquely barcoded CAR designs in the library from the cells depicted in FIGS. 12A-12C are listed in Table 4 and quantified in FIGS. 17A and 17B. FIGS. 13A-13C depicts tissue samples containing CAR T cells made from a DNA construct library containing 12 CARs, including one unarmored CAR and 11 CARs each co-expressed with a different shRNA. These results indicate that T7 barcode amplification followed by in situ SBS can be used to determine the presence and number of multiple types of protein-coding and non-protein coding DNA construct libraries from tissue samples. These results also show that the T7 barcode amplification system can be used to detect and quantify target sequences from DNA construct libraries independent of the number of constructs comprising the library.

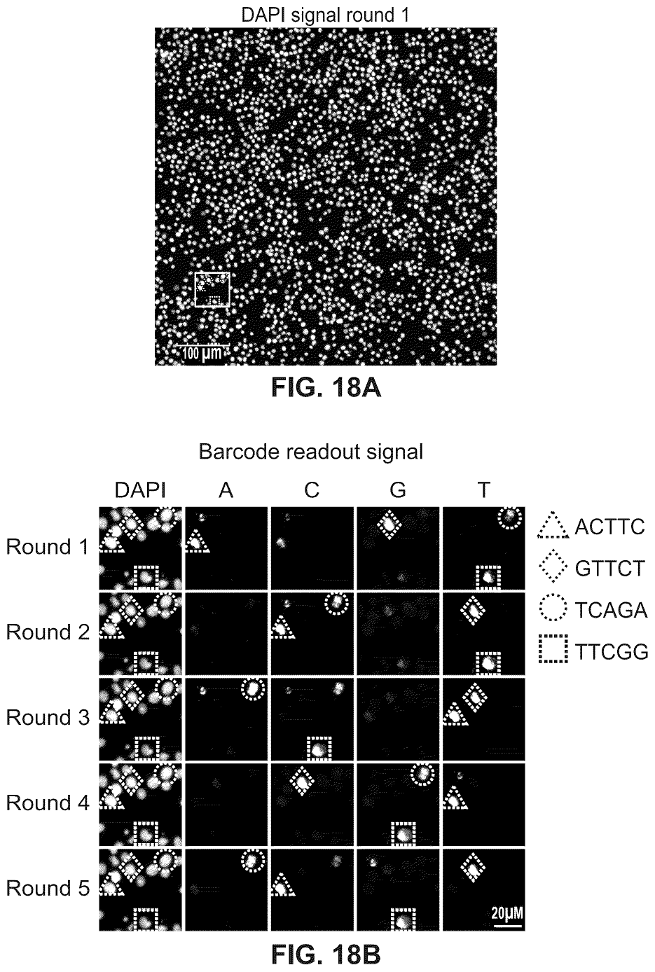

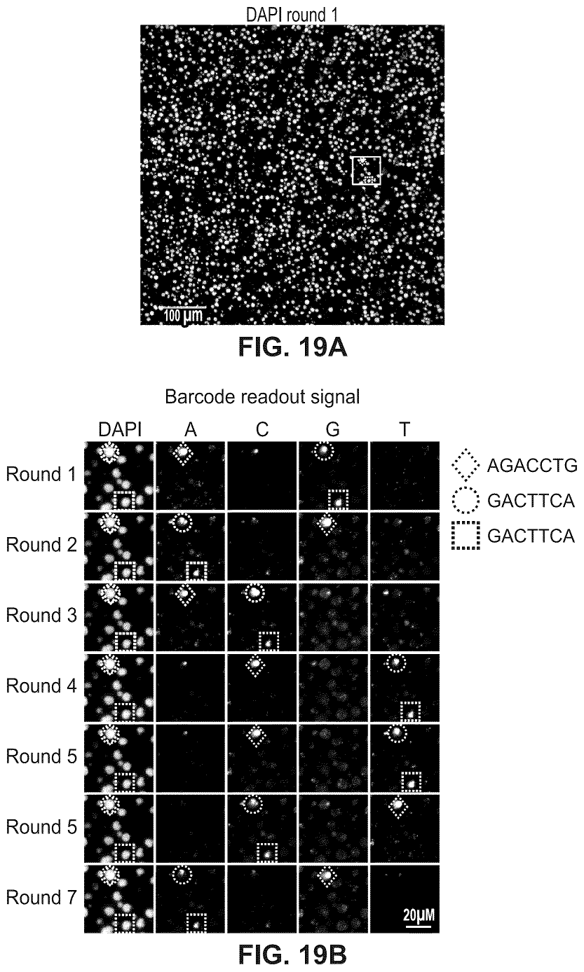

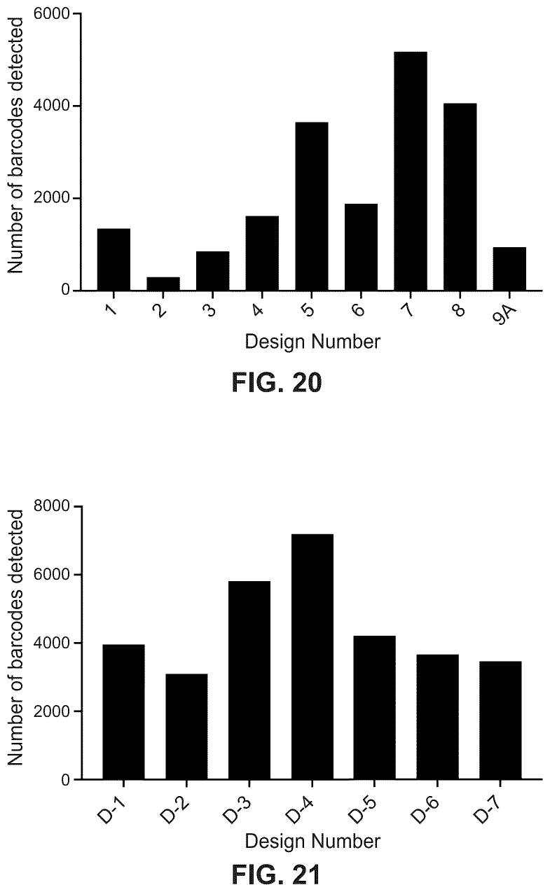

In another experiment, cells were used in vitro following genetic integration of the phage promoter and barcode containing construct described above, without mouse injection or tissue preparation. Cells were washed in PBS, fixed, and permeabilized. The T7 reaction, reverse transcription, cDNA amplification, and SBS were performed as described above. FIGS. 18A, 18B, 19A and 19B depict in vitro T cells with CAR design constructs containing the T7 promoter upstream of a variable barcode. FIG. 20 depicts quantification and Table 1 lists the designs of the 9 uniquely barcoded CARs from the in vitro T cell library depicted in FIGS. 18A and 18B. FIG. 21 depicts quantification from in vitro barcode detection of the 7 uniquely barcoded CARs described in Table 2, demonstrating that all barcoded designs were highly detected in vitro. These results indicated that T7 amplification followed by in situ SBS can be used to detect and quantify barcodes or nucleic acid sequences of interest from both in vitro cells and in vivo tissue samples. The provided methods are advantageous for screening of drug candidates as they allow the simultaneous screening of multiple drug candidates in a single sample. The provided methods also provide enhanced detection of nucleic acid sequences in situ.

### Example 7: Detection of the Variable Barcode Sequences is Accurate

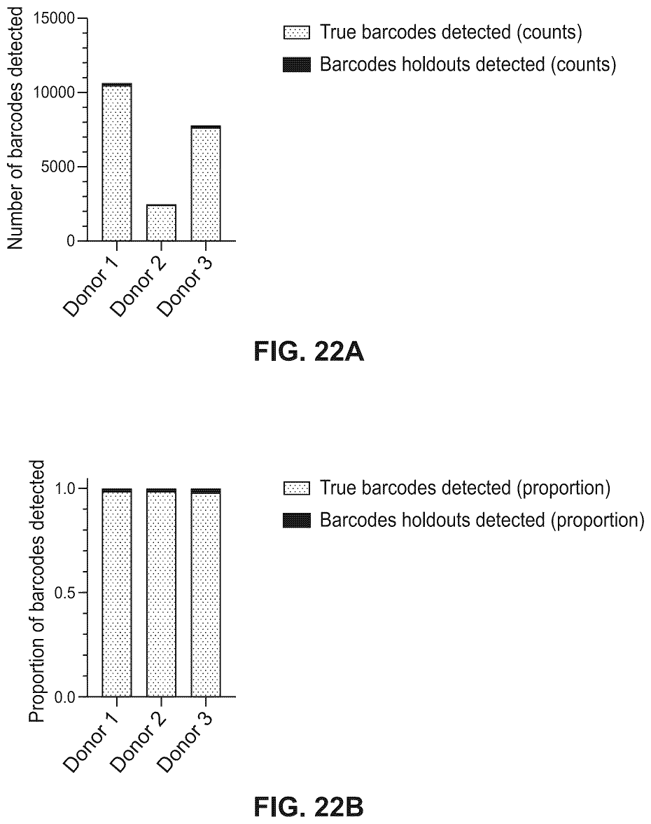

Barcode detection accuracy was determined by comparing detection of barcoded DNA constructs known to be contained within tissue samples to detection of barcoded DNA construct not contained in the tissue samples, termed “barcode holdouts.” In this example, a library of 9 CAR constructs was designed, each with a unique barcode downstream of the T7 promoter. Sixty negative control CAR constructs were also designed to contain a barcode and the T7 promoter, but these constructs were excluded from the library. The nucleotide sequence of the barcode holdouts and the barcodes on the 9 CAR library were designed using the same design requirements, such as avoiding homopolymeric sequences. The 9 construct CAR library was transduced into T cells isolated from one of three different human lymphocyte donors using the approaches described in Example 6 above. Construct-containing cells were then introduced into mice with pre-implanted Hep G2 CDX tumors, as described in Example 6. Tissue sections were prepared and subject to T7 amplification, followed by reverse transcription, cDNA amplification, and in situ SBS as described in Example 6. The number of barcoded constructs present in the samples containing cells derived from the three donors was determined. FIGS. 14A-14C and 22A-22B depict quantifications of barcodes detected in vivo from tissue samples containing the T cells transduced with the 9 barcoded CAR library. Table 1 lists the CAR designs, nucleotide barcodes readout using the T7 system, whether each barcoded CAR construct was introduced into T cells derived from each of the three donors, and the number of detected barcodes from these samples. Number of barcodes detected was compared between the 9 CAR designs introduced to the sample (true barcodes) and 60 negative control designs, termed barcode holdouts (BH), which were not contained in the construct library introduced to the samples. Barcodes from the 9 introduced CAR designs were detected at high numbers in the samples, while barcode holdouts were lowly or not detected. This accuracy of detection by the T7 amplification system for barcoded constructs contained in the tissue relative to barcode holdouts was observed in samples with cells made from all three donors.

To further establish accuracy, barcoded CAR design 9A and barcoded CAR design 9B were designed to be the same CAR but with different barcodes. Design 9A was included only in the library introduced into cells from donors 1 and 2, and design 9B was included only in the library introduced into cells from donor 3. Barcode detection demonstrated that CAR designs 1-8, common to cells from all 3 donors, was highly present for all 3 donor cell tissue samples, while design 9A was highly detected only in cells from donors 1 and 2 and design 9B was highly detected only in cells from donor 3. Notably, CAR designs in the 9 CAR library would be expected to impact the number of cells with each design in the tissue independently of the barcodes, so detection of equal numbers of barcodes from each design was not expected. Overall, these results show that T7 promoter-based amplification and detection by cDNA amplification and SBS accurately distinguishes DNA constructs in in vivo samples based on the unique identifying nucleotide sequences of the barcodes contained in each construct.

In another experiment, accuracy of barcode detection was also demonstrated from tissue samples containing T cells transduced with a 56 barcoded CAR construct library, which were generated, processed, and underwent barcode amplification as described above. FIGS. 16A and 16B depict quantifications comparing the number of barcodes detected between 56 CAR designs contained in the sample and 13 barcode holdouts. Table 3 lists the CAR design number, design name notation from FIGS. 16A and 16B, nucleotide barcodes read out using the T7 system, whether each barcoded CAR construct was contained in the 56 CAR library pool introduced into the cell sample, and the number of detected barcodes from tissue samples containing these cells. Barcode detection from tissue samples with the 56 CAR T cell library demonstrates that barcode holdouts were not detected or were weakly detected, while barcoded constructs that were part of the library were detected at high frequencies. As described for the 9 CAR library above, the different CAR designs comprising the 56 CAR library would be expected to impact the number of cells with each design in the tissue, independent of the barcode, so detection of equal numbers of barcodes from each design was not expected. Overall, these results indicate that the phage promoter system for in vivo nucleic acid amplification and detection by cDNA amplification and SBS is highly accurate for design constructs and barcodes included in a library. This accuracy can allow for enhanced detection and quantification of nucleic acid species in situ, as well as simultaneous testing of multiple constructs with unique barcodes in a sample.

### Example 8: Amplification of Variable Barcode Sequencing Using Either T7 or SP6 Promoters within a Biological Sample, Followed by Amplification of eDNA Product

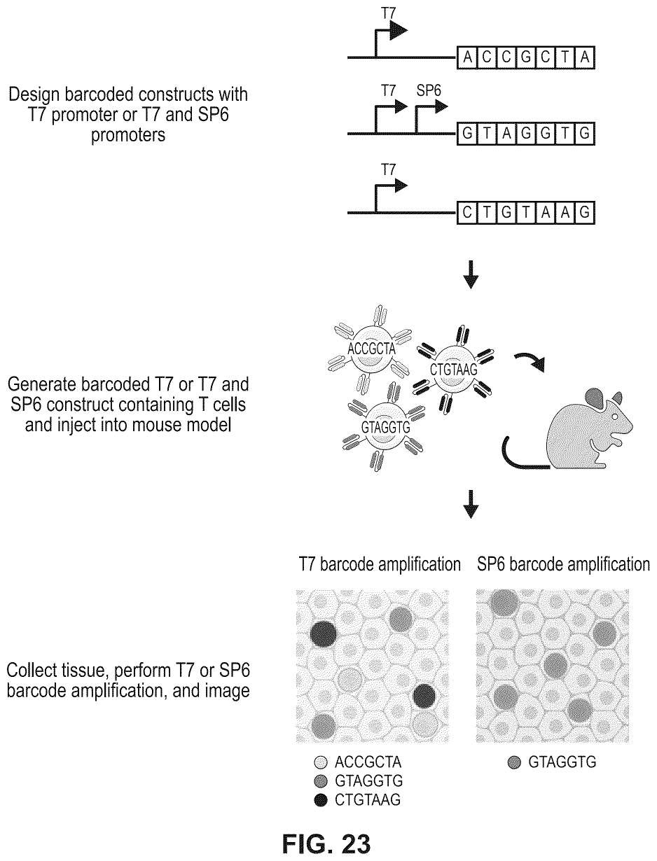

Tissue samples were prepared as in Example 6 and subject to in vivo barcode amplification using either an T7 or SP6 RNA polymerase. To generate tissue samples. T cells were transduced with a library of seven uniquely barcoded CAR designs. The CAR library contained six constructs with the T7 RNA polymerase promoter upstream of the nucleic acid barcode and one construct with both the T7 RNA polymerase promoter and the SP6 RNA polymerase promoter upstream of the nucleic acid barcode (T7+SP6 promoter construct). T cells containing the T7 promoter constructs or the T7+SP6 promoter construct were injected into an orthotopic gastric PDX tumor mouse model and tissue samples were prepared as described in Example 6 above. Barcode amplification was performed on the tissue samples using either the T7 RNA polymerase reaction or the SP6 RNA polymerase reaction, and subsequent reverse transcription, cDNA amplification, and in situ SBS. FIG. 23 shows a schematic depicting the experiment comparing barcode detection using either the T7 or SP6 promoters. The library of uniquely barcoded CARs was designed to contain multiple constructs with the barcode after the T7 promoter and one construct with the barcode after both the T7 and SP6 promoters. The barcoded construct library was transduced into CAR T cells and injected into a mouse model. The mouse was sacrificed, tissue was harvested, and barcodes were amplified with either T7 or SP6 phage polymerase and subsequent reverse transcription, cDNA amplification, and in situ SBS. Tissue imaging post SBS identified barcode signal from all library constructs in the T7 treated sample, but only the SP6 promoter containing construct in the SP6 treated sample. FIGS. 24A-24F depict tissue samples containing CAR T cells made from the seven CAR library with barcodes detected using either the T7 or SP6 promoter system. The T7 amplification system detected barcodes from all seven T7 promoter containing CAR designs, while the SP6 amplification system highly detected only the barcode from the CAR design containing the SP6 promoter. This example demonstrates the specificity the phage promoter amplification system for detecting the nucleic acid sequence of interest only from the constructs that contain the phage promoter recognized by the RNA polymerase enzyme, consistent with the sequence specificity of these enzymes. Table 5 shows quantification of the number of barcodes detected from the tissue samples in FIGS. 24A-24F treated with either the T7 or SP6 barcode amplification method. The number of barcodes from the construct containing the T7 and SP6 promoter was similar between the tissue samples treated with the T7 and SP6 polymerases, with 694 and 728 barcodes detected, respectively. This result shows that both the T7 and SP6 polymerases can efficiently amplify nucleic acid barcode signal in constructs containing the relevant promoter. This result also demonstrated that the phage promoter and SBS detection system was generalizable and not specific to a single RNA polymerase enzyme or promoter and that other enzymes and promoters can be utilized in the methods provided for herein.

### Example 9: Amplification of Multiple Nucleic Acid Sequences from Multiple Phage Promoters within a Single Cell

The phage promoter barcode amplification system was used to distinguish multiple nucleic acid sequences of interest from a single cell. For this example, 16 CAR design constructs were generated, each with a unique barcode following the T7 promoter. T cells were then transduced by lentivirus at a multiplicity of infection to introduce 0, 1, 2, or more constructs per individual cell. The cells were fixed in vitro and subject to T7 promoter driven transcription, reverse transcription, cDNA amplification, and in situ SBS as described in Example 6. Barcode detection was performed to determine the number and identity of CAR DNA constructs contained in each cell. FIGS. 19A and 19B depict microscopy images of T cells transduced with the CAR library. The cell indicated by the diamond and circle shapes contains two uniquely barcoded CAR constructs while the cell indicated by the square shape contains a single uniquely barcoded CAR construct. These results indicate that multiple nucleic acid sequences of interest, each downstream of a phage promoter, can be detected and distinguished from within a single cell using the phage amplification system.

### Example 10: Combination of Protein Detection with Amplification of Variable Barcode Sequence Using an Integrated Promoter within an In Vivo Tissue Sample, with Amplification of cDNA Product

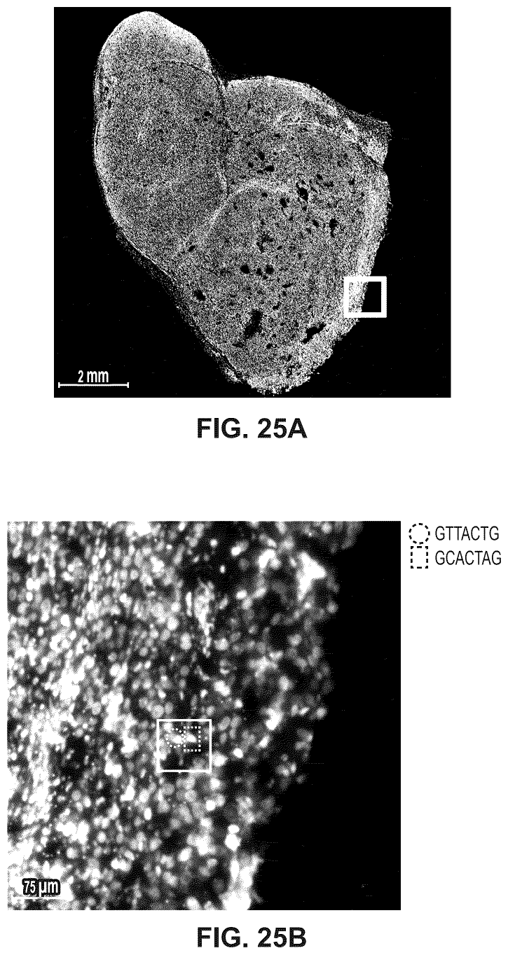

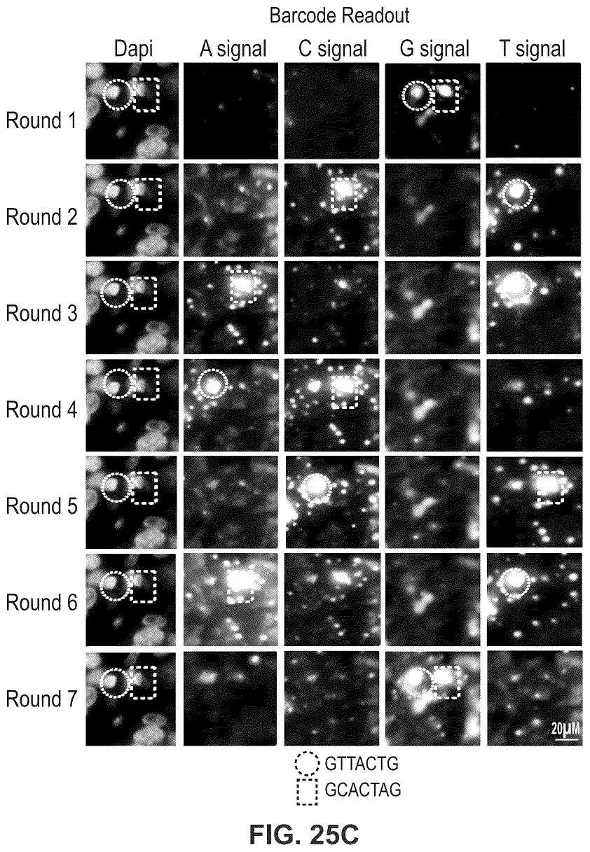

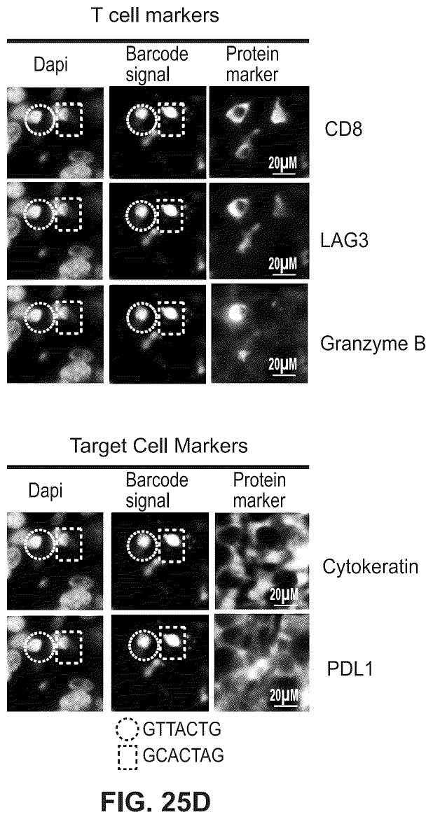

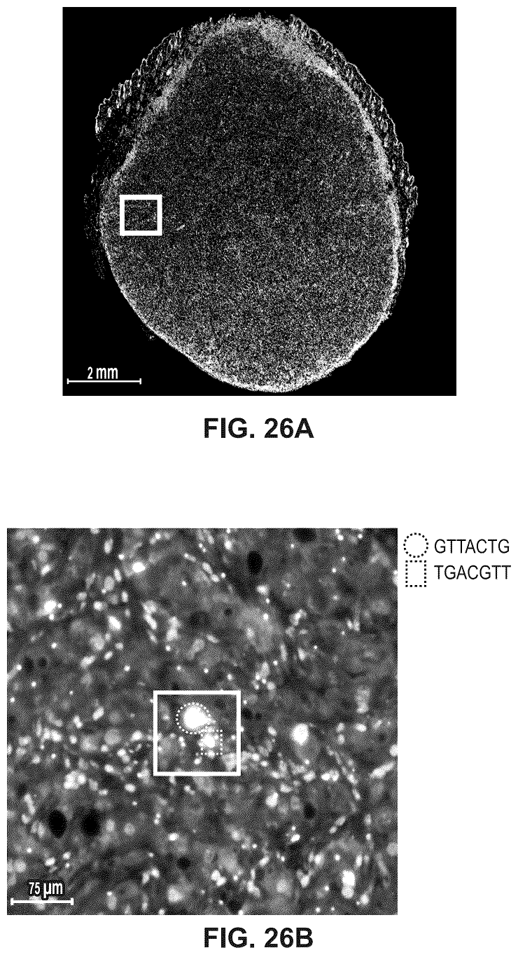

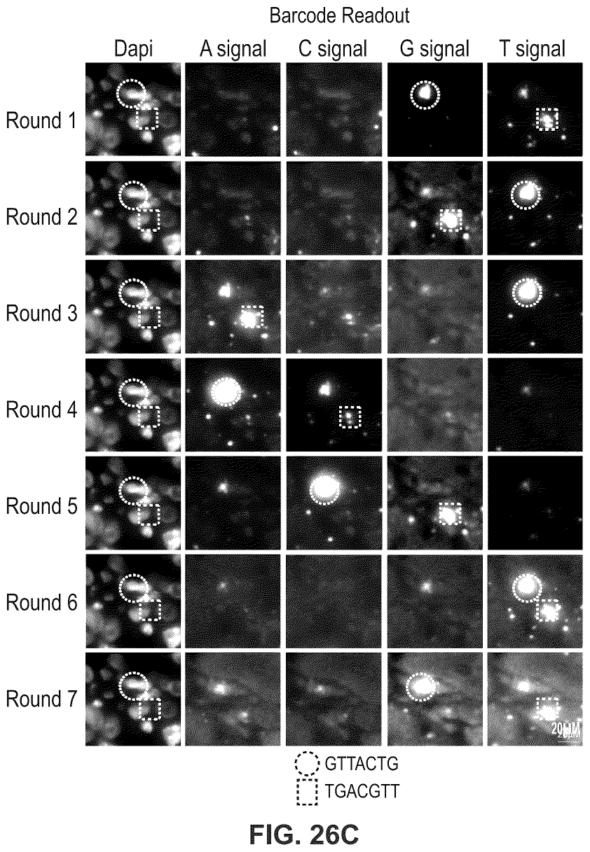

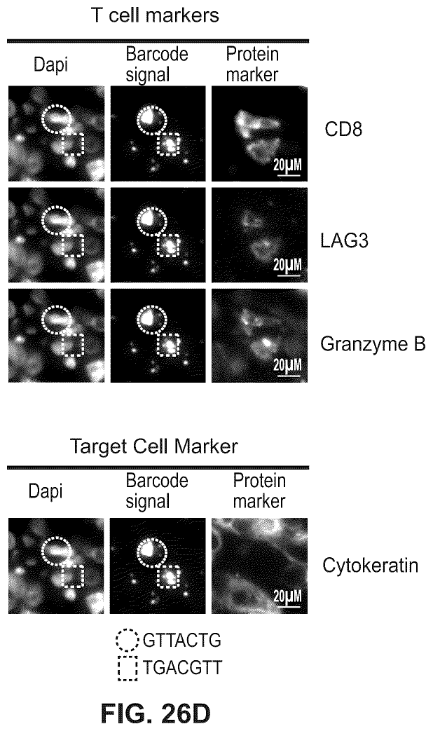

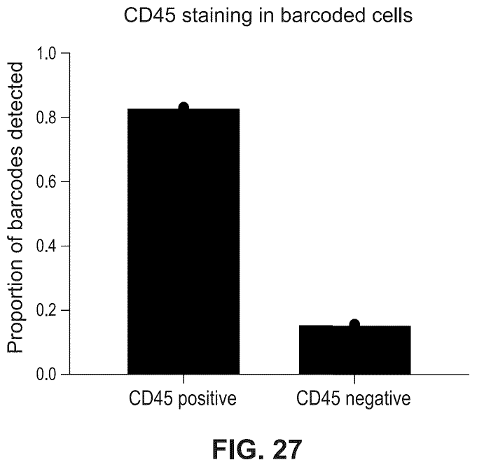

Construct design, cell line engineering, mouse models, and FFPE tissue section processing were performed as described in Example 6 above. To combine protein detection with nucleic acid sequence amplification using the T7 promoter, tissue sections were deparaffinized, rehydrated, and underwent antigen retrieval. Tissue was incubated in blocking buffer to reduce non-specific antibody binding and tissue was washed with PBS. Tissue sections were incubated with primary or fluorophore conjugated antibodies overnight. After primary antibody incubation, tissue sections were washed and optionally incubated with fluorophore conjugated secondary antibodies. DAPI staining was performed to mark cell nuclei and tissue sections were imaged to detect protein signal from antibody-stained tissue. In some examples, tissue was stained iteratively to detect additional proteins using antibodies through fluorophore removal with stringent washes and additional rounds of antibody staining performed as above. Tissue sections were then washed with formamide/SSC, washed with PBS, and underwent barcode amplification using the approach described in Example 6 including T7 promoter transcription, reverse transcription, cDNA amplification, in situ SBS and imaging for barcode detection. For some samples, tissue staining with antibodies was performed to detect proteins after the barcode detection process was complete. FIGS. 25A-25D depict microscopy images from tissue sections from a HepG2 CDX tumor mouse model containing T cells transduced with a library of 56 uniquely barcoded CAR constructs. The tissue section underwent barcode amplification with T7 RNA polymerase and subsequent reverse transcription, cDNA amplification, and in situ SBS. Protein analytes CD8, Granzyme B, LAG3, and PDL1 were stained prior to T7 amplification and barcode detection by SBS, whereas cytokeratin was stained after T7 amplification and barcode detection by SBS. Prior to sacrificing the mouse and harvesting the tumor, the mouse was injected with a CAR T cell library of 56 unique designs, including both armored and unarmored CAR constructs, each with a unique barcode. FIGS. 26A-26D depict microscopy images from tissue sections from an AsPC-1 CDX tumor mouse model containing T cells transduced with a library of 80 uniquely barcoded CAR constructs. The tissue section underwent barcode amplification with T7 RNA polymerase and subsequent reverse transcription, cDNA amplification, and in situ SBS. Protein analytes CD8, Granzyme B, LAG3 were stained prior to T7 amplification and barcode detection by SBS, whereas cytokeratin was stained after T7 amplification and barcode detection by SBS. Prior to sacrificing the mouse and harvesting the tumor, the mouse was injected with a CAR T cell library of 80 unique designs, including both armored and unarmored CAR constructs, each with a unique barcode. Tissue sections depicted in FIGS. 25A-25D and 26A-26D underwent T7-driven barcode amplification and antibody staining for protein markers. Antibody signal from staining of T cell specific proteins, including CD8, LAG3, and Granzyme B was specifically localized to T cells and was not strongly detected in cancer cells. Antibody signal from staining of tumor cancer cell specific proteins, including cytokeratin and PD-L1, was specifically localized to target cancer cells and was not strongly observed in T cells. Signal from both in situ barcode amplification and T cell specific protein antibody staining was detected in CAR containing T cells. This indicates that the T7 amplification system can be used to identify and quantify barcoded construct containing cells in combination with physiologically relevant protein detection. Signal from in situ barcode amplification in these tissue samples was more strongly detected in T cells than cancer cells, further demonstrating that the nucleic acid barcodes amplified by the T7 system were specifically found in the cells into which the constructs were introduced. FIG. 27 depicts quantification of the proportion of cells with T7 amplified barcoded constructs detected that were defined as positive or negative for CD45 antibody staining in an FFPE tissue sample. This sample contained cells transduced with the 80 CAR library described in Table 4. Tissue underwent barcode detection via T7 promoter-based amplification as described above and protein staining with an antibody for CD45, which is endogenously expressed by T cells. CD45 positive cells indicate T cells, while CD45 negative cells indicate other cell types that are not T cells including AsPC-1 cancer cells. Barcodes were highly detected in CD45 positive cells relative to CD45 negative cells, indicating that the T7 system for nucleic acid amplification specifically identified barcodes in cells containing the barcoded CAR constructs.

### Example 11: Combination of RNA Detection with Amplification of Variable Barcode Sequence Using an Integrated Promoter within an In Vivo Tissue Sample, with Amplification of cDNA Product

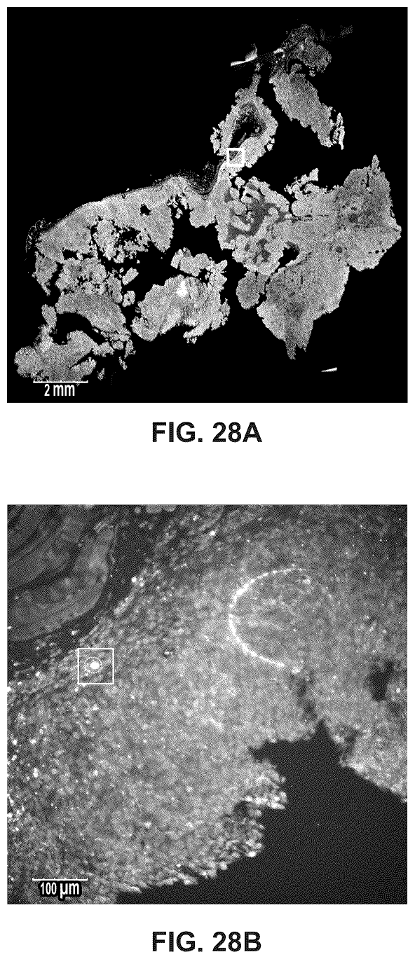

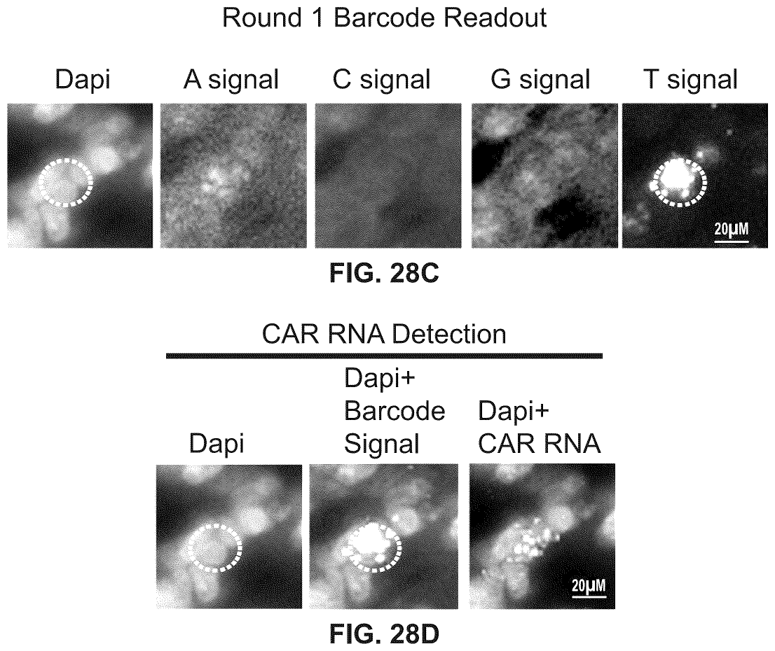

Construct design, cell line engineering, mouse models, and FFPE tissue section processing were performed as described in Example 6 above. To combine RNA detection with nucleic acid sequence amplification using the T7 promoter, tissue sections were deparaffinized, rehydrated, and underwent antigen retrieval and were washed in PBS. Tissue sections were then subject to hybridization chain reaction (HCR) by first pre-hybridizing samples through incubation with hybridization buffer containing formamide and SSC. Single stranded DNA HCR probes with sequences specific for binding to the RNA transcript target sequence (EGFR and WPRE) were added to the tissue sample in hybridization buffer and incubated overnight to allow the probes to anneal to the target RNA. Tissue was washed in SSC to remove non-specifically bound HCR probes and HCR DNA hairpin amplifiers tethered to fluorophores were added to the sample in amplification buffer. Tissue sections were incubated with the amplifiers overnight and washes were performed with SSC and PBS to remove excess HCR hairpins. Tissue was stained with DAPI and imaged for fluorescent HCR signal specific to the RNA target. To perform T7 barcode amplification and detection, the HCR signal was removed from tissue through RNase treatment, and the sample then underwent T7 promoter transcription, reverse transcription, cDNA amplification by rolling circle amplification, in situ SBS using a sequencing primer, imaging, and barcode detection as described in Example 6. FIGS. 28A-28D depicts microscopy images from tissue sections from a Hep G2 CDX tumor mouse model containing T cells transduced with a library of 56 uniquely barcoded CAR constructs. The tissue depicted underwent HCR to amplify RNA from the barcoded CAR construct, followed by T7 driven barcode amplification. In the sample depicted, signal from both in situ barcode amplification and HCR amplification of CAR RNA was detected in the CAR containing T cell. This demonstrates the specificity of the T7 system for detecting barcodes in cells that express the barcoded construct RNA.

In summary, the examples and methods provided for herein demonstrate the ability to detect and analyze one or more nucleic acid sequences of interest based on the presence, abundance, and localization of the constructs due to the combination of steps provided for herein. These methods allow for the spatial detection of nucleic acids in situ within a cell or tissue sample. These methods surprisingly allow for this with high efficiency and accuracy, which is a significant advantage when evaluating where and how much of a construct is present. This is especially important given that techniques for in situ sequencing are inefficient with low detection efficiencies (See, Moffitt J R, et al. Nat Rev Genet. 2022 December: 23 (12): 741-759), especially in FFPE samples where other genomics technologies have reported detection efficiencies 5-10 times lower than in fresh frozen samples (See, Moses L, et al. Nat Methods. 2022 May: 19 (5): 534-546). Further, cell types and tissues vary substantially in RNA content (See, Walker D G, et al. Cell Tissue Bank. 2016 September; 17 (3): 361-375), and the amount of RNA content impacts detection rates of genes by genomics technologies (See, Mereu E, et al. Nat Biotechnol. 2020 June; 38 (6): 747-755), highlighting the need for new approaches to perform nucleic acid detection with higher efficiencies. These methods therefore allow for the screening of a greater number of constructs (each with a unique barcode) with fewer rounds of sequencing, improving both time- and cost-efficiency. These advantages can lead to, for example, faster screening or lead selection or lead optimization of drug candidates including gene therapies or cell therapies or biologics. These advantages can also lead to, for example, a better understanding of the role of different genes in a biological system. These are just some of the advantages and technical effects of the methods and compositions provided for herein.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of determining the presence and/or localization of a nucleic acid sequence of interest operably linked to an exogenous promoter in one or more fixed mammalian cells within a biological sample in situ, wherein the biological sample is fixed using a solution comprising formaldehyde or paraformaldehyde, the method comprising:
(a) reacting, within the one or more fixed mammalian cells, (i) a DNA molecule comprising the nucleic acid sequence of interest operably linked to the exogenous promoter, and (ii) an RNA polymerase that specifically recognizes the exogenous promoter, thereby to generate an RNA transcript of the nucleic acid sequence of interest;
(b) reacting the RNA transcript in situ with a reverse transcriptase enzyme to generate a cDNA molecule comprising the nucleic acid sequence of interest; and
(c) in situ sequencing the cDNA molecule to visualize the nucleic acid sequence of interest in the one or more fixed mammalian cells thereby to determine the presence and/or localization of the nucleic acid sequence of interest.

2. The method of claim 1, wherein the method comprises, prior to step (a), contacting the one or more fixed mammalian cells with an RNase to degrade endogenous RNA molecules.

3. The method of claim 1, wherein the DNA molecule is, or is derived from, an exogenous nucleic acid molecule that is introduced to the one or more mammalian cells prior to fixation.

4. The method of claim 3, wherein the nucleic acid sequence of interest is a barcode polynucleotide.

5. The method of claim 3, wherein the exogenous nucleic acid molecule is incorporated into a genome of the one or more mammalian cells by viral transduction, site-specific nucleases, or site-specific recombinases.

6. The method of claim 5, wherein the exogenous DNA-nucleic acid molecule is introduced to the one or more mammalian cells using a viral vector selected from a lentiviral vector, a retroviral vector, an adenovirus vector, an HSV vector, a baculovirus vector, a virus-like particle, a pseudotyped virus-like capsid, an oncolytic viral vector, or an AAV vector.

7. (canceled)

8. The method of claim 1, wherein the DNA molecule is generated by reverse transcribing with a DNA primer that hybridizes to a target RNA comprising the nucleic acid sequence of interest in the one or more fixed mammalian cells, wherein the DNA primer comprises:
(a) a 5′ nucleic acid sequence comprising the exogenous promoter, and
(b) a 3′ nucleic acid sequence that is complementary to a portion of the target RNA flanking the nucleic acid sequence of interest.

9. The method of claim 8, wherein the method further comprises converting the DNA molecule to double-stranded DNA by second-strand synthesis.

10. The method of claim 8, wherein the 5′ nucleic acid sequence of the DNA primer comprising the exogenous promoter is dsDNA.

11. The method of claim 8, wherein the target RNA molecule is wholly or partially digested following synthesis of the DNA molecule.

12. The method of claim 1, wherein the in situ sequencing is sequencing-by-synthesis, sequencing-by-ligation, or sequencing-by-avidity.

13. The method of claim 1, wherein:
(a) the exogenous promoter is a phage promoter, or a transcriptionally active variant thereof, and the RNA polymerase is a phage RNA polymerase;
(b) the exogenous promoter is a bacterial promoter and the RNA polymerase is a bacterial RNA polymerase;
(c) the exogenous promoter is a eukaryotic promoter and the RNA polymerase is a eukaryotic RNA polymerase;
(d) the exogenous promoter is a viral promoter and the RNA polymerase is a viral RNA polymerase; or
(e) the exogenous promoter is a synthetic promoter and the RNA polymerase is a synthetic RNA polymerase.

14. The method of claim 1, wherein the exogenous promoter and the RNA polymerase are selected from the group consisting of:
(i) a T7 promoter, or a transcriptionally active variant thereof, and a T7 RNA polymerase, respectively;
(ii) a T3 promoter, or a transcriptionally active variant thereof, and a T3 RNA polymerase, respectively; and
(iii) an SP6 promoter, or a transcriptionally active variant thereof, and an SP6 RNA polymerase, respectively.

15. The method of claim 1, wherein the DNA molecule further comprises a transcriptional terminator.

16. (canceled)

17. The method of claim 1, wherein the biological sample comprises a formalin-fixed, paraffin-embedded (FFPE) sample comprising the one or more mammalian cells.

18. The method of claim 1, wherein the DNA molecule further comprises one or more polynucleotide sequences encoding exogenous proteins, endogenous proteins, or a mixture of exogenous and endogenous proteins.

19. The method of claim 18, wherein the exogenous proteins are independently selected from the group consisting of a chimeric antigen receptor (CAR), an antibody, a T-cell receptor, a cytokine, a cell-surface receptor, a transcription factor, a signaling protein, and a protease.

20. The method of claim 1, wherein the DNA molecule further comprises:
(a) a polynucleotide sequence encoding an endogenous protein;
(b) a polynucleotide sequence encoding an endogenous RNA;
(c) a polynucleotide sequence encoding an exogenous RNA;
(d) a nucleic acid sequence that alters expression, function, and/or sequence of one or more genes; and/or
(e) a polynucleotide sequence encoding a viral genome.

21. The method of claim 1, wherein the DNA molecule further comprises a polynucleotide sequence encoding a nucleic acid sequence that alters expression, function, and/or sequence of one or more genes.

22. The method of claim 21, wherein the nucleic acid sequence that alters expression, function, and/or sequence of one or more genes is selected from the group consisting of an sgRNA, a gRNA an shRNA, and an miRNA.

23. The method of claim 1, wherein the DNA molecule comprises a second exogenous promoter configured to drive transcription of a second nucleic acid sequence of interest in the presence of a second RNA polymerase that specifically recognizes the second exogenous promoter.

24. The method of claim 23, wherein the second nucleic acid sequence of interest is a second barcode polynucleotide.

25. The method of claim 23, wherein the second exogenous promoter and the second RNA polymerase are selected from the group consisting of:
(i) a T7 promoter, or a transcriptionally active variant thereof, and a T7 RNA polymerase, respectively;
(ii) a T3 promoter, or a transcriptionally active variant thereof, and a T3 RNA polymerase, respectively;
(iii) a SP6 promoter, or a transcriptionally active variant thereof, and a SP6 RNA polymerase, respectively;
(iv) a bacterial promoter or a transcriptionally active variant thereof, and a bacterial RNA polymerase, respectively;
(v) a eukaryotic promoter or a transcriptionally active variant thereof, and a eukaryotic RNA polymerase, respectively;
(vi) a viral promoter or a transcriptionally active variant thereof, and a viral RNA polymerase, respectively; or
(vii) a synthetic promoter and a synthetic RNA polymerase, respectively.

26. The method of claim 1, wherein the DNA molecule further comprises a first padlock-binding sequence and a second padlock-binding sequence, wherein said first and second padlock-binding sequences flank a region comprising the nucleic acid sequence of interest.

27. The method of claim 23, wherein the DNA molecule further comprises a third padlock-binding sequence and a fourth padlock-binding sequence, wherein said third and fourth padlock-binding sequences flank a region comprising the second nucleic acid sequence of interest.

28. The method of claim 26, wherein, prior to in situ sequencing, step (c) further comprises the steps of:
(i) contacting the cDNA with a first padlock probe comprising a 5′ end and a 3′ end, wherein the first padlock probe comprises a 5′ nucleic acid sequence which is reverse complementary to the first padlock-binding site and a 3′ nucleic acid sequence which is reverse complementary to the second padlock-binding site, thereby allowing the 5′ and 3′ nucleic acid sequences to hybridize to the cDNA;
(ii) extending the 3′ end of the first padlock probe through the nucleic acid sequence of interest using a DNA polymerase;
(iii) ligating the 5′ end of the padlock probe to the extended 3′ end of the padlock probe, thereby generating a circular DNA template comprising a nucleic acid sequence reverse complementary to the nucleic acid sequence of interest; and
(iv) using rolling circle amplification of the DNA template to generate additional copies of the nucleic acid sequence of interest.

29. The method of claim 1, wherein the in situ sequencing is performed directly on the cDNA.

30. The method of claim 1, wherein the method does not comprise contacting the one or more fixed mammalian cells with a DNAse.

31. A method of determining the presence and/or localization of a nucleic acid sequence of interest operably linked to an exogenous promoter in one or more fixed mammalian cells within a biological sample in situ, the method comprising:
(a) reacting, within the one or more fixed mammalian cells, (i) a DNA molecule comprising the nucleic acid sequence of interest operably linked to the exogenous promoter, and (ii) an RNA polymerase that specifically recognizes the exogenous promoter, thereby to generate an RNA transcript of the nucleic acid sequence of interest;
(b) reacting the RNA transcript in situ with a reverse transcriptase enzyme to generate a cDNA molecule comprising the nucleic acid sequence of interest; and
(c) in situ sequencing the cDNA molecule to visualize the nucleic acid sequence of interest in the one or more fixed mammalian cells thereby to determine the presence and/or localization of the nucleic acid sequence of interest,
wherein the method does not comprise contacting the one or more fixed mammalian cells with a DNAse.
